(19) United States
(12) Patent Application Publication
(10) Pub. No.: US 2026/0092914 A1
(43) Pub. Date: Apr. 2, 2026

(54) SINGLE ANALYTE CHARACTERIZATION UNDER EQUILIBRIUM BINDING CONDITIONS

(71) Applicant: Nautilus Subsidiary, Inc., Seattle, WA (US)

(72) Inventors: Parag MALLICK, San Mateo, CA (US); Kara JUNEAU, San Carlos, CA (US); Subramanian SANKAR, Danville, CA (US); Aimee SANFORD, San Mateo, CA (US); Robert GROTHE, San Jose, CA (US); Jarrett D. EGERTSON, Rancho Palos Verdes, CA (US); Keith BJORNSON, Fremont, CA (US)

(21) Appl. No.: 19/338,819

(22) Filed: Sep. 24, 2025

Related U.S. Application Data
(60) Provisional application No. 63/700,276, filed on Sep. 27, 2024.

(51) Int. Cl. G01N 33/543; G01N 33/68

(52) U.S. Cl. CPC G01N 33/54306; G01N 33/6803; G01N 2333/9015; G01N 2333/9127

(57) ABSTRACT

Methods and systems are provided for detecting a binding interaction between a binding reagent and an analyte under equilibrium binding conditions. The binding interaction can be detected by the formation of an interaction identification moiety. The interaction identification moiety can be detected even after the binding reagent dissociates from the analyte.

### CROSS-REFERENCE TO RELATED APPLICATIONS

This application claims priority to U.S. Prov. App. 63/700,276 filed Sep. 27, 2024, entitled “SINGLE ANALYTE CHARACTERIZATION UNDER EQUILIBRIUM BINDING CONDITIONS”, which is incorporated by reference in its entirety.

### BACKGROUND

Single analyte characterization can include any method of characterizing a population of analytes such that each individual analyte in the population is characterized. Single analyte characterization can determine the individual analyte characteristics that give rise to the ensemble behavior of a population of analytes. For example, a single analyte characterization assay may determine the activity state of each individual enzyme of a population of enzymes, thereby facilitating understanding of the bulk activity of the population of enzymes.

Some methods of characterizing single analytes depend upon the binding of reagents (e.g., affinity reagents) to individual analytes, and subsequently detecting the binding of the reagents to the individual analytes. It may be preferable to have such binding interactions occur at equilibrium to maximize the quantity of binding interactions that occur. Binding equilibrium between analytes and binding reagents may depend at least in part on the respective concentrations of the analyte and the binding reagents, as well as other factors such as temperature and fluid composition.

### SUMMARY

In an aspect, provided herein is a method of forming a binding profile, comprising: (a) binding affinity reagents to analytes of a plurality of analytes, wherein each of the affinity reagents is individually joined with an affinity reagent identifier moiety, and wherein each of the analytes is individually joined with a unique analyte identifier moiety, (b) for each analyte bound to an affinity reagent, coupling together the affinity reagent identifier moiety and the unique analyte identifier moiety, thereby forming an interaction identification moiety comprising an analyte-specific code from the unique analyte identifier moiety, (c) repeating steps (a) and (b) at least once with differing affinity reagents, (d) detecting for each interaction identification moiety from steps (b) and (c) the analyte-specific code, and (e) for each analyte of the plurality of analytes, forming a binding profile for the analyte, wherein the binding profile comprises a presence or absence of a detected analyte-specific code for the analyte for each of the at least two differing affinity reagents.

In some embodiments, a plurality of analytes can comprise at least about 10⁶ analytes (e.g., at least about 10⁷, 10⁸, 10⁹, 10¹⁰, 10¹¹, 10¹², or more than 10¹² analytes). In some embodiments, a plurality of analytes can comprise two or more species of analytes. In some embodiments, a method may further comprise, based upon binding profiles of analytes of the plurality of analytes, identifying a presence of the two or more species of analytes in the plurality of analytes. In some embodiments, a method may further comprise determining a quantity of each species of the two or more species of analytes in the plurality of analytes. In some embodiments, the two or more species of analytes can comprise at least 100 species of analytes. In some embodiments, the two or more species of analytes can comprise a first species of analyte and a second species of analyte, wherein the first species of analyte and the second species of analyte have a dynamic range of at least about 10⁴ (e.g., a dynamic range of at least about 10⁵, 10⁶, 10⁷, 10⁸, 10⁹, 10¹⁰, or more than 10¹⁰). In some embodiments, a method may further comprise, based upon binding profiles of the analytes of the plurality of analytes, identifying the presence of the first species of analyte and the second species of analyte in the plurality of analytes. In some embodiments, a method may further comprise determining a quantity of the first species of analyte and the second species of analyte in the plurality of analytes.

In some embodiments, a plurality of analytes may comprise a plurality of proteins. In some embodiments, a plurality of proteins may comprise two or more species of proteins, as determined by full-length primary amino acid structure. In some embodiments, a plurality of proteins can comprise two or more species of proteins, as determined by proteoform or isoform.

In some embodiments, forming an interaction identification moiety can comprise ligating a first oligonucleotide comprising the analyte-specific code to a second oligonucleotide comprising the affinity reagent-specific code. In some embodiments, forming an interaction identification moiety can comprise hybridizing a first oligonucleotide comprising the analyte-specific code to a second oligonucleotide comprising the affinity reagent-specific code. In some embodiments, a method may further comprise extending the first oligonucleotide or the second oligonucleotide enzymatically. In some embodiments, an analyte identifier moiety can comprise a polymer strand, wherein the affinity reagent identifier comprises an enzyme. In some embodiments, forming an interaction identification moiety can comprise altering the polymer strand with the enzyme.

In some embodiments, step (c) can comprise: (i) separating the plurality of analytes from a first fluid phase; (ii) removing the first fluid phase, wherein the first fluid phase comprises the affinity reagents; and (iii) contacting a second fluid phase to the plurality of analytes, wherein the second fluid phase comprises a second set of affinity reagents. In some embodiments, a binding specificity of the affinity reagents can differ from a binding specificity of affinity reagents of the second set of affinity reagents.

In some embodiments, a method can further comprise separating the interaction identification moieties from the affinity reagents or from the analytes. In some embodiments, separating the interaction identification moieties from the affinity reagents or from the analytes can occur before step (c). In some embodiments, separating the interaction identification moieties from the affinity reagents or from the analytes can occur after step (c). In some embodiments, a method may further comprise delivering the interaction identification moieties to a detection device, wherein the detection device is configured to identify for each interaction identification moiety the analyte-specific code.

In some embodiments, a method may further comprise characterizing each analyte of the plurality of analytes based upon the binding profile of the analyte. In some embodiments, characterizing each analyte of the plurality of analytes can comprise identifying an analyte of the plurality of analytes. In some embodiments, characterizing each analyte of the plurality of analytes can comprise identifying a proteoform of an analyte of the plurality of analytes. In some embodiments, identifying the proteoform of the analyte of the plurality of analytes can comprise identifying two or more proteoforms of a species of analyte of the plurality of analytes.

In another aspect, provided herein is a composition, comprising: (a) a solid support, wherein the solid support is attached to a plurality of particles, wherein, for each particle of the plurality of particles, the particle is attached to a unique analyte identifier moiety and a single unknown analyte, (b) a plurality of affinity reagents, and (c) a plurality of interaction identification moieties, wherein each interaction identification moiety comprises an analyte-specific code of an analyte identifier moiety.

In some embodiments, an interaction identification moiety of the plurality of interaction identification moieties can further comprise an affinity reagent-specific code. In some embodiments, an interaction identification moiety of the plurality of interaction identification moieties further can comprise an assay sequence-specific code or a vessel-specific code. In some embodiments, an interaction identification moiety of the plurality of interaction identification moieties can be attached to an affinity reagent of the plurality of affinity reagents. In some embodiments, an interaction identification moiety of the plurality of interaction identification moieties can be attached to a particle of the plurality of particles.

In some embodiments, the plurality of affinity reagents can be in a fluid phase and the solid support can be in a solid phase. In some embodiments, the plurality of affinity reagents and the solid support are in a fluid phase.

In some embodiments, an affinity reagent of the plurality of affinity reagents can comprise an affinity reagent identifier moiety. In some embodiments, the affinity reagent can be attached to a single unknown analyte, wherein the affinity reagent identifier moiety can be attached to an analyte identifier moiety co-located with the single unknown analyte. In some embodiments, a composition can further comprise an enzyme molecule attached to the analyte identifier moiety. In some embodiments, the enzyme comprises a ligase or a polymerase. In some embodiments, the enzyme comprises a biotin ligase, a sortase, or a terminal deoxynucleotidyl transferase.

In another aspect, provided herein is a system for characterizing analytes, comprising: (a) a plurality of vessels, wherein each vessel comprises a plurality of particles, wherein the plurality of particles is attached to a plurality of analytes, wherein each analyte of the plurality of analytes is co-localized with an analyte identifier moiety on a particle of the plurality of particles, wherein each analyte identifier moiety comprises a unique analyte-specific code, (b) a library of affinity reagents, wherein the library of affinity reagents comprises two or more pluralities of affinity reagents, wherein each plurality of affinity reagents of the two or more pluralities of affinity reagents differs with respect to binding specificity from any other plurality of affinity reagents of the two or more pluralities of affinity reagents, and wherein each affinity reagent of the library of affinity reagents is attached to an affinity reagent identifier moiety, (c) a detection device, wherein the detection device is configured to receive a plurality of interaction identification moieties, and wherein the detection device is further configured to detect for each interaction identification moiety of the plurality of interaction identification moieties an analyte-specific code, and (d) a fluid transfer device, wherein the fluid transfer device is configured to deliver a plurality of affinity reagents from the library of affinity reagents to a vessel of the plurality of vessels, and wherein the fluid transfer device is further configured to deliver the plurality of interaction identification moieties from a vessel of the plurality of vessels to the detection device.

In some embodiments, a fluid transfer device can be further configured to transfer a plurality of affinity reagents from a first vessel of the plurality of vessels to a second vessel of the plurality of vessels. In some embodiments, a system can further comprise a field-generating device. In some embodiments, the field-generating device can comprise a heating device, a cooling device, a mixing device, a magnetic-field generator or an electric-field generator. In some embodiments, the field-generating device can comprise a light source.

### INCORPORATION BY REFERENCE

All publications, items of information available on the internet, patents, and patent applications cited in this specification are herein incorporated by reference to the same extent as if each individual publication, patent, or patent application was specifically and individually indicated to be incorporated by reference. To the extent publications, items of information available on the internet, patents, or patent applications incorporated by reference contradict the disclosure contained in the specification, the specification is intended to supersede and/or take precedence over any such contradictory material.

### BRIEF DESCRIPTION OF THE DRAWINGS

FIGS. 1A, 1B, 1C, 1D, 1E, and 1F depict steps of a method of detecting binding interactions by the formation of interaction identification moieties, in accordance with some embodiments.

FIGS. 2A, 2B, 2C, 2D, and 2E display various configurations of analyte identifier moieties and binding reagent identifier moieties that can facilitate formation of an interaction identification moiety, in accordance with some embodiments.

FIG. 3 shows a method of enriching a pool of polymer strands for polymer strands that have been formed into interaction identification moieties, in accordance with some embodiments.

FIG. 4 is a graph which illustrates the selection of an optimal binding reagent concentration for a binding reagent that forms superordinate and subordinate binding interactions, in accordance with some embodiments.

FIGS. 5A, 5B, and 5C depict configurations of interaction identification moieties that may facilitate dissociation of the interaction identification moiety from an analyte, binding reagent, or a complex thereof, in accordance with some embodiments.

FIGS. 6A, 6B, and 6C display configurations of an array-based system for detecting interaction identification moieties, in accordance with some embodiments.

FIGS. 7A, 7B, 7C, 7D, and 7E show steps of a method of detecting binding interactions by utilizing a pre-formed pool of interaction identification moieties, in accordance with some embodiments.

FIGS. 8A and 8B illustrate a system for reversibly isolating assay components within a vessel, in accordance with some embodiments.

FIG. 9 depicts a method of forming an interaction identification moiety comprising a plurality of affinity reagent-specific codes, in accordance with some embodiments.

FIGS. 10A, 10B, and 10C provide schematics of methods of obtaining interaction identification moieties, in accordance with some embodiments.

FIGS. 11A, 11B, and 11C show configurations of a one-pot system for analyte characterization, in accordance with some embodiments.

FIGS. 12A and 12B illustrate characterization of analytes under differing binding conditions, in accordance with some embodiments.

FIG. 13 depicts a system of implementing some methods set forth herein, in accordance with some embodiments.

### DETAILED DESCRIPTION

Chemical systems, including biological systems, can include a large amount of complexity due in part to the diversity of chemical and/or biomolecular species present. This complexity can extend up to the scale of a proteome (potentially containing thousands of protein species) or even the scale of a microbiome (containing thousands of protein species from each organismal species in the microbiome). For example, the human proteome may contain in excess of 20,000 unique species of protein, and those protein species may give rise to potentially in excess of 1,000,000 unique proteoforms. Further, in a chemical or biological system with substantial complexity, there can exist a large dynamic range between abundant species of analyte and rare species of analyte. For example, a blood sample from a patient with an incipient form of cancer may provide 10¹⁰ copies of human serum albumin and a single copy of a cancer biomarker.

Bulk assay of complex chemical or biochemical systems may be limited in the amount of information it can provide. Depending upon the mode of detection, a bulk assay of a chemical or biochemical system may lack the sensitivity to detect low-concentration analyte species. This may substantially decrease the dynamic range of such a bulk assay, and restrict the scope of analyte diversity explored by the bulk assay. Single-analyte characterization, in which analytes are interrogated and characterized on a molecule-by-molecule basis, may be preferable for detecting the full dynamic range and species diversity of a complex sample. Single-analyte characterizations may include molecular identification, molecular quantitation, and molecular state determination (e.g., activity state, activation state, degradation state, etc.) Decoding approaches may be useful for characterizing complex mixtures of analytes. Decoding approaches can utilize broad interrogations of populations of analytes to deduce specific identities of members of the population. For example, within a proteome, hundreds or even thousands of species of proteins may contain the amino acid sequence DTR. Likewise, a specific subset of the proteome may contain a tag sequence that marks proteins for transport across the cellular membrane. Accordingly, there may be a substantially smaller number of protein species of the proteome that both contain the epitope DTR and have the membrane-transport tag sequence. These two broad pieces of information combine to provide a smaller list of candidate identities for any analyte that fits this profile. A decoding approach can utilize serial interrogations of a population of single analytes to form an interrogation profile for each analyte of the population of analytes, in which the interrogation profile for the analyte can be utilized to identify or otherwise characterize the analyte. Decoding approaches that may be useful for characterizing an analyte or a population thereof are described in U.S. Pat. No. 11,282,585, and U.S. Patent Publications 2020/0082914A1, 2023/0114905A1, and 2023/0360732A1, each of which is incorporated herein by reference in its entirety.

The present disclosure provides methods and systems for interrogating analytes by binding reagents under equilibrium binding conditions to produce pools of coded moieties that can be detected after each interrogation. Serial formation and detection of pools of coded moieties may be utilized to form binding profiles for each analyte of a population of analytes, and the binding profiles may be utilized for identification and/or characterization of the analytes by a decoding algorithm. The methods and system set forth herein may be especially useful because they do not require optical resolvability between analytes to achieve single-analyte characterization. Accordingly, the methods and systems set forth herein may be amenable to unpatterned arrays or occur within a fluid phase.

### Definitions

Terms used herein will be understood to take on their ordinary meaning in the relevant art unless specified otherwise. Several terms used herein and their meanings are set forth below.

In some of the implementations described herein, the terms “address” and “site” can refer synonymously to a location in an array where a particular analyte (e.g. protein, peptide or unique identifier label) is present. An address can contain a single analyte, or it can contain a population of several analytes of the same species (i.e. an ensemble of the analytes). Alternatively, an address can include a population of different analytes. Addresses are typically discrete. The discrete addresses can be contiguous, or they can be separated by interstitial spaces. An array useful herein can have, for example, addresses that are separated by less than 100 microns, 10 microns, 1 micron, 100 nm, 10 nm or less. Alternatively or additionally, an array can have addresses that are separated by at least 10 nm, 100 nm, 1 micron, 10 microns, or 100 microns. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁷, 1×10⁸, 1×10⁹, 1×10¹⁰, 1×10¹¹, 1×10¹², or more addresses

In some of the implementations described herein, the terms “affinity reagent” and “binding reagent” can refer synonymously to a molecule or other substance that is capable of specifically or reproducibly binding to an analyte (e.g. protein) or moiety (e.g. post-translational modification of a protein). An affinity reagent can be larger than, smaller than or the same size as the analyte. An affinity reagent may form a reversible or irreversible bond with an analyte. An affinity reagent may bind with an analyte in a covalent or non-covalent manner. Affinity reagents may include reactive affinity reagents, catalytic affinity reagents (e.g., kinases, proteases, etc.) or non-reactive affinity reagents (e.g., antibodies or fragments thereof). An affinity reagent can be non-reactive and non-catalytic, thereby not permanently altering the chemical structure of an analyte to which it binds. Affinity reagents that can be particularly useful for binding to proteins include, but are not limited to, antibodies or functional fragments thereof (e.g., Fab′ fragments, F(ab′)₂ fragments, single-chain variable fragments (scFv), di-scFv, tri-scFv, or microantibodies), aptamers, affibodies, affilins, affimers, affitins, alphabodies, anticalins, avimers, miniproteins, DARPins, monobodies, nanoCLAMPs, lectins, or functional fragments thereof. The term “affinity agent” is intended to be synonymous with the term “affinity reagent.”

In some of the implementations described herein, the term “antibody” can refer to a protein that binds to an antigen or epitope via at least one complementarity determining region (CDR). An antibody can include all elements of a full-length antibody. However, an antibody need not be full length and functional fragments can be particularly useful for many uses. The term “antibody” as used herein encompasses full length antibodies and functional fragments thereof.

In some of the implementations described herein, the term “array” can refer to a population of analytes (e.g. proteins) that are associated with unique identifiers such that the analytes can be distinguished from each other. A unique identifier can be, for example, a solid support (e.g. particle or bead), address on a solid support, tag, label (e.g. luminophore), or barcode (e.g. nucleic acid barcode) that is associated with an analyte and that is distinct from other identifiers in the array. Analytes can be associated with unique identifiers by attachment, for example, via covalent bonds or non-covalent bonds (e.g. ionic bond, hydrogen bond, van der Waals forces, electrostatics etc.). An array can include different analytes that are each attached to different unique identifiers. An array can include different unique identifiers that are attached to the same or similar analytes. An array can include separate solid supports or separate addresses that each bear a different analyte, wherein the different analytes can be identified according to the locations of the solid supports or addresses.

In some of the implementations described herein, the term “attached” can refer to the state of two things being joined, fastened, adhered, connected or bound to each other. Attachment can be covalent or non-covalent. For example, a particle can be attached to a protein by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions, adhesion, adsorption, and hydrophobic interactions.

In some of the implementations described herein, the term “binding affinity” or “affinity” can refer synonymously to the strength or extent of binding between an affinity reagent and a binding partner. In some cases, the binding affinity of an affinity reagent for a binding partner may be vanishingly small or effectively zero. A binding affinity of an affinity reagent for a binding partner may be qualified as being a “high affinity,” “medium affinity,” or “low affinity.” A binding affinity of an affinity reagent for a binding partner, affinity target, or target moiety may be quantified as being “high affinity” if the interaction has a dissociation constant of less than about 100 nM, “medium affinity” if the interaction has a dissociation constant between about 100 nM and 1 mM, and “low affinity” if the interaction has a dissociation constant of greater than about 1 mM. Binding affinity can be described in terms known in the art of biochemistry such as equilibrium dissociation constant (KD), equilibrium association constant (KA), association rate constant (kon), dissociation rate constant (koff) and the like. See, for example, Segel, Enzyme Kinetics John Wiley and Sons, New York (1975), which is incorporated herein by reference in its entirety.

In some of the implementations described herein, the term “binding interaction” can refer to a reaction that associates an affinity reagent to an analyte. A binding reaction may be a covalent or non-covalent interaction. A binding interaction may associate an affinity reagent to an analyte for a sufficient length of time to detect a complex formed by the affinity reagent and analyte.

In some of the implementations described herein, the term “binding probability” can refer to the probability that an affinity reagent or probe may be observed to interact with an analyte, for example, within a given binding context. A binding probability may be expressed as a discrete number (e.g., 0.4 or 40%) a matrix of discrete numbers, or as a mathematical model (e.g., a theoretical or empirical model). A binding probability may include one or more factors, including binding specificity, likelihood of locating a target epitope, or the likelihood of binding for a sufficient time to detect a binding interaction.

In some of the implementations described herein, the term “binding profile” can refer to a plurality of binding outcomes for a protein or other analyte. The binding outcomes can be obtained from independent binding observations, for example, independent binding outcomes can be acquired using different affinity reagents, respectively. Alternatively, the binding outcomes can be generated in silico, for example, being derived from a modification of an empirically obtained binding outcome. A binding profile can include empirical measurement outcomes, candidate measurement outcomes, calculated measurement outcomes, theoretical measurement outcomes or a combination thereof. A binding profile can exclude one or more of empirical measurement outcomes, candidate measurement outcomes, calculated measurement outcomes, or theoretical measurement outcomes or putative measurement outcomes. A binding profile can include a vector of binding outcomes.

In some of the implementations described herein, the term “binding specificity” refers to the tendency of a binding reagent to preferentially interact with a given analyte relative to other analytes. A binding reagent may have a calculated, observed, known, or predicted binding specificity for a given analyte. Binding specificity may refer to selectivity for a single analyte in a given sample relative to one, some or all other analytes in the sample. Moreover, binding specificity may refer to selectivity for a subset of analytes in a given sample relative to at least one other analyte in the sample.

In some of the implementations described herein, the term “click reaction” can refer to single-step, thermodynamically-favorable conjugation reaction utilizing biocompatible reagents. A click reaction may be configured to not utilize toxic or biologically incompatible reagents (e.g., acids, bases, heavy metals) or to not generate toxic or biologically incompatible byproducts. A click reaction may utilize an aqueous solvent or buffer (e.g., phosphate buffer solution, Tris buffer, saline buffer, MOPS, etc.). A click reaction may be thermodynamically favorable if it has a negative Gibbs free energy of reaction, for example a Gibbs free energy of reaction of less than about −5 kiloJoules/mole (kJ/mol), −10 kJ/mol, −25 kJ/mol, −100 kJ/mol, −250 kJ/mol, −500 kJ/mol, or less. Exemplary click reactions may include metal-catalyzed azide-alkyne cycloaddition, strain-promoted azide-alkyne cycloaddition, strain-promoted azide-nitrone cycloaddition, strained alkene reactions, thiol-ene reaction, Diels-Alder reaction, inverse electron demand Diels-Alder reaction (IEDDA), [3+2] cycloaddition, [4+1] cycloaddition, nucleophilic substitution, dihydroxylation, thiol-yne reaction, photoclick, nitrone dipole cycloaddition, norbornene cycloaddition, oxanobornadiene cycloaddition, tetrazine ligation, and tetrazole photoclick reactions. Exemplary reactive moieties utilized to perform click reactions may include alkenes, alkynes, azides, epoxides, amines, thiols, nitrones, isonitriles, isocyanides, aziridines, activated esters, and tetrazines. Other well-known click conjugation reactions may be used having complementary bioorthogonal reaction species, for example, where a first click component comprises a hydrazine moiety and a second click component comprises an aldehyde or ketone group, and where the product of such a reaction comprises a hydrazone functional group or equivalent. Exemplary bioorthogonal and click reactions are set forth in US Pat. App. Pub. No. 2021/0101930 A1, which is incorporated herein by reference.

The term “comprising” is intended herein to be open-ended, including not only the recited elements, but further encompassing any additional elements.

As used herein, the term “each,” when used in reference to a collection of items, is intended to identify an individual item in the collection but does not necessarily refer to every item in the collection. Exceptions can occur if explicit disclosure or context clearly dictates otherwise.

In some of the implementations described herein, the term “epitope” can refer to an affinity target within a protein, polypeptide or other analyte. Epitopes may include amino acid sequences that are sequentially adjacent in the primary structure of a protein. Epitopes may include amino acids that are structurally adjacent in the secondary, tertiary or quaternary structure of a protein despite being non-adjacent in the primary sequence of the protein. An epitope can be, or can include, a moiety of protein that arises due to a post-translational modification, such as a phosphate, phosphotyrosine, phosphoserine, phosphothreonine, or phosphohistidine. An epitope can optionally be recognized by or bound to an antibody. However, an epitope need not necessarily be recognized by any antibody, for example, instead being recognized by an aptamer, mini-protein or other affinity reagent. An epitope can optionally bind an antibody to elicit an immune response. However, an epitope need not necessarily participate in, nor be capable of, eliciting an immune response.

In some of the implementations described herein, the term “exogenous,” when used in reference to a moiety of a molecule, can mean that the moiety is not present in a natural analog of the molecule. For example, an exogenous label of an amino acid is a label that is not present on a naturally occurring amino acid. Similarly, an exogenous label that is present on an antibody is not found on the antibody in its native milieu.

In some of the implementations described herein, the term “fluid phase,” when used in reference to a molecule, can mean that the molecule is in a state wherein it is mobile in a fluid, for example, being capable of diffusing through the fluid.

In some of the implementations described herein, the terms “group” and “moiety” are intended to be synonymous when used in reference to the structure of a molecule. The terms refer to a component or part of the molecule. The terms do not necessarily denote the relative size of the component or part compared to the rest of the molecule, unless indicated otherwise.

In some of the implementations described herein, the term “identifier moiety” can refer to a moiety that is joined to an analyte or a binding reagent, and that facilitates the formation of an interaction identification moiety during a binding interaction. An identifier moiety may contain information, such as a code or residue sequence, that is incorporated into an interaction identification moiety. An identifier moiety may contain information that is transferred to another moiety during the recording of a binding interaction. An identifier moiety may alter or transform another entity, such as another identifier moiety, thereby recording a binding interaction. An identifier moiety may comprise a polymer strand containing a unique sequence of residues. Exemplary polymer strands that can be formed into an identifier moiety can include nucleic acid strands, peptide strand, polysaccharide strands, and combinations thereof. In some of the implementations described herein, the term “analyte identifier moiety” can refer to an identifier moiety that is associated with or joined to an analyte during a binding interaction. An analyte identifier moiety can contain an analyte-specific code or residue sequence that will only be detected in interaction identification moieties derived from binding interactions of the associated or joined analyte. In some of the implementations described herein, the term “binding reagent identifier moiety” or “affinity reagent identifier moiety” can refer to an identifier moiety that is associated with or joined to a binding reagent or affinity reagent, respectively, during a binding interaction. A binding reagent identifier moiety or affinity reagent identifier moiety can contain a binding reagent-specific code, affinity reagent-specific code, or residue sequence that will only be detected in interaction identification moieties derived from binding interactions of the associated or joined analyte.

In some of the implementations described herein, the term “interaction identification moiety” can refer to a detectable moiety that is formed during the recording of a binding interaction. An interaction identification moiety can comprise an analyte-specific code or residue sequence that identifies the analyte that participated in the recorded binding reaction. An interaction identification moiety can further comprise one or more codes or residue sequences (e.g., a binding-reagent specific code, an assay sequence-specific code, a vessel-specific code) that provides information regarding the binding reagent that participated in the recorded binding reaction, or the time and/or location when the recorded binding interaction occurred.

In some of the implementations described herein, the term “joined,” when used in reference to an identifier moiety, can refer to the identifier moiety being co-located with an entity such as a binding reagent or an analyte. An identifier moiety may be joined to an entity if the identifier moiety is attached to the entity (e.g., covalently attached, non-covalently attached). An identifier may be joined to an entity if the identifier moiety and the entity are each attached to a second entity (e.g., a particle, a solid support). If an identifier moiety is joined to an entity, the identifier moiety can be present and within a close proximity to the entity regardless of the spatial position or movement of the entity.

In some of the implementations described herein, the term “linker” can refer to a moiety that connects two objects to each other. One or both objects can be a molecule, solid support, address, particle or bead. Both objects can be moieties of a molecule, solid support, address, particle or bead. The term can also refer to an atom, moiety or molecule that is configured to react with two objects to form a moiety that connects the two objects. The connection of a linker to one or both objects can be a covalent bond or non-covalent bond. A linker may be configured to provide a chemical or mechanical property to the moiety connecting two objects, such as hydrophobicity, hydrophilicity, electrical charge, polarity, rigidity, or flexibility. A linker may comprise two or more functional groups that facilitate coupling of the linker to the first and second objects. A linker may include a polyfunctional linker such as a homobifunctional linker, heterobifunctional linker, homopolyfunctional linker, or heteropolyfunctional linker. Exemplary compositions for linkers can include, but are not limited to, a polyethylene glycol (PEG), polyethylene oxide (PEO), amino acid, protein, nucleotide, nucleic acid, nucleic acid origami, dendrimer, protein nucleic acid (PNA), polysaccharide, carbon, nitrogen, oxygen, ether, sulfur, or disulfide. A linker can be a bead or particle such as a structured nucleic acid particle.

In some of the implementations described herein, the term “measurement outcome” can refer to information resulting from an observation, simulation or examination of a process. For example, the measurement outcome for contacting an affinity reagent with an analyte can be referred to as a “binding outcome.” A measurement outcome can be positive or negative. For example, observation of binding is a positive binding outcome and observation of non-binding is a negative binding outcome. A measurement outcome can be a null outcome in the event that a positive or negative outcome is not apparent from a given measurement. An “empirical” measurement outcome includes information based on observation of a signal from an analytical technique. A “putative” measurement outcome includes information based on theoretical or a priori evaluation of an analytical technique or analytes. A “candidate” measurement outcome includes an empirical or putative measurement outcome for a candidate analyte (e.g. for a candidate protein) that is known or suspected of being present in a sample or assay. A measurement outcome can be represented in binary terms, such as a zero (0) for a negative binding outcome and a one (1) for a positive binding outcome. In some cases a ternary representation can be used, for example, when zero (0) represents a negative binding outcome, one (1) represents a positive binding outcome, and two (2) represents a null outcome. It is also possible to use continuous or analog values, as opposed to integers or discrete values, to represent different measurement outcomes.

In some of the implementations described herein, the term “nucleic acid origami” can refer to a nucleic acid construct having an engineered tertiary or quaternary structure. A nucleic acid origami may include DNA, RNA, PNA, modified or non-natural nucleic acids, or combinations thereof. A nucleic acid origami may include a plurality of oligonucleotides that hybridize via sequence complementarity to produce the engineered structuring of the origami. A nucleic acid origami may include sections of single-stranded or double-stranded nucleic acid, or combinations thereof. Exemplary nucleic acid origami structures may include nanotubes, nanowires, cages, tiles, nanospheres, blocks, and combinations thereof. A nucleic acid origami can optionally include a relatively long scaffold nucleic acid to which multiple smaller nucleic acids hybridize, thereby creating folds and bends in the scaffold that produce an engineered structure. The scaffold nucleic acid can be circular or linear. The scaffold nucleic acid can be single stranded but for hybridization to the smaller nucleic acids. A smaller nucleic acid (sometimes referred to as a “staple”) can hybridize to two regions of the scaffold, wherein the two regions of the scaffold are separated by an intervening region that does not hybridize to the smaller nucleic acid.

In some of the implementations described herein, the term “post-translational modification” can refer to a change to the chemical composition of a protein compared to the chemical composition encoded by the gene for the protein. Exemplary changes include those that alter the presence, absence or relative arrangement of different regions of amino acid sequence (e.g., splicing variants, or protein processing variants of a single gene), or due to the presence or absence of different moieties on particular amino acids (e.g., post-translationally modified variants of a single gene). A post-translational modification can be derived from an in vivo process or an in vitro process. A post-translational modification can be derived from a natural process or a synthetic process. Exemplary post-translational modifications include those classified by the PSI-MOD ontology. See Smith, L. M. et al. Nat. Methods, 2013, 10, 186-187.

In some of the implementations described herein, the term “promiscuous,” when used in reference to a reagent, can mean that the reagent is known or suspected to react with a variety of different analytes in a given sample. For example, an affinity reagent that is known or suspected to recognize a variety of different analytes (e.g. a variety of proteins having different primary sequences) is promiscuous. A promiscuous reagent may be known or suspected of having high reactivity with one or more of the different analytes with which it reacts. For example, a promiscuous affinity reagent may have high affinity for one or more of the different analytes that it recognizes. A promiscuous reagent may be composed of a single species of reagent, such as a single affinity reagent, or a promiscuous reagent may be composed of two or more different species of reagent. For example, a promiscuous affinity reagent may be composed of a single species of antibody that recognizes a variety of different proteins in a sample, or the promiscuous affinity reagent may be composed of a pool containing several different antibody species that collectively recognize the variety of different proteins in the sample.

In some of the implementations described herein, the term “protein” can refer to a molecule comprising two or more amino acids joined by a peptide bond. A protein may also be referred to as a polypeptide, oligopeptide or peptide. A protein can be a naturally-occurring molecule, or synthetic molecule. A protein may include one or more non-natural amino acids, modified amino acids, or non-amino acid linkers. A protein may contain D-amino acid enantiomers, L-amino acid enantiomers or both. Amino acids of a protein may be modified naturally or synthetically, such as by post-translational modifications. A protein may have a known biological activity or function. A protein can refer to a full-length or intact sequence of amino acids, as translated from a gene of an organism, or a spliced variant thereof. In some circumstances, different proteins may be distinguished from each other based on different genes from which they are expressed in an organism, different primary sequence length or different primary sequence composition. Proteins expressed from the same gene may nonetheless be different proteoforms, for example, being distinguished based on non-identical length, non-identical amino acid sequence or non-identical post-translational modifications. Different proteins can be distinguished based on one or both of the gene of origin and the proteoform state. In some of the implementations described herein, the term “peptide” may refer to a short polypeptide (e.g., containing no more than about 50, 40, 30, 25, 20, 15, 10, or less than 10 amino acid residues). A peptide may be a fragment of a full-length protein. A peptide may be naturally-occurring or synthetic. A peptide may have a biological activity or function.

In some of the implementations described herein, the term “recording,” when used in reference to a binding interaction, can refer to a detectable moiety being formed due to the binding interaction. A binding interaction may be recorded if an interaction identification moiety is formed. The detectable moiety formed by a recording event may be a non-transitory moiety. The detectable moiety formed by a recording event may be detectable after intermediate steps occur between the formation of the detectable moiety and a detection event for the detectable moiety.

In some of the implementations described herein, the term “single,” when used in reference to an object such as an analyte, can mean that the object is individually manipulated or distinguished from other objects. A single analyte can be a single molecule (e.g. single protein), a single complex of two or more molecules (e.g. a multimeric protein having two or more separable subunits, a single protein attached to a structured nucleic acid particle or a single protein attached to an affinity reagent), a single particle, or the like. Reference herein to a “single analyte” in the context of a composition, system or method herein does not necessarily exclude application of the composition, system or method to multiple single analytes that are manipulated or distinguished individually, unless indicated contextually or explicitly to the contrary.

In some of the implementations described herein, the term “single-analyte resolution” can refer to the detection of, or ability to detect, an analyte on an individual basis, for example, as distinguished from its nearest neighbor in an array.

In some of the implementations described herein, the term “solid support” can refer to a substrate that is insoluble in aqueous liquid. Optionally, the substrate can be rigid. The substrate can be non-porous or porous. The substrate can optionally be capable of taking up a liquid (e.g. due to porosity) but will typically, but not necessarily, be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers. In particular configurations, a flow cell contains the solid support such that fluids introduced to the flow cell can interact with a surface of the solid support to which one or more components of a binding event (or other reaction) is attached.

In some of the implementations described herein, the term “structured nucleic acid particle” or “SNAP” can refer to a single- or multi-chain polynucleotide molecule having a compacted three-dimensional structure. The compacted three-dimensional structure can optionally be characterized in terms of hydrodynamic radius or Stoke's radius of the SNAP relative to a random coil or other non-structured state for a nucleic acid having the same sequence length as the SNAP. The compacted three-dimensional structure can optionally be characterized with regard to tertiary structure. For example, a SNAP can be configured to have an increased number of internal binding interactions between regions of a polynucleotide strand, less distance between the regions, increased number of bends in the strand, and/or more acute bends in the strand, as compared to a nucleic acid molecule of similar length in a random coil or other non-structured state. Alternatively or additionally, the compacted three-dimensional structure can optionally be characterized with regard to tertiary or quaternary structure. For example, a SNAP can be configured to have an increased number of interactions between polynucleotide strands or less distance between the strands, as compared to a nucleic acid molecule of similar length in a random coil or other non-structured state. In some configurations, the secondary structure of a SNAP can be configured to be more dense than a nucleic acid molecule of similar length in a random coil or other non-structured state. A SNAP may contain DNA, RNA, PNA, modified or non-natural nucleic acids, or combinations thereof. A SNAP may include a plurality of oligonucleotides that hybridize to form the SNAP structure. The plurality of oligonucleotides in a SNAP may include oligonucleotides that are attached to other molecules (e.g., probes, analytes such as proteins, reactive moieties, or detectable labels) or are configured to be attached to other molecules (e.g., by functional groups). A SNAP may include engineered or rationally designed structures. Exemplary SNAPs include nucleic acid origami and nucleic acid nanoballs.

In some of the implementations described herein, the term “tag” can refer to a polymer sequence, such as a nucleic acid sequence or peptide sequence, that is encoded with information that uniquely identifies an object with which it is associated. A tag can be associated with an object via a connection. The connection can be physical, including for example, attachment, colocalization, diffusional contact or the like. Non-physical connections can include, for example, knowledge of a past interaction, knowledge of a shared characteristic, knowledge of common manipulations, knowledge of origin or the like. The tag can be, for example, DNA, RNA, peptides, polysaccharides, or analogs thereof. The length of the tag sequence can be at least about 5, 8, 10, 15, 20, 25, 30, 40, 50, 75, 100 or more residues. Alternatively or additionally, the length of the tag sequence can be at most about 100, 75, 50, 40, 30, 25, 20, 15, 10, 8, 5 or fewer residues.

In some of the implementations described herein, the term “type,” when used in reference to a subset of analytes, can refer to a characteristic that is shared by the analytes in the subset and that distinguishes the analytes in the subset from analytes that are not in the subset. The characteristic can be any of a variety of characteristics known for the analytes. Any of a variety of analytes can be categorized by type, including for example, proteins. Exemplary characteristics that can be used to categorize proteins by type include, but are not limited to, amino acid composition, full length amino acid sequence, proteoform, presence or absence of an amino acid sequence motif, number of amino acids present (i.e. sequence length), molecular weight, presence or absence of a particular epitope, presence or absence of epitope(s) recognized by a particular affinity reagent, probability of binding a particular affinity reagent, presence or absence of a post-translational modification, enzymatic activity, affinity for binding a particular protein or protein motif, or the like.

In some of the implementations described herein, the term “unique identifier” can refer to a moiety, object or substance that is associated with an analyte and that is distinct from other identifiers, throughout one or more steps of a process. The moiety, object or substance can be, for example, a solid support such as a particle or bead; a location on a solid support; a spatial address in an array; a tag; a label such as a luminophore; a molecular barcode such as a nucleic acid having a unique nucleotide sequence or a protein having a unique amino acid sequence; or an encoded device such as a radiofrequency identification (RFID) chip, electronically encoded device, magnetically encoded device or optically encoded device. The process in which a unique identifier is used can be an analytical process, such as a method for detecting, identifying, characterizing or quantifying an analyte; a separation process in which at least one analyte is separated from other analytes; or a synthetic process in which an analyte is modified or produced. The unique identifier can be associated with an analyte via immobilization. For example, a unique identifier can be covalently or non-covalently (e.g. ionic bond, hydrogen bond, van der Waals forces etc.) attached to an analyte. A unique identifier can be exogenous to an associated analyte, for example, being synthetically attached to the associated analyte. Alternatively, a unique identifier can be endogenous to the analyte, for example, being attached or associated with the analyte in the native milieu of the analyte.

In some of the implementations described herein, the term “unique identifier label” can refer to a unique identifier that is a particle, molecule or moiety that provides a detectable characteristic. The detectable characteristic can be, for example, an optical signal such as absorbance of radiation, luminescence (e.g. fluorescence) emission, luminescence lifetime, luminescence polarization, or the like; Rayleigh and/or Mie scattering; binding affinity for a ligand or receptor; magnetic properties; electrical properties; charge; mass; radioactivity or the like. Exemplary labels include, without limitation, a fluorophore, luminophore, chromophore, nanoparticle (e.g., gold, silver, carbon nanotubes), heavy atoms, radioactive isotope, mass label, charge label, spin label, receptor, ligand, or the like.

In some of the implementations described herein, the term “vessel” can refer to an enclosure that contains a substance. The enclosure can be permanent or temporary with respect to the timeframe of a method set forth herein or with respect to one or more steps of a method set forth herein. Exemplary vessels include, but are not limited to, a well (e.g. in a multiwell plate or array of wells), test tube, channel, tubing, pipe, flow cell, bottle, vesicle, droplet that is immiscible in a surrounding fluid, or the like. A vessel can be entirely sealed to prevent fluid communication from inside to outside, and vice versa. Alternatively, a vessel can include one or more ingress or egress to allow fluid communication between the inside and outside of the vessel. A vessel can be made from multiple materials, for example, including a well in a solid support that is covered by a seal such as a wax or fluid that is immiscible with the fluid in the well.

The embodiments set forth below and recited in the claims can be understood in view of the above definitions.

### Methods and Systems for Forming Binding Profiles

Methods and systems provided herein may facilitate recording of a binding interaction between a single analyte and a binding reagent. Preferably, the binding interaction may occur under an equilibrium binding condition. The binding interaction between the analyte and the binding reagent can be detected by a non-transitory moiety that remain detectable even if a complex containing the analyte and the binding reagent dissociates. The methods and systems set forth herein can be extended to recording binding interaction between populations of analytes and pluralities of binding reagents. Incorporation of an analyte-specific code into each recorded non-transitory moiety can facilitate discrimination of which single analytes formed binding interactions with binding reagents. Advantageously, the methods and systems set forth herein can be provided without single-analyte optical resolution of individual analytes.

Provided herein is a method of observing a binding interaction between a binding reagent and an analyte, comprising: (a) binding a binding reagent to an analyte, wherein the binding reagent is joined with a binding reagent identifier moiety, and wherein the analyte is joined with an analyte identifier moiety, wherein the analyte identifier moiety comprises an analyte-specific code, (b) after binding the binding reagent to the analyte, forming an interaction identification moiety, wherein the interaction identification moiety comprises a binding reagent identifier tag transferred from the binding reagent identifier moiety and the analyte-specific code from the analyte identifier moiety, and (c) detecting the analyte-specific code of the interaction identification moiety, thereby detecting the binding interaction between the binding reagent and the analyte. In some configurations, the binding reagent may comprise an epitope-binding affinity reagent (e.g., an antibody, an antibody fragment, an aptamer, a peptamer, an avimer, etc.).

Although aspects of detecting a binding interactions between analytes and binding reagents have been described with respect to a single analyte, the methods and systems are readily extendable to populations of analytes, including populations of analytes containing two or more different species of analyte (e.g., as characterized by primary structure; as characterized by proteoform or other chemical modification, etc.). The skilled person will readily recognize that a binding reagent may be more likely to form a binding interaction with a first species of analyte and less likely to form a binding interaction with a second species of analyte. Accordingly, detection of an interaction identification moiety may provide evidence that the first species of analyte is present in a sample suspected of containing both the first species of analyte and the second species of analyte. The methods set forth herein may readily be extended to facilitate identification, characterization, and/or quantitation of two or more species of analyte in a population of analytes by differences in binding behavior between the two or more species of analytes.

Provided herein is a method of forming a binding profile, comprising: (a) binding reagents to analytes of a plurality of analytes, wherein each of the binding reagents is individually joined with a binding reagent identifier moiety, and wherein each of the analytes is individually joined with a unique analyte identifier moiety (e.g., each unique analyte identifier moiety comprising a unique analyte-specific code), (b) for each analyte bound to a binding reagent, coupling together the binding reagent identifier moiety and the unique analyte identifier moiety, thereby forming an interaction identification moiety comprising an analyte-specific code from the unique analyte identifier moiety, (c) repeating steps (a) and (b) at least once with differing binding reagents, (d) detecting for each interaction identification moiety from steps (b) and (c) the analyte-specific code, and (e) for each analyte of the plurality of analytes, forming a binding profile for the analyte, wherein the binding profile comprises a presence or absence of a detected analyte-specific code for the analyte for each of the at least two differing binding reagents. A method may further comprise, based upon binding profiles of the analytes of the plurality of analytes, identifying the presence of the two or more species of analytes in the plurality of analytes.

A population of analytes may comprise at least about 2, 5, 10, 50, 100, 500, 1000, 2000, 5000, 10000, 20000, 30000, 50000, 100000, 1000000, or more than 1000000 species of analytes. Alternatively or additionally, a population of analytes may comprise no more than about 1000000, 100000, 50000, 30000, 20000, 10000, 5000, 2000, 1000, 500, 100, 50, 10, 5, 2, or 1 species of analytes.

Methods set forth herein may be utilized to characterize analytes of a population of analytes. A method may comprise characterizing at least about 10⁶, 10⁷, 10⁸, 10⁹, 10¹⁰, 10¹¹, 10¹², or more than 10¹² analytes. A population of analytes may be provided in a vessel. In some cases, a population of analytes may be divided into a plurality of vessels. A method may comprise characterizing a population of analytes or a subpopulation thereof in a vessel, in which the vessel comprises at least about 10⁶, 10⁷, 10⁸, 10⁹, 10¹⁰, 10¹¹, 10¹², or more than 10¹² analytes.

In some cases, a population of analytes may be provided, in which the population of analytes comprises a first species of analyte and a second species of analyte, in which the first species and the second species have a dynamic range of at least about 10⁴ (e.g., at least about 10⁵, 10⁶, 10⁷, 10⁸, 10⁹, 10¹⁰, or more than 10¹⁰). A method may further comprise, based upon binding profiles of the analytes of the plurality of analytes, identifying the presence of the first species of analyte and the second species of analyte in the plurality of analytes having the dynamic range of at least about 10⁴. A method may further comprise determining a quantity of the first species of analyte and the second species of analyte in the plurality of analytes in the plurality of analytes having the dynamic range of at least about 10⁴.

In some configurations, a population of analytes may comprise a population of proteins. A population of proteins may comprise two or more species of proteins, as determined by full-length primary amino acid structure. A population of proteins may comprise a population of intact proteins (e.g., proteins not fragmented into smaller peptides by an endogenous or exogenous protease). A population of proteins may comprise two or more species of proteins, as determined by proteoform or isoform. For example, a population of proteins may comprise two or more proteoforms or isoforms of a single species of protein. A population of proteins may comprise one or more artificial or synthetic proteins. A population of proteins may comprise one or more proteins produced by a recombinant organism. In some cases, a method may comprise providing a population of proteins, in which the proteins are provided in a denatured or partially-denatured state. A method may further comprise contacting binding reagents to the population of proteins, in which the proteins are in a partially- or fully denatured state. In other cases, a method may comprise providing a population of proteins, in which the proteins are provided in their native conformations. A method may further comprise contacting binding reagents to the population of proteins, in which the proteins are in their native conformations.

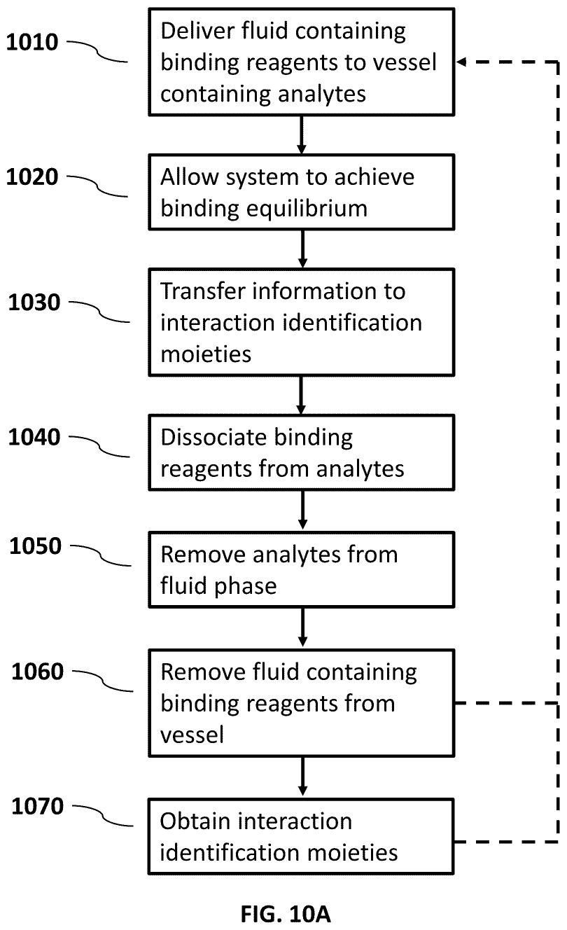

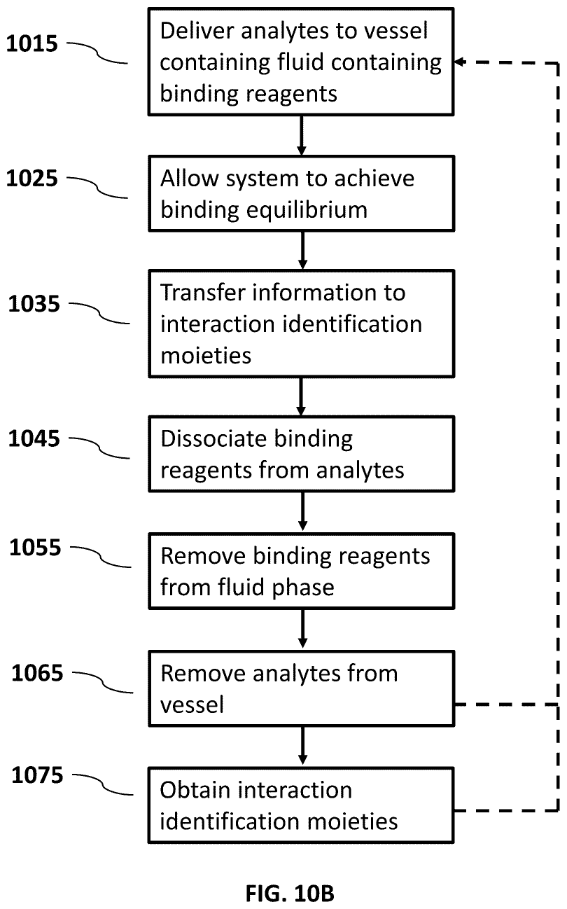

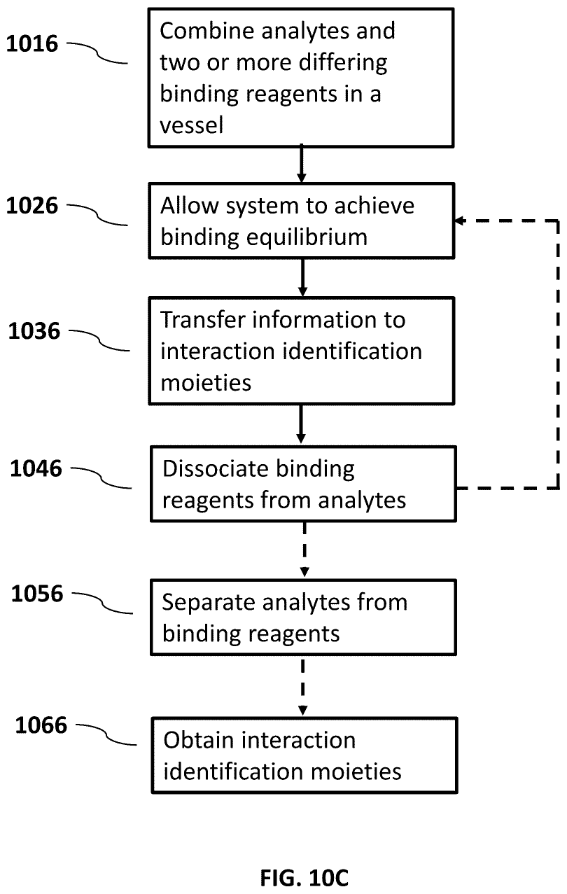

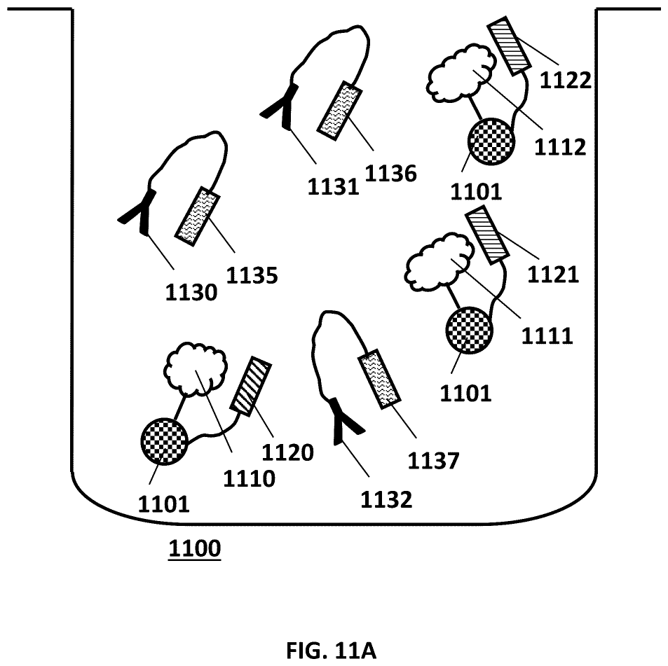

Numerous methods, compositions, and systems can be conceived based upon the present disclosure. FIGS. 10A-10C provide schematics of methods of characterizing one or more analytes with binding reagents. FIG. 10A provides a method for characterizing analytes in a vessel, in which binding reagents are delivered to the vessel containing the analytes. In a first step 1010, a fluid containing binding reagents is delivered to the vessel containing one or more analytes. In a second step 1020, the system is allowed to equilibrate, thereby establishing a binding equilibrium between the binding reagents and the one or more analytes. In a third step 1030, information is transferred into an interaction identification moiety, as set forth herein, for any single analyte that is complexed with a binding reagent, in which the formed interaction identification moiety contains a unique identifier for the single analyte and a unique identifier for the binding reagent. In a fourth step 1040, binding reagents are dissociated from the one or more analytes (e.g., by heating, by introducing a dissociation medium, etc.). In a fifth step 1050, analytes are removed from a fluid phase containing the binding reagents (e.g., by centrifugation, sedimentation, magnetic separation, or charge-based separation). In a sixth step 1060, the fluid containing the binding reagents is removed from the vessel. Optionally, steps 1010 through 1060 are repeated one or more times with a substantially identical or differing set of binding reagents. In a seventh step 1070, interaction identification moieties are obtained by dissociation from analytes, particles, or binding reagents. Obtaining interaction identification moieties may include steps of delivering a fluid that is devoid of interaction identification moieties to the vessel and removing a fluid containing the interaction identification moieties from the vessel after dissociation. Optionally, interaction identification moieties may be obtained after each contact between binding reagents and the one or more analytes and before a new set of binding reagents is delivered to the vessel.

FIG. 10B provides a schematic for a similar method to the method of FIG. 10A, with analytes delivered to a vessel rather than the binding reagents. In a first step 1015, a fluid containing one or more analytes is delivered to the vessel containing a set of binding reagents. In a second step 1025, the system is allowed to equilibrate, thereby establishing a binding equilibrium between the binding reagents and the one or more analytes. In a third step 1035, information is transferred into an interaction identification moiety, as set forth herein, for any single analyte that is complexed with a binding reagent, in which the formed interaction identification moiety contains a unique identifier for the single analyte and a unique identifier for the binding reagent. In a fourth step 1045, binding reagents are dissociated from the one or more analytes (e.g., by heating, by introducing a dissociation medium, etc.). In a fifth step 1055, binding reagents are removed from a fluid phase containing the binding reagents (e.g., by centrifugation, sedimentation, magnetic separation, or charge-based separation). In a sixth step 1065, the fluid containing the one or more analytes is removed from the vessel. Optionally, steps 1015 through 1065 are repeated one or more times with a different one or more analytes. In a seventh step 1075, interaction identification moieties are obtained by dissociation from analytes, particles, or binding reagents. Obtaining interaction identification moieties may include steps of delivering a fluid that is devoid of interaction identification moieties to the vessel and removing a fluid containing the interaction identification moieties from the vessel after dissociation. Optionally, interaction identification moieties may be obtained after each contact between binding reagents and the one or more analytes and before new analytes are delivered to the vessel.

FIG. 10C depicts a schematic of a “one-pot” method that facilitates multiplexing of differing binding reagents within a single vessel. In a first step 1016, one or more analytes are combined with two or more differing sets of binding reagents in a vessel. In a second step 1026, the system is allowed to equilibrate, thereby establishing a binding equilibrium between the binding reagents and the one or more analytes. In a third step 1036, information is transferred into an interaction identification moiety, as set forth herein, for any single analyte that is complexed with a binding reagent, in which the formed interaction identification moiety contains a unique identifier for the single analyte and a unique identifier for the binding reagent. In a fourth step 1046, binding reagents are dissociated from the one or more analytes (e.g., by heating, by introducing a dissociation medium, etc.). Optionally, steps 1026 through 1046 are repeated one or more times (e.g., by temperature modulation, by pH modulation, etc.). Preferably, no additional analytes or binding reagents are introduced to the vessel during the repetition of binding events. Optionally, in a fifth step 1056, binding reagents are separated from analytes. Separation may include removing analytes or binding reagents from the vessel, such as by methods described in FIGS. 10A-10B. In a seventh step 1066, interaction identification moieties are obtained by dissociation from analytes, particles, or binding reagents.

The methods of FIGS. 10A-10C are readily multiplexed. For example, a plurality of vessels can be provided (e.g., a multiwell plate), in which each vessel retains a set of one or more analytes (as per FIG. 10A) or a set of binding reagents (as per FIG. 10B). In this configuration, fluids containing analytes or binding reagents may be transferred from vessel to vessel, then the method repeated. Further, the “one-pot” method of FIG. 10C may be multiplexed by the transfer of analytes or sets of two or more differing binding reagents from vessel to vessel. Although certain figures provided herein depict attachment of analytes to particles that facilitate separation from the fluid phase, if a method is configured to retain binding reagents within a vessel (e.g., FIG. 10B), it may be preferable to provide binding reagents and associated affinity reagent identifier moieties attached to particles that facilitate separation from a fluid phase.

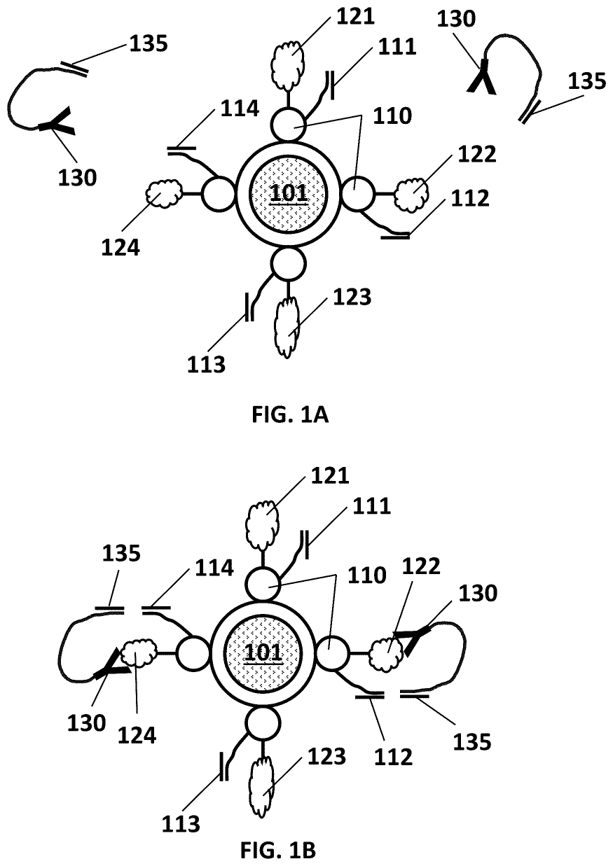

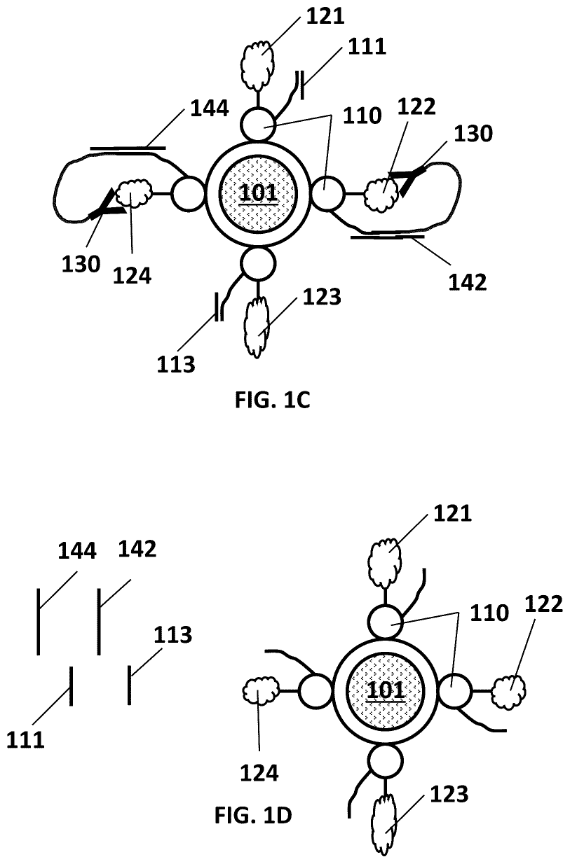

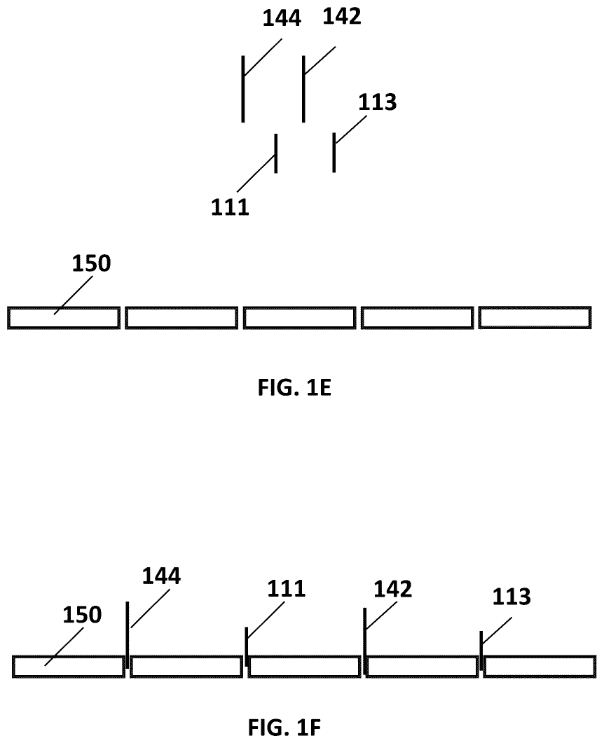

FIGS. 1A-IF illustrate an embodiment of a method of recording binding interactions between a plurality of binding reagents and a population of analytes. FIG. 1A depicts a population of analytes (121, 122, 123, 124) that are attached to a solid support 101 (e.g., a bead, a particle, an array, etc.). Each single analyte is attached to a particle 110 that mediates attachment of the analytes to the solid support 101. The particles 110 also join or co-locate with each single analyte an analyte identifier moiety (111, 112, 113, 114) containing an analyte-specific code that is unique for each analyte (i.e., the analyte-specific code of analyte identifier moiety 111 differs from the analyte-specific code of analyte identifier moiety 112, 113, and 114). The solid support 101 and/or analytes (111, 112, 113, 114) are contacted with a plurality of binding reagents 130 and an optional binding reagent identifier moiety 135. FIG. 1B depicts a second configuration, in which binding reagents 130 have bound to analytes 122 and 124. The association of the binding reagents to analytes 122 and 124 bring the binding reagent identifier moieties 135 into close proximity of analyte identifier moieties 112 and 114. FIG. 1C depicts a third configuration, in which interaction identification moieties (142, 144) for the binding interactions between binding reagents 130 and analytes 122 and 124 have been formed by attaching binding reagent identifier moieties 135 to analyte identifier moieties (112, 114). FIG. 1D depicts a fourth configuration, in which the binding reagents have been dissociated from analytes 122 and 124, thereby providing the solid support 101 attached to the analytes. Interaction identification moieties (142, 144) and analyte identifier moieties (111, 113) have also been dissociated from the solid support 101.

FIGS. 1E and 1F depict subsequent steps of detecting presence or absence of binding interactions for each analyte (111, 112, 113, 114) that was contacted with a binding reagent. As shown in FIG. 1E, the pool of moieties (111, 113, 142, 144) are delivered to a detection device such as a sequencing device 150 (e.g., a nucleic acid sequencer, a peptide sequencer, etc.). FIG. 1F depicts the detection of the pool of moieties, thereby identifying presence or absence of a binding interaction for each analyte attached to the solid support 101. In this configuration, presence of a binding interaction for a single analyte can be determined by detecting a binding reagent-specific code paired with an analyte-specific code when an interaction identification moiety (142, 144) is sequenced. Otherwise, only an analyte-specific code of an analyte identifier moiety (111, 113) will be detected, thereby detecting absence of the binding interaction.

Methods and systems of the present disclosure may be provided with binding reagents that are configured to facilitate recording and detection of binding reactions between the binding reagents and binding targets such as analytes. A binding reagent may be joined to a binding reagent identifier moiety, in which the binding reagent identifier moiety facilitates recording and detection of binding reactions of the binding reagent. A binding reagent identifier moiety may comprise one or more codes or sequences that provide information about the binding reagent. For example, a binding reagent identifier moiety may comprise a binding reagent-specific code that can be correlated to the binding specificity of the binding reagent. In another example, a binding reagent identifier moiety may comprise a vessel-specific code that can identify a vessel in which the binding reagent was utilized. In another example, a binding reagent identifier moiety may comprise an assay sequence-specific code that can be correlated to the time or step in an assay at which the binding reagent was utilized. Polymeric strands (e.g., polynucleotides, polypeptides, polysaccharides) may be particularly useful for forming binding reagent identifier moieties because polymeric residues or monomers can be ordered into unique sequences with high plexity (i.e., a nucleic acid strand with N residues can produce 4^{N} unique sequences of nucleotides; a peptide strand with N residues can produce 20^{N} unique sequence of amino acids). A method or system set forth herein may form an interaction identification moiety, in which the interaction identification moiety comprises information provided by or transferred from a binding reagent identifier moiety (e.g., a binding reagent-specific code, an assay sequence-specific code).

A binding reagent identifier moiety may be joined to a binding reagent. In some configurations, a binding reagent may be joined to a plurality of binding reagent identifier moieties. In particular configurations, a binding reagent may be joined to a plurality of binding reagent identifier moieties, each binding reagent identifier moiety comprising an identical code or sequence (e.g., a binding reagent-specific code, an assay sequence-specific code). In some configurations, a binding reagent identifier moiety may be attached to a binding reagent. For example, a binding reagent may be covalently attached to a binding reagent identifier moiety (e.g., a polymeric strand). In some configurations, a binding reagent identifier moiety and a binding reagent may be joined by a retaining component. A retaining component could comprise a linking moiety (e.g., a polymer strand) that attaches the binding reagent identifier moiety to the binding reagent. A retaining component could comprise a particle (e.g., an inorganic nanoparticle, a carbon nanoparticle, a nucleic acid nanoparticle, a polypeptide particle) that is attached to the binding reagent identifier moiety and the binding reagent.

Methods and systems of the present disclosure may be provided with analytes that are configured to facilitate recording and detection of binding reactions between binding reagents and analytes. An analyte may be joined to an analyte identifier moiety, in which the analyte identifier moiety facilitates recording and detection of binding reactions of the analyte. An analyte identifier moiety may comprise one or more codes or sequences that provide information about the analyte. For example, an analyte identifier moiety may comprise an analyte-specific code that can be correlated to the binding interactions of a single analyte throughout an assay that may comprise multiple opportunities for the single analyte to form binding interactions. Polymeric strands (e.g., polynucleotides, polypeptides, polysaccharides) may be particularly useful for forming analyte identifier moieties because polymeric residues or monomers can be ordered into unique sequences with high plexity. A method or system set forth herein may form an interaction identification moiety, in which the interaction identification moiety comprises information provided by or transferred from an analyte identifier moiety (e.g., an analyte-specific code).

An analyte identifier moiety may be joined to an analyte. In some configurations, an analyte may be joined to a plurality of analyte identifier moieties. In particular configurations, an analyte may be joined to a plurality of analyte identifier moieties, each analyte identifier moiety comprising an identical analyte-specific code. In some configurations, an analyte identifier moiety may further comprise a vessel-specific code that can identify a vessel in which the analyte was contained. In some configurations, an analyte identifier moiety may be attached to an analyte. For example, an analyte (e.g., a polypeptide, a nucleic acid, a polysaccharide) may be covalently attached to an analyte identifier moiety (e.g., a polymeric strand). In some configurations, an analyte identifier moiety and an analyte may be joined by a retaining component. A retaining component could comprise a linking moiety (e.g., a polymer strand) that attaches the analyte identifier moiety to the analyte. A retaining component could comprise a particle (e.g., an inorganic nanoparticle, a carbon nanoparticle, a nucleic acid nanoparticle, a polypeptide particle) that is attached to the analyte identifier moiety and the analyte. It may be useful to attach a single analyte and an analyte identifier moiety to a retaining component to maintain co-localization of the single analyte with its associated analyte identifier moiety throughout an assay.

A single analyte and an associated analyte identifier moiety may be co-located on a solid support. In some configurations, the single analyte and the associated analyte identifier moiety may be attached to a particle, in which the particle is attached to a solid support. A solid support for attachment of analytes and co-located analyte identifier moieties can include an array (e.g., a patterned array, an unpatterned array). Alternatively, a solid support may comprise one or more beads or particles. Bead or particle solid supports may be advantageous as they can facilitate mixing and contact between analytes and binding reagents in a fluid phase. Further, bead or particle solid supports can facilitate particular separation processes, such as separation of interaction identification moieties from analytes, and/or dissociation of binding reagents from analytes. In some configurations, a bead or particle solid support may comprise a magnetic bead, an electrically-charged bead, or a sedimentation bead. Some methods set forth herein do not require optical resolvability between analytes, so two adjacent analytes may be attached to a solid support at an optically non-resolvable distance from each other (e.g., less than about 500 nanometers (nm), 400 nm, 300 nm, 200 nm, 100 nm, or less than 50 nm).

A method may comprise a step of delivering a fluid comprising a binding reagent, or a plurality thereof, to an analyte, or a population thereof, in a fluid phase. A method may comprise a step of delivering a fluid comprising a binding reagent to a vessel, in which the vessel contains an analyte. Delivering a fluid comprising a binding reagent to an analyte may comprise delivering a first fluid phase comprising the binding reagent to a second fluid phase comprising an analyte. Delivering a fluid comprising a binding reagent to an analyte may further comprise mixing the first fluid phase comprising the binding reagent with the second fluid phase comprising the analyte. In some cases, a method may comprise a step of mixing the first fluid phase comprising the binding reagent with the second fluid phase comprising the analyte, thereby forming a single fluid phase comprising the binding reagent and the analyte. Alternatively, delivering a fluid comprising a binding reagent to an analyte may further comprise contacting the first fluid phase comprising the binding reagent with the second fluid phase comprising the analyte. For example, a method may comprise forming an emulsion of a first fluid phase in a second fluid phase, or vice versa.

In some cases, a method may involve delivering pluralities of binding reagents to one or more analytes in a serial or sequential fashion. A method may involve removing a first plurality of binding reagents from contact with one or more analytes before delivering a second plurality of binding reagents to the one or more analytes. For example, analytes attached to magnetic beads can be separated magnetically from a liquid phase, allowing removal and delivery of fluids to a vessel containing the analytes. In some cases, a method may involve delivering one or more analytes to a plurality of binding reagents in a serial or sequential fashion. A method may involve removing one or more analytes from contact with a first plurality of binding reagents before delivering the one or more analytes to a second plurality of binding reagents. For example, binding reagents attached to magnetic beads can be separated magnetically from a liquid phase, allowing removal and delivery of fluids to a vessel containing the binding reagents.

Alternatively, one or more binding reagents can be contacted to one or more analytes in a “one-pot” approach that allows for multiple instances of binding interactions to occur. In some cases, a method may include providing a pulsed, cyclical, or oscillatory binding condition that modulates the likelihood of binding interactions occurring. In some cases, temperature, pH, or ionic strength of a fluid in a vessel can be varied to facilitate binding interactions then inhibit binding interactions. For example, the temperature of a fluid in a vessel can be varied in a substantially sinusoidal manner (e.g., varying between 15 degrees Celsius and 30 degrees Celsius), thereby facilitating binding interactions at lower temperatures and dissociation at higher temperatures. In another example, the pH of a fluid in a vessel can be varied in a substantially sinusoidal manner (e.g., varying between pH 3.0 and pH 7.4), thereby facilitating binding interactions at higher pH and dissociation at lower pH. In particular cases, modulation of a fluidic property such as pH or ionic strength can include delivering a fluid that is substantially devoid of binding reagents and/or analytes to a vessel containing analytes and binding reagents.

A method set forth herein may be facilitated by providing to a fluidic medium a component that increases the proximity between analytes and binding reagents as well as other components utilized in a method set forth herein (e.g., ligation or polymerization enzymes) . . . . Secondary interactions, such as oligonucleotide hybridization, nucleic acid ligation or polymerization, or enzymatic labeling, may also be facilitated by the presence of a component that increases proximity. For example, binding reagents may be contacted to analytes in the presence of a crowding agent, such as a macromolecular crowding agent (e.g., polyethylene glycol, ficoll, dextran, bovine serum albumin, hemoglobin, polyvinylpyrrolidone, etc.), a small molecule crowding agent (e.g., sucrose, trehalose, sorbitol, glycerol, etc.), or a combination thereof. Accordingly, a method may include a step of providing a fluidic medium containing a crowding agent to a vessel.

A thermo-responsive polymer may be useful for increasing the proximity of analytes and affinity reagents as well as other components utilized in a method set forth herein (e.g., ligation or polymerization enzymes). Without wishing to be bound by theory, thermo-responsive polymers may be configured to form semi-solid structures above or below a critical temperature that encapsulate components such as affinity reagents, analytes, particles, and or enzymes, thereby increasing local concentrations of interacting components. A temperature change can induce dispersal of the polymer into the solution phase, thereby allowing diffusion of components away from each other. Alternatively, thermo-responsive polymers may act as temperature-sensitive crowding agents that reduce available volume for interacting components above or below the critical temperature.

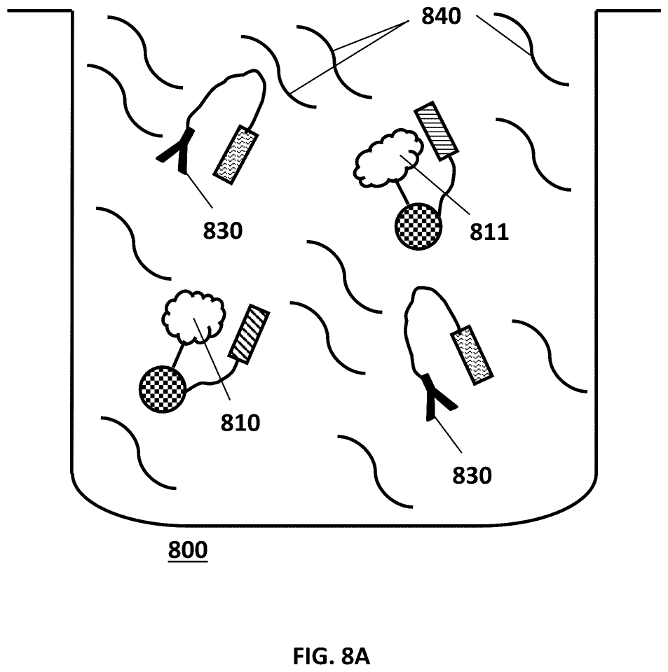

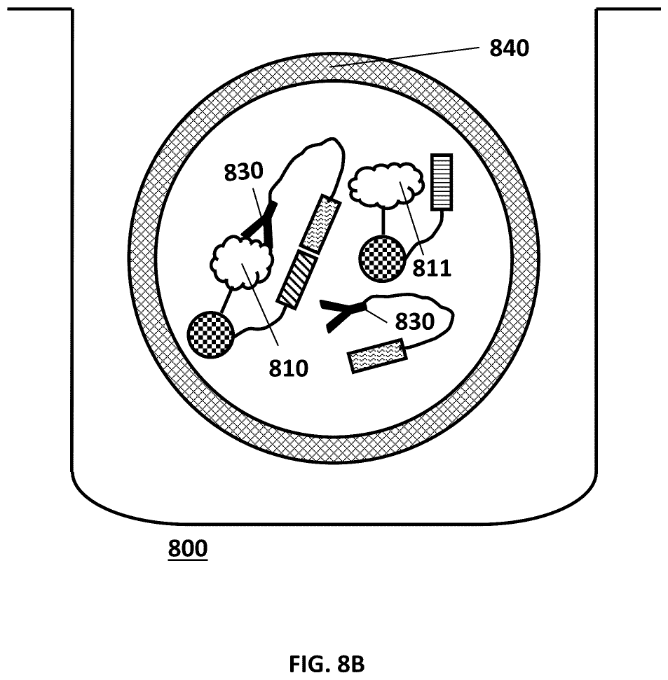

FIGS. 8A-8B illustrate a system containing a thermo-responsive polymer. FIG. 8A depicts a vessel 800 containing a fluidic medium, in which the fluidic medium contains a first analyte complex 810 and a second analyte complex 811. The analyte complexes each contain an associated analyte identification moiety and an analyte attached to a particle. The fluidic medium further contains a plurality of binding reagents 830 and a plurality of thermo-responsive polymer molecules 840. The system is at a first temperature, T₁, so the polymer molecules 840 are in a solvated or dispersed phase. FIG. 8B depicts the system at a temperature T₂, in which the temperature change has induced a phase change in the thermo-responsive polymer molecules 840. Accordingly, a condensed structure comprising the polymer molecules 840 has formed, encapsulating the analyte complexes 810 and 811, and the binding reagents 830. As shown, an affinity reagent 830 has formed a binding interaction with the analyte of analyte complex 810, thereby facilitating formation of an interaction identification moiety containing information from the analyte identification moiety of analyte complex 810 and information from the affinity reagent identification moiety of affinity reagent 830.

Thermo-responsive polymers that are soluble in aqueous media may be of particular interest to methods set forth herein. Exemplary thermo-responsive polymers can include poly(N-isopropylacrylamide), poly-2-(dimethylamino)ethyl methacrylate, hydroxypropyl cellulose, poly(vinylcapolactam), poly-2-isopropyl-2-oxazoline, polyvinyl methyl ester, polyethylene oxide, polyvinylmethylether, polyhydroxyethylmethacrylate, poly(N-acryloylglycinamide), ureido-functionalized polymers, N-vinylimidazole copolymers, 1-vinyl-2-(hydroxymethyl) imidazole, acrylamide/acrylonitrile copolymers, and combinations thereof.

A method may comprise a step of forming an interaction identification moiety. An interaction identification moiety may be formed after a binding reagent has bound to an analyte. An interaction identification moiety may be formed by bringing a first moiety (e.g., a moiety comprising a binding reagent identifier moiety) joined to the binding reagent in proximity to a second moiety (e.g., a moiety comprising an analyte identifier moiety) joined to an analyte. Preferably, an interaction between the first moiety and the second moiety that facilitates formation of the interaction identification moiety may be a weak or kinetically limited interaction that is only likely to occur if the binding reagent is bound to an analyte. An interaction identification may comprise a non-transitory moiety that remains detectable after a binding interaction between a binding reagent and an analyte has dissociated.

An interaction identification moiety may be formed by any suitable method. In some cases, an interaction identification moiety may be formed by attaching a first moiety to a second moiety. For example, an interaction identification moiety may be formed by ligating (e.g., nucleic acid ligation, peptide ligation) a first moiety comprising a binding reagent identifier moiety to a second moiety comprising an analyte identifier moiety. In some cases, an interaction identification moiety may be formed by copying or transferring information (e.g., a code or sequence) from a first moiety to a second moiety. For example, a nucleotide sequence of a binding reagent identifier nucleic acid strand may be transferred to an analyte identifier nucleic acid strand by polymerase extension, or vice versa. In some cases, an interaction identification moiety may be formed by altering an analyte identifier moiety. For example, an analyte identifier moiety comprising an Avi-tag may be biotinylated by a biotin ligase enzyme that is joined to a binding reagent.

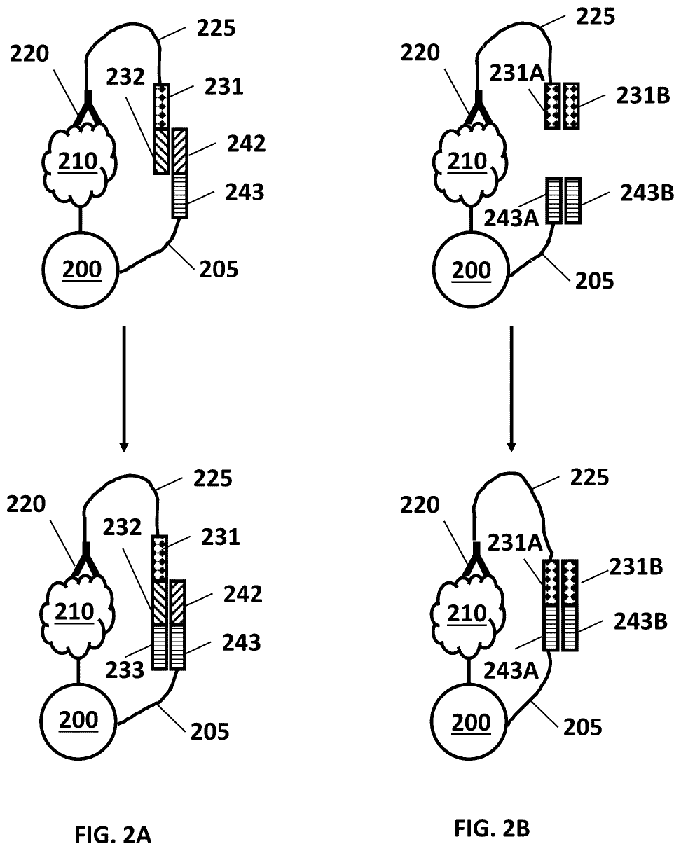

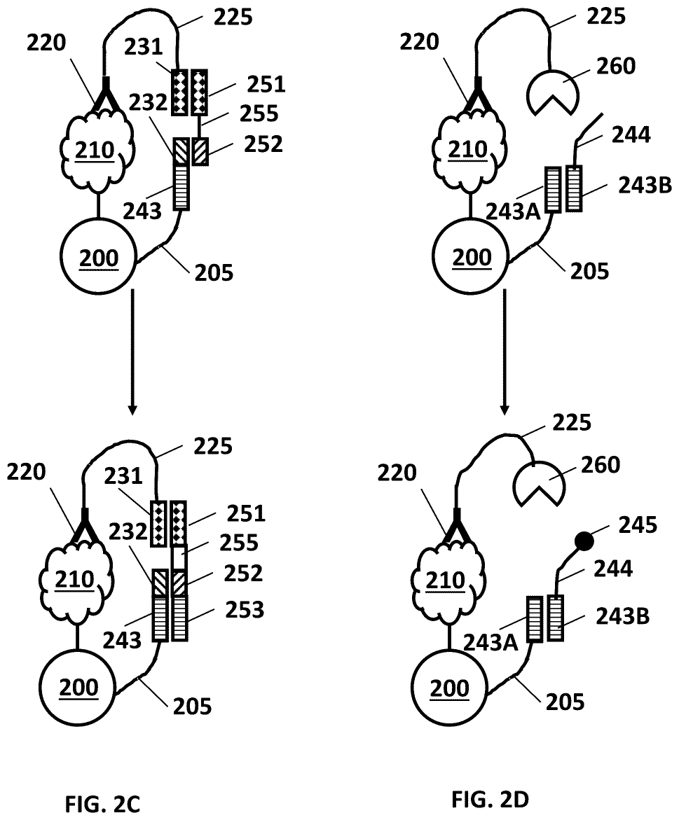

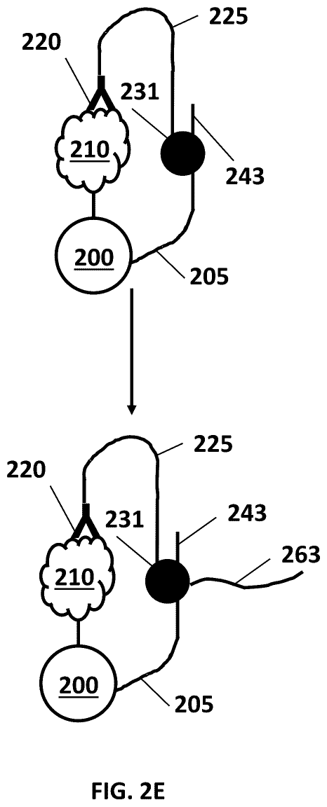

FIGS. 2A-2E depict various configurations of systems for recording a binding interaction that may be useful to incorporate into a system like the one depicted in FIG. 1A-1D. The interaction recording systems of FIGS. 2A-2E each contain an analyte 210 joined to or co-located with an analyte identifier moiety 243, optionally by attaching the analyte 210 and the analyte identifier moiety 243 to a particle 200. Alternatively, the analyte identifier moiety 243 may be attached to the analyte 210 without a particle 200. The analyte identifier moiety 243 may be attached to the particle 200 or analyte 210 by an optional linking moiety 205. Each of FIGS. 2A-2E illustrate a configuration in which a binding reagent comprising a binding reagent 220 is bound to the analyte 210.

FIG. 2A depicts a system for extending a nucleic acid strand to record a binding interaction. As shown in the upper configuration, the binding reagent is attached to an optional binding reagent identifier moiety 231 by an optional linking moiety 225. The binding reagent identifier moiety 231 further comprises a coupling nucleotide sequence 232, and the analyte identifier moiety 243 further comprises a complementary coupling nucleotide sequence 242, in which the coupling nucleotide sequence 232 and the complementary coupling nucleotide sequence 242 hybridize to couple the nucleic acid strands by nucleic acid hybridization. After the nucleic acid strands have hybridized (bottom configuration), extension of the nucleic acid strand containing the binding reagent identifier moiety 231 by a polymerase enzyme can incorporate the reverse complement 233 of the analyte-specific code of the analyte identifier moiety 243 into the nucleic acid strand (e.g., an analyte-specific code of ATGA will be incorporated into the extended nucleic acid strand as TACT). The binding interaction may be subsequently detected by sequencing the nucleic acid strand containing the reverse complement 233 of the analyte-specific code, thereby facilitating detection of the binding interaction between the analyte 210 and the binding reagent 220. It is readily recognized that the system can be reversed such that a binding reagent-specific code is incorporated on to the nucleic acid strand containing the analyte identifier moiety 243 by polymerase extension.

FIG. 2B depicts a system that forms a fusion molecule containing a binding reagent identifier moiety and an analyte identifier moiety. The upper configuration depicts the binding reagent 220 coupled to a double-stranded nucleic acid by an optional linking moiety 225. The double-stranded nucleic acid contains a nucleic acid strand containing the binding reagent identifier moiety 231A and a hybridized reverse complementary binding reagent identifier moiety 231B. The analyte 210 is joined with a double-stranded nucleic acid that comprises the analyte identifier moiety 243A and a hybridized reverse complementary analyte identifier moiety 243B. The lower configuration depicts formation of a fusion molecule comprising the reverse complementary binding reagent identifier moiety 231B attached to the reverse complementary analyte identifier moiety 243B. In the depicted configuration, the double-stranded nucleic acids may be joined by a double-stranded ligase. In other configurations, the binding reagent identifier moiety 231A and the analyte identifier moiety 243A could be ligated by a single-stranded ligase. In yet other configurations, the fusion molecule may be formed by a non-enzymatic coupling (e.g., a Click reaction). In some configurations, the binding reagent identifier moiety 231A and the analyte identifier moiety 243A may not contain nucleic acids (e.g., peptide moieties, polysaccharide moieties, etc.).

FIG. 2C depicts a system utilizing a bridging molecule. If a complex between a binding reagent 220 and an analyte 210 forms in the presence of the bridging molecule, the bridging molecule can bind to both the binding reagent and the analyte identifier moiety 243 that is joined with the analyte 210. As shown in the upper configuration, the binding reagent comprises a first nucleic acid strand, optionally comprising a binding reagent identifier moiety 231. The analyte is joined with a second nucleic acid strand containing an analyte identifier moiety 243 and an optional coupling moiety 232. The bridging molecule is configured to hybridize to the first nucleic acid strand and the second nucleic acid strand by a first nucleotide sequence 251 that is complementary to the first nucleic acid strand, and a second nucleotide sequence 252 that is complementary to the second nucleic acid strand. Optionally, the first nucleotide sequence 251 and the second nucleotide sequence 252 are attached by a linking moiety 255. In the lower configuration, the bridging molecule has been extended by a polymerase extension reaction, thereby incorporating the reverse complement of the analyte-specific code of the analyte identifier moiety 243 into the bridging molecule. Subsequently, the bridging molecule may be sequenced to determine if the binding interaction occurred, as shown by the presence of the reverse complement of the analyte-specific code.

FIG. 2D depicts a system that utilizes a modifying agent 260 as a binding reagent identifier moiety. The modifying agent 260 (e.g., a kinase, a biotin ligase, a sortase, etc.) is attached to the binding reagent 220 by an optional linking moiety 225. As shown in the lower configuration, when the modifying agent 260 is brought in proximity to a modifiable tag 244 that is attached to an analyte identifier moiety 243B by the binding of the binding reagent 220 to the analyte 210, the modifiable tag 244 may be altered to contain a detectable label 245. In the depicted configuration, the analyte identifier moiety 243B attached to the modifiable tag 244 is dissociable from a complementary handle 243A, so that the analyte identifier moiety 243B can be recovered and detected subsequently. In some cases, the detectable label 245 may be a ligand or reactive group (e.g., biotin, a carboxyl, an amine, etc.) that facilitates capture of the analyte identifier moiety 243B after a binding interaction. For example, a biotinylated modifiable tag 244 (e.g., an avi-tag) may be captured by a solid support containing streptavidin molecules. If the modifiable tag 244 is not biotinylated (due to the absence of a binding interaction), the moiety will not be retained on the streptavidin-coated solid support.

FIG. 2E depicts a system that is configured to synthesize an interaction identification moiety when the analyte 210 is bound by the binding reagent 220. The binding reagent 220 is attached to a binding reagent identifier moiety 231 comprising a ribosome. The analyte 210 is joined with an analyte identifier moiety 243 comprising a ribonucleic acid strand. The ribonucleic acid strand can contain an analyte-specific code containing a codon sequence. When the binding reagent identifier moiety 231 is in proximity to the analyte identifier moiety 243, the ribosome may bind to the ribonucleic acid strand, initiating synthesis of a peptide strand 263 from the ribosome in the presence of amino acid substrates. The peptide strand may be subsequently sequenced to identify the analyte-specific code, thereby detecting the binding interaction between the analyte 210 and the binding reagent 220. Additional methods, systems, and compositions for forming

Methods set forth herein can include forming an interaction identification moiety. The interaction identification moiety can be any conceivable moiety that i) is formed due to a binding interaction between an analyte and a binding reagent; ii) is detectable after dissociation of the binding interaction between the analyte and the binding reagent, and iii) contains an analyte-specific code that is unique to the analyte involved in the binding interaction. In some configurations, an interaction identification moiety may further comprise a binding reagent-specific code, a vessel-specific code, and/or an assay sequence-specific code. Detection of the binding reagent-specific code may facilitate identification of the binding reagent that bound to the analyte. Detection of a sequence-specific code may facilitate identification of the step of a sequential or serial assay at which the binding reagent was bound to the analyte. Detection of a vessel-specific code may facilitate identification of the vessel in which the binding reagent was bound to the analyte. It may be useful to form an interaction identification moiety including a binding reagent-specific code and/or an assay-sequence specific code if two or more binding reagents are multiplexed (e.g., simultaneously contacted to an analyte at the same step) or if pools of interaction identification moieties formed by different binding reagents (multiplexed or in differing steps) are detected simultaneously.

An interaction identification moiety may comprise a polymer strand containing a sequence of polymerized residues. Preferably, the polymer strand is readily sequenced by a sequencing method or device (e.g., DNA sequencing, RNA sequencing, peptide sequencing, polysaccharide sequencing). Accordingly, the polymer strand of an interaction identification moiety may be sequenced by a sequencing device, thereby detecting one or more of: i) an analyte-specific code, ii) a binding reagent-specific code, iii) an assay sequence-specific code, and iv) a vessel-specific code. Each of the analyte-specific code, the binding reagent-specific code, the vessels-specific code, and the assay sequence-specific code may comprise residue sequences that are determinable by a sequencing device. Depending upon the configuration of a method of forming an interaction identification moiety, the analyte-specific code, the binding reagent-specific code, the vessel-specific code, and/or the assay sequence-specific code may be contiguous (i.e., not separated by any intervening residues, pluralities of residues, or other linking moieties) in the polymer strand of the interaction identification moiety. Alternatively, the analyte-specific code, the binding reagent-specific code, the vessel-specific code, and/or the assay sequence-specific code may be non-contiguous (i.e., separated by any intervening residue, a plurality of residues, or other linking moiety) in the polymer strand of the interaction identification moiety.

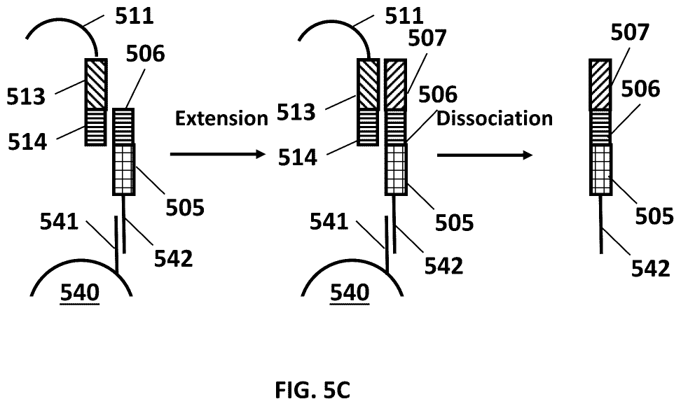

In some cases, forming an interaction identification moiety can comprise forming a nucleic acid strand. Forming a nucleic acid strand can comprise hybridizing a first nucleic acid strand of a binding reagent identifier moiety (e.g., a nucleic acid strand comprising a binding reagent-specific nucleotide sequence) to a second nucleic acid strand of an analyte identifier moiety (e.g., a nucleic acid strand comprising an analyte-specific code). After hybridizing the first nucleic acid strand of the binding reagent identifier moiety to the second nucleic acid strand of the analyte identifier moiety, a method may further comprise extending the first nucleic acid strand or the second nucleic acid strand by a polymerase extension reaction, thereby forming the interaction identification moiety. If the first nucleic acid strand is extended by a polymerase extension reaction, the analyte-specific code may be transferred to the first nucleic acid strand. The skilled person will readily recognize that the analyte-specific code would be added to the first nucleic acid strand as its reverse complementary sequence. Likewise, if the second nucleic acid strand is extended by a polymerase extension reaction, the binding reagent-specific code and/or assay sequence-specific code and/or vessel-specific code may be transferred to the second nucleic acid strand. Methods of forming interaction identification moieties by polymerase extension are shown in FIGS. 2A, 2C, 5B, and 5C.

Alternatively, forming an interaction identification moiety comprising a nucleic acid strand may comprise ligating a first nucleic acid strand of a binding reagent identifier moiety to a second nucleic acid strand of an analyte identifier moiety. In some cases, ligating the first nucleic acid strand of the binding reagent identifier moiety to the second nucleic acid strand of the analyte identifier moiety can comprise performing a single-stranded ligation reaction. After performing a single-stranded nucleic acid ligation reaction, the binding reagent and analyte may be effectively formed into a non-dissociable complex by the nucleic acid ligation product. Accordingly, forming an interaction identification moiety may further comprise copying the nucleic acid ligation product by a polymerase chain reaction. Performing the polymerase chain reaction may require providing a priming oligonucleotide and a polymerase enzyme. After performing the polymerase chain reaction, the reverse complement of the ligation product may be readily dissociable by nucleic acid dehybridization, thereby providing an interaction identification product. In other cases, ligating the first nucleic acid strand of the binding reagent identifier moiety to the second nucleic acid strand of the analyte identifier moiety can comprise performing a double-stranded ligation reaction, thereby forming the interaction identification moiety. Formation of an interaction identification moiety by double stranded ligation is shown in FIG. 2B. After the double stranded ligation reaction, the interaction identification moiety may be readily de-hybridized from the binding reagent-analyte complex.

In some cases, forming an interaction identification moiety can comprise forming a peptide strand. In some cases, an analyte identifier moiety can comprise a peptide strand or tag. In some cases, an analyte identifier moiety can comprise a nucleic acid strand attached to a peptide strand (e.g., an affinity tag). In such cases, a binding reagent identifier moiety may comprise an enzyme, wherein the enzyme attaches a binding reagent identifier tag to the peptide strand, thereby forming the interaction identification moiety. An enzyme may chemically modify a peptide strand to provide a reactive functional group (e.g., carboxylation by a carboxylase; amination by a transaminase or an amine dehydrogenase). In other cases, an enzyme may chemically modify a peptide strand to attach a binding ligand (e.g., biotinylation by biotin ligase, attachment of a moiety by a sortase, etc.).

Polymer strands containing multiple differentiable residues may be particularly useful for forming interaction identification moieties due to the large quantity of unique codes that can be composed from a relatively small quantity of residues. For example, the quantity of unique codes that can be composed for nucleic acids goes as 4^{N}, where N is the quantity of residues in a nucleic acid sequence, so 5 residues could produce over 1000 unique codes, and 10 residues could produce over 1000000 unique codes. Likewise, the quantity of unique codes that can be composed for peptides goes as 20^{N}, where N is the quantity of residues in a peptide sequence, so 3 residues could produce 8000 unique codes, and 5 residues could produce over 3000000 unique codes. Accordingly, the length of a code incorporated into an identifier moiety (e.g., an analyte-specific code, a binding reagent-specific code, a vessel-specific code, an assay sequence-specific code) may be chosen based upon the number of entities that need to be distinguished. An identifier moiety (e.g., an analyte-specific code, a binding reagent-specific code, a vessel-specific code, an assay sequence-specific code) comprising a polymer strand may comprise at least about 1, 2, 3, 4, 5, 6, 7, 8, 9, 10, 11, 12, 13, 14, 15, 16, 17, 18, 19, 20, 25, 30, 40, 50 or more than 50 residues. Alternatively or additionally, an identifier moiety comprising a polymer strand may comprise no more than about 50, 40, 30, 25, 20, 19, 18, 17, 16, 15, 14, 13, 12, 11, 10, 9, 8, 7, 6, 5, 4, 3, 2, or 1 residues.

An interaction identification moiety comprising a polymer strand may include at least one coded residue sequence (e.g., an analyte-specific code) and optionally one or more additional coded residue sequences. An interaction identification moiety can further comprise one or more non-coded residues or residue sequences. For example, configurations of interaction identification moieties depicted in FIGS. 2A, 2B, 5B, and 5C can include residue sequences that facilitate hybridization of two nucleic acid strands, but do not necessarily contain coded information. An interaction identification moiety comprising a polymer strand may comprise at least about 1, 2, 3, 4, 5, 10, 15, 20, 25, 30, 40, 50, 60, 70, 80, 90, 100, or more than 100 residues. Alternatively or additionally, an interaction identification moiety comprising a polymer strand may comprise no more than about 100, 90, 80, 70, 60, 50, 40, 30, 25, 20, 15, 10, 5, 4, 3, 2, or 1 residues.

In some cases, an interaction identification moiety may record a single binding event between an analyte and an identifier moiety. Accordingly, an interaction identification moiety may contain a single analyte identification moiety and a single affinity reagent identification moiety. In other cases, an interaction identification moiety can include information from two or more binding events. Accordingly, an interaction identification moiety may contain a single analyte identification moiety and two or more affinity reagent identification moieties, or may contain a single affinity reagent identification moiety and two or more analyte identification moieties.

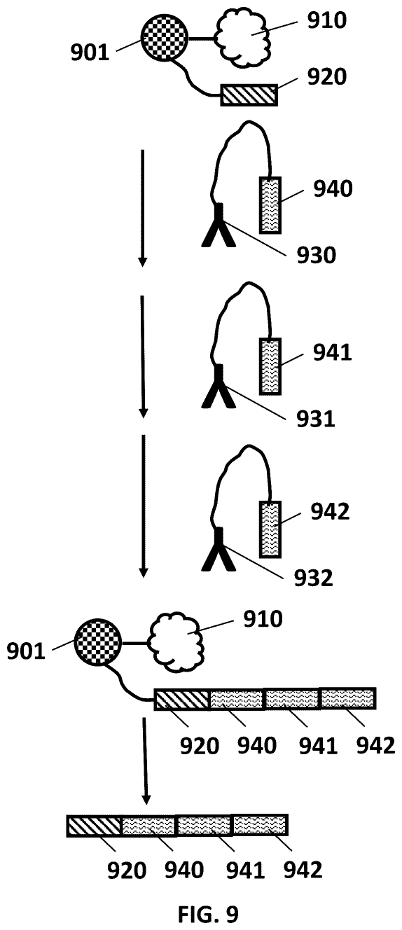

FIG. 9 illustrates a method including serial or multiplexed detection, in which an analyte 910 forms binding interactions with affinity reagents 930, 931, and 932. The analyte 910 is attached to a solid support or particle 901 and an analyte identification moiety 920. The affinity reagents 930, 931, and 932 are attached to affinity reagent identification moieties 940, 941, and 942, respectively. Each detected binding event leads to the transfer of information from the affinity reagent identification moieties 940, 941, and 942 to an interaction identification moiety containing the analyte identification moiety 920. Subsequently, the interaction identification moiety containing the analyte identification moiety 920 and the affinity reagent identification moieties 940, 941, and 942 can be dissociated from the solid support or particle 901. In some configurations, the system can be reversed, such that each affinity reagent is attached to an interaction identification moiety that contains information from the analyte identification moiety 920 plus the respective affinity reagent identification moiety.

Methods for extending an interaction identification moiety to include information from multiple detection events are known in the art. Oligonucleotide interaction identification moieties can be extended by ligation or PCR-based methods. If a PCR-based method is used, each detection event may include transferring a primer sequence that facilitates binding of an identification moiety during a subsequent detection event. Useful methods, systems, and compositions for recording multiple nucleotide barcodes in a single oligonucleotide are provided in US Patent Publication Nos. 2019/0145982 A1; 2020/0348308 A1; or 2020/0348307 A1, each of which is incorporated herein by reference in its entirety.

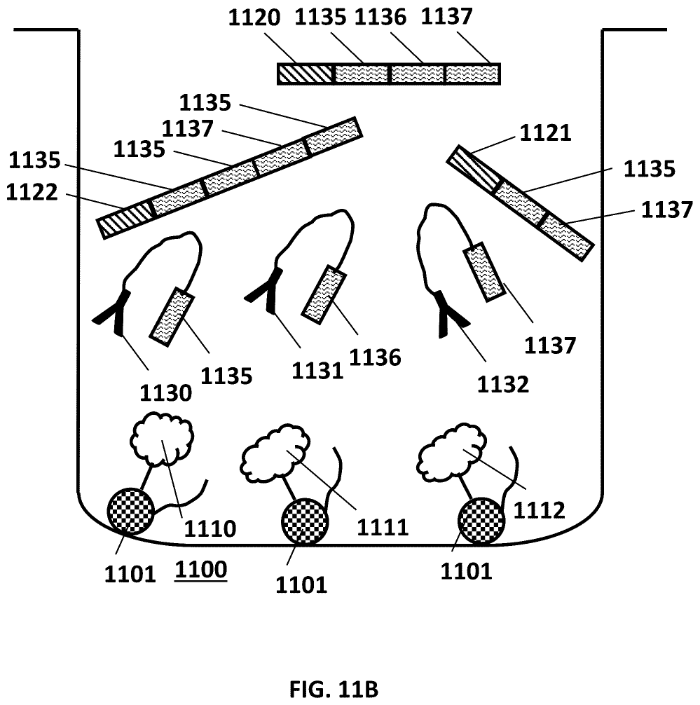

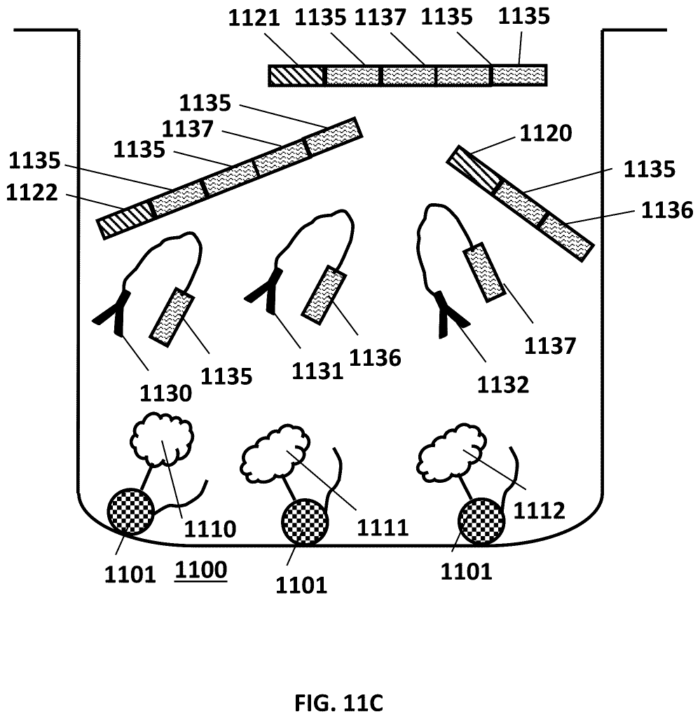

Formation of interaction identification moieties containing information from multiple binding events may be useful for “one-pot” methods, in which binding reagents and analytes can form multiple binding interactions before binding reagents are separated from the analytes. FIGS. 11A-11C depict aspects of a “one-pot” system that utilizes multi-interaction interaction identification moieties. FIG. 11A depicts a vessel 1100 containing unknown analytes 1110, 1111, and 1112. Each analyte is attached to a particle or solid support 1101, and analytes 1110, 1111, and 1112 are attached to analyte identification moieties 1120, 1121, and 1122, respectively. The vessel further comprises affinity reagents 1130, 1131, and 1132, which are attached to affinity reagent identification moieties 1135, 1136, and 1137, respectively. Affinity reagent 1135 differs from affinity reagents 1136 and 1137 with respect to binding specificity, and affinity reagent 1136 differs from affinity reagent 1137 with respect to binding specificity. FIG. 11B depicts the vessel 1100 after the affinity reagents and analytes have undergone multiple binding interactions. Interaction identification moieties have been dissociated from particles 1101. The leftmost interaction identification moiety contains analyte identification moiety 1122, a single copy of affinity reagent identification moiety 1137, and three copies of affinity reagent identification moiety 1135. The rightmost interaction identification moiety contains analyte identification moiety 1121, a single copy of affinity reagent identification moiety 1137, and a single copy of affinity reagent identification moiety 1135. The center interaction identification moiety contains a single copy of each affinity reagent identification moiety (1135, 1136, 1137). It may be concluded that analyte 1120 differs from analytes 1121 and 1122 due to the difference in binding profiles. It is also possible that analyte 1121 differs from 1122 due to the decreased likelihood of analyte 1121 interacting with affinity reagent 1130.

FIG. 11C depicts a differing outcome from the system of FIG. 11B. The leftmost and center interactions moieties each comprise a single copy of affinity reagent identification moiety 1137 and three copies of affinity reagent identification moiety 1135, albeit in a different ordering of interactions. The rightmost interaction identification moiety contains a single copy of analyte identification moiety 1120, a single copy of affinity reagent identification moiety 1135, and a single copy of affinity reagent identification moiety 1136. It may be concluded that analytes 1121 and 1122 can be the same species of analyte, while analyte 1120 differs from analytes 1121 and 1122.

A method of the present disclosure can include recording interactions between binding reagents and analytes under two differing conditions. Differing conditions can include differing structural states of the analytes (e.g., folded vs. unfolded proteins or other analytes) as well as environmental conditions surrounding the analytes and binding reagents (e.g., differing fluid compositions, differing temperatures, differing fluid pH, differing fluid ionic strength, etc.). A method may include the steps of: (i) contacting a first plurality of binding reagents to one or more analytes in the presence of a first binding condition; (ii) forming one or more interaction identification moieties in the presence of the first binding condition; (iii) contacting a second plurality of binding reagents to one or more analytes in the presence of a second binding condition, in which the first plurality of binding reagents is substantially identical to the second plurality of binding reagents; and (iv) forming one or more interaction identification moieties in the presence of the second binding condition.

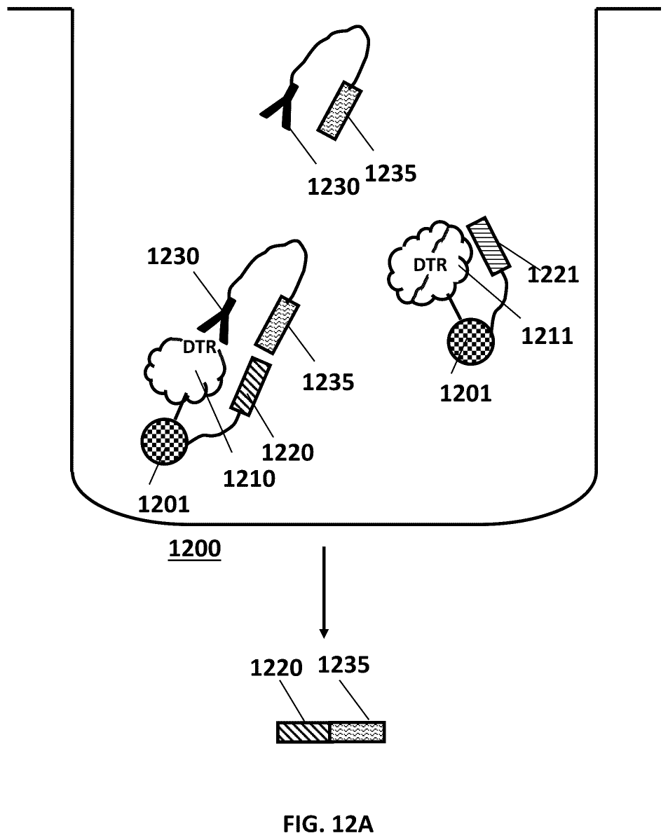

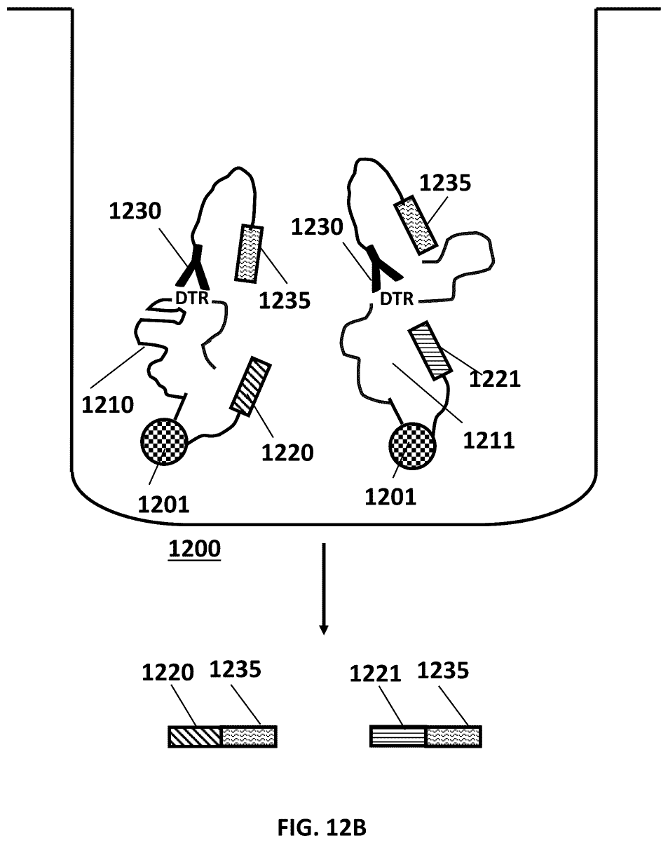

FIGS. 12A-12B illustrate a method containing steps of contacting binding reagents with analytes. FIG. 12A depicts a first configuration, in which a vessel contains a plurality of affinity reagents 1230 and analytes 1210 and 1211. Each analyte is attached to a particle 1201, and analytes 1210 and 1211 are associated with analyte identification moieties 1220 and 1221, respectively. Each affinity reagent 1230 is attached to an affinity reagent identification moiety 1235. The affinity reagent has a binding specificity for an amino acid epitope having the amino acid sequence DTR. The epitope DTR is accessible near the surface of analyte 1210 and buried internally in analyte 1211. Accordingly, an affinity reagent is able to bind to the surface-exposed epitope of analyte 1210 but cannot bind to the occluded epitope of analyte 1211. Subsequently, an interaction identification moiety is obtained containing information from the analyte identification moiety 1220 and the affinity reagent identification moiety 1235. FIG. 12B depicts a second configuration in which a binding condition has been provided that causes full or partial denaturation of the analytes 1210 and 1211. Accordingly, the epitope DTR is accessible to affinity reagents 1230 for both analytes. Subsequently, interaction identification moieties are obtained containing information from the analyte identification moieties 1220 and 1221 and the affinity reagent identification moiety 1235.

Differing binding conditions can be formed by altering a fluid in a vessel (e.g., adding a second fluid or other chemical component to the vessel, heating a fluid in the vessel, etc.) or by replacing a first fluid in the vessel with a second fluid. A binding profile generated during a method of analyte characterization may include binding information between an analyte and an affinity reagent under two or more binding conditions. Methods of characterizing analytes by utilizing differing binding conditions are further described in U.S. patent application Ser. No. 19/226,698, which is herein incorporated by reference in its entirety.

Methods and systems set forth herein may be particularly useful for detecting binding interactions between a population of analytes and a plurality of binding reagents at binding equilibrium. The binding equilibrium that forms between a population of analytes and a plurality of binding reagents may be determined in part by the respective concentrations of analytes and binding reagents, as well as the binding specificities of the binding reagents. A binding specificity of a binding reagent may be characterized as comprising one or more superordinate binding specificities, and/or one or more subordinate binding specificities. Superordinate and subordinate binding specificities may also be referred to as on-target and off-target binding, respectively. A superordinate binding specificity may refer to a molecule, a set of molecules, an epitope, or a set of epitopes to which a binding reagent has a strongest binding affinity. A superordinate binding specificity, as characterized by a dissociation constant, K_{D}, may refer to a binding interaction of the binding reagent with a binding target that has a K_{D} of no more than about 100 nanoMolar (nM), 50 nM, 40 nM, 30 nM, 20 nM, 10 nM, 5 nM, 1 nM, 0.5 nM, 0.1 nM, or less than 0.1 nM. Likewise, a subordinate binding specificity may refer to a molecule, a set of molecules, an epitope, or a set of epitopes to which a binding reagent has a weaker binding affinity than a superordinate binding specificity. A subordinate binding specificity, as characterized by a dissociation constant, K_{D}, may refer to a binding interaction of the binding reagent with a binding target that has a K_{D} of at least about 50 nanoMolar (nM), 100 nM, 200 nM, 500 nM, 1 microMolar (μM), or more than 1 μM.

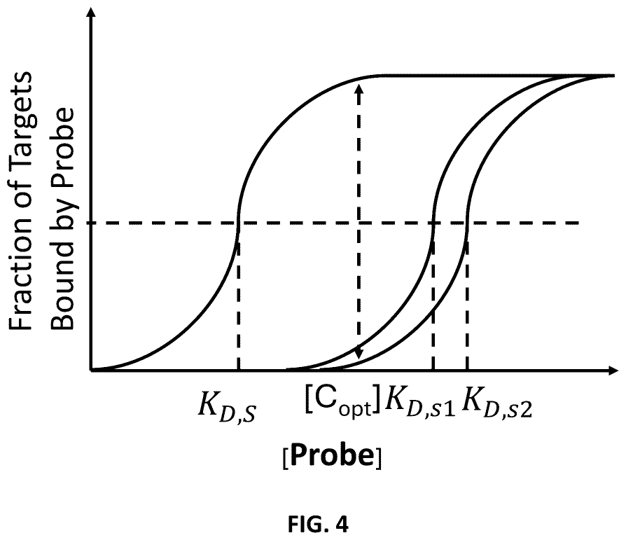

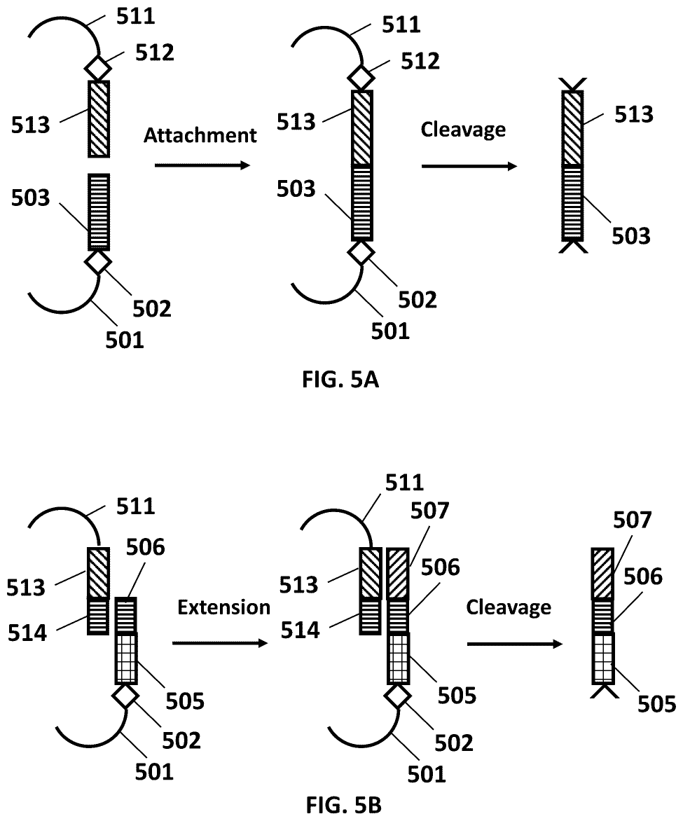

If the superordinate and subordinate binding interactions of a binding reagent have been characterized, it may be possible to determine a concentration at which the difference between the superordinate and subordinate binding reagent interactions are maximized. FIG. 4 illustrates fraction of binding targets bound as a function of binding reagent concentration. The left curve represents the binding equilibrium for the binding reagent and its superordinate binding target. The two right curves represent the binding equilibria for the binding reagent and two of its subordinate binding targets. The respective dissociation constants (superordinate—K_{D,S}; subordinates—K_{D,s1} and K_{D,s2}) are shown as the binding reagent concentrations at which the interactions achieve 50% binding of their respective targets. FIG. 4 also includes an optimal concentration, Copt. At the optimal concentration, it can be observed that the fraction of bound superordinate binding targets at equilibrium approaches 100% (e.g., at least about 80%, 90%, 95%, 99%, etc.), while the fraction of bound subordinate binding targets at equilibrium is relatively small (e.g., no more than about 10%, 5%, 1%, 0.5%, 0.1% etc.). In some cases, the optimal concentration may be calculated as the arithmetic or geometric mean of the dissociation constants of all characterized binding interactions (i.e., superordinate and subordinate binding interactions).

After forming an interaction identification moiety, a method may further comprise a step of separating the interaction identification moiety from a binding reagent, an analyte, or a complex comprising the binding reagent and analyte. A method of separating an interaction identification moiety can depend in part on the composition of the interaction identification moiety (e.g., nucleic acid strand, peptide strand, polysaccharide strand) and/or the type of attachment of the interaction identification moiety to an associated binding reagent, analyte, or complex thereof. In some cases, separating the interaction identification moiety from the binding reagent or the analyte comprises de-hybridizing the interaction identification moiety, enzymatically cleaving the interaction identification moiety, chemically cleaving the interaction identification moiety, or photolytically cleaving the interaction identification moiety. Accordingly, a method may comprise a step of providing: i) heat to an interaction identification moiety; ii) a denaturing agent to an interaction identification moiety, iii) an enzyme (e.g., a restriction enzyme, a protease) to an interaction identification moiety, iv) a chemical agent (e.g., an acidic species, a basic species, a nucleophile, an electrophile, etc.) to an interaction identification moiety; or v) a photon of light to an interaction identification moiety.

Separating an interaction identification moiety from a binding reagent, an analyte, or a complex thereof may further comprise segregating a solid support to which the binding reagent, analyte, or complex thereof are attached within a fluid phase containing the interaction identification moiety. Segregating a solid support may comprise transferring the solid support to the surface of a vessel containing the interaction identification moiety. A solid support comprising a magnetic bead, an electrically-charged bead, or a sedimentation bead may be especially useful. In some cases, transferring a solid support to a surface of a vessel may comprise: i) providing a magnetic field or an electric field in a vessel comprising a fluid phase containing an interaction identification moiety and a solid support comprising a magnetic bead or an electrically-charged bead; and ii) transferring the solid support to a surface of the vessel in the presence of the magnetic field or electric field. In other cases, transferring a solid support to a surface of a vessel may comprise: i) providing a sedimentation force (e.g., a centripetal force by centrifugation; passive gravitational settling) in a vessel comprising a fluid phase containing an interaction identification moiety and a solid support comprising a sedimentation bead; and ii) transferring the solid support to a surface of the vessel in the presence of the sedimentation force. After segregating a solid support from an interaction identification moiety within a fluid phase, a method may further comprise withdrawing from a vessel a portion of the fluid phase containing the interaction identification moiety.

After an interaction identification moiety has been formed, the interaction identification moiety may be attached to an analyte, binding reagent, particle, or solid support. Accordingly, before a step of detecting the interaction identification moiety, the interaction identification moiety may be dissociated from the analyte, binding reagent, particle, or solid support. In some configurations, an interaction identification moiety that is associated to an analyte, binding reagent, particle, or solid support may be associated by the attachment of a nucleic acid strand of the interaction identification moiety with a nucleic acid strand of the analyte, binding reagent, particle, or solid support. In such configurations, the interaction identification moiety can be dissociated from the analyte, binding reagent, particle, or solid support by any suitable method of de-hybridizing nucleic acids, including heat or use of a denaturant agent (e.g., formamide, guanidine, sodium salicylate, dimethyl sulfoxide, propylene glycol, urea, sodium hydroxide, etc.). FIG. 5C depicts the formation and dissociation of an interaction identification moiety that is attached by nucleic acid hybridization. In an initial configuration (left), an analyte identifier moiety comprising an analyte-specific code 505 and a coupling nucleotide sequence 506 is attached to a particle 540 by nucleic acid hybridization between a first nucleic acid strand 542 of the analyte identifier moiety and a second nucleic acid strand 541 attached to the particle 540. The analyte identifier moiety is attached to a binding reagent identifier moiety that is attached to a binding reagent (not shown) by a linking moiety 511. The binding reagent identifier moiety comprises a binding reagent-specific code 513 and a complementary coupling nucleotide sequence 514 that is hybridized to the coupling nucleotide sequence 506. As shown in the middle configuration, an interaction identification moiety is formed by polymerase extension of the analyte identifier moiety, thereby attaching the reverse complement 507 of the binding reagent-specific code 513 to the coupling nucleotide sequence 506. The right configuration depicts dissociation of the interaction identification moiety by dehybridization of the first nucleic acid strand 542 from the second nucleic acid strand 541. This configuration may be convenient because a new analyte identifier moiety can be attached to the second nucleic acid strand to

In other configurations, an interaction identification moiety that is associated with an analyte, binding reagent, particle, or solid support may be associated with one or more non-dissociable attachments. A non-dissociable attachment may include a covalent attachment or an irreversible receptor-ligand binding pair (e.g., streptavidin-biotin). FIGS. 5A-5B depict useful configurations for dissociating an interaction identification moiety that is attached to an analyte, binding reagent, particle, or solid support by a non-dissociable attachment. FIG. 5A depicts the formation of an interaction identification moiety by attachment of a first moiety 503 to a second moiety 513. The attachment of the first moiety 503 to the second moiety 513 may occur by ligation (e.g., enzyme-mediated nucleic acid ligation, native peptide ligation, Staudinger peptide ligation, Ser/Thr peptide ligation, etc.) or any other suitable form of attachment (e.g., Click-type reaction between terminal functional groups). The first moiety 503 is attached to an optional linking moiety 501 that contains a first cleavable moiety 502. The second moiety 513 is attached to an optional linking moiety 511 that contains a second cleavable moiety 512. After the interaction identification moiety has been formed by attachment of the first moiety 503 to the second moiety 513 (middle configuration), the first cleavable moiety 502 and the second cleavable moiety 512 may be cleaved, thereby producing a dissociated interaction identification moiety (right configuration). Numerous cleavable moieties can be utilized in a system as depicted in FIG. 5A, such as photocleavable or photolabile groups (cleaved by a photon of light), single- or double-stranded nucleic acids (cleaved by a restriction enzyme), or peptides (cleaved by a protease).

FIG. 5B depicts a similar configuration to that of FIG. 5A, but with an interaction identification moiety formed by polymerase extension of a first moiety. In this configuration, it may only be necessary to incorporate a cleavable moiety 502 into one of the moieties. As shown, a first moiety comprising a first code sequence 505 (e.g., an analyte-specific code, a binding reagent-specific code, a vessel-specific code, an assay sequence-specific code) is attached to a second moiety comprising a second code sequence 513 by hybridization of a coupling nucleotide sequence 506 of the first moiety to a complementary coupling nucleotide sequence 514 of the second moiety. The first moiety is attached to an optional linking moiety 501 that contains the cleavable moiety 502. The second moiety is attached to an optional linking moiety 511. After polymerase extension (middle configuration), the reverse complement 507 of the second code sequence 513 is attached to the coupling nucleotide sequence 506 of the first moiety. After polymerase extension, the first moiety and the second moiety may be dehybridized, and the cleavable moiety 502 may be cleaved by a method set forth herein, thereby producing a dissociated interaction identification moiety (right configuration).

In some configurations, it may be preferable to enrich interaction identification moieties from a population of containing moieties other than interaction identification moieties. For example, when interaction identification moieties are formed by attaching an analyte-specific code to a binding reagent-specific code, it may be preferable to deplete moieties containing only an analyte-specific code. Such moieties would indicate no detected binding interaction between a binding reagent and the analyte associated with the analyte-specific code. In another example, when interaction identification moieties are formed by attaching a detectable label to an analyte-specific code, it may be preferable to deplete moieties containing only an analyte-specific code without the detectable label. Such moieties would indicate no detected binding interaction between a binding reagent and the analyte associated with the analyte-specific code.

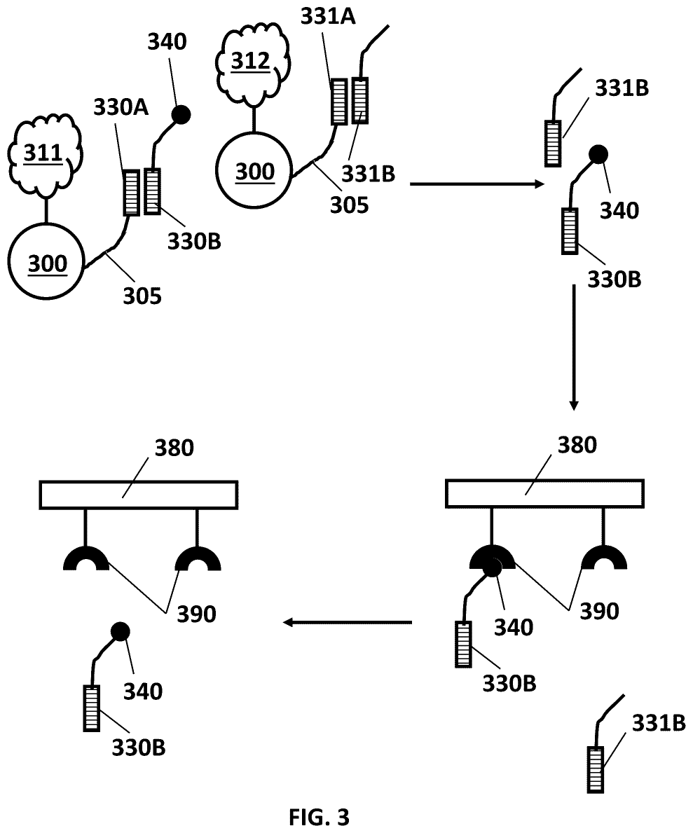

FIG. 3 illustrates a method of enriching interaction identification moieties from a population of polymer strands. The depicted method may be useful after forming interaction identification moieties by a method such as that depicted in FIG. 2D. In the upper left configuration, a first analyte 311 is joined to a first analyte identifier moiety 330B, and a second analyte 312 is joined to a second analyte identifier moiety 331B. The respective analyte identifier moieties 330B and 331B are attached to dissociable coupling moieties 330A and 331B (e.g., by nucleic acid hybridization). The dissociable coupling moieties are attached to linking moieties 305 that are attached to a particle 300 that joins each analyte (311, 312) with its respective dissociable coupling moiety. The analyte identifier moiety 330B joined to analyte 311 comprises a modifiable tag that has been attached to a detectable label 340, thereby forming an interaction identification moiety. The modifiable tag of analyte identifier moiety 331B has not been modified. In the upper right configuration, the analyte identifier moieties 330B and 331B have been dissociated from their respective dissociable coupling moieties 330A and 331A. In the lower right configuration, the analyte identifier moieties 330B and 331B are contacted to a solid support 380 comprising a plurality of attachment sites 390 that are configured to bind to the detectable label 340. Accordingly, the interaction identification moiety containing the analyte identifier moiety 330B is bound to the solid support 380. The analyte identifier moiety 331B is not bound to the solid support 380, and may be rinsed away after the interaction identification moiety is bound. In the bottom left configuration, the interaction identification moiety is dissociated from the solid support 380, thereby providing a fraction enriched for interaction identification moieties and depleted of polymer strands not containing the detectable label 340. Table I lists some possible configurations for forming interaction identification moieties that may facilitate enrichment. Table I includes information on the types of moieties that may be added or modified, thereby forming an interaction identification moiety, methods of adding them to analyte identifier moieties to form an interaction identification moiety, and the type of purification group that may facilitate enrichment of formed interaction identification moieties. Additional information on useful purification or affinity tags can be found in Kemple, M. E., et al. “Overview of Affinity Tags for Protein Purification.” Curr Protoc Protein Sci. (2013), which is herein incorporated by reference in its entirety.

TABLE I

| | Type of Moiety to be | Method of Addition or | Purification Group |
|---|---|---|---|
| Method | Added or Modified | Modification | on Solid Support |
| Biotin Ligase | Peptide (Avi-tag) | Enzymatic biotinylation of | Streptavidin, Avidin |
| | | Avi-tag peptide | |
| Capture Oligos | Oligonucleotide | Addition of capture oligo | Complementary |
| | | nucleotide sequence by | oligonucleotide |
| | | ligation, extension, or | |
| | | covalent attachment of | |
| | | oligonucleotide | |
| Purification or | Peptide | Enzymatic or covalent | Peptide-specific |
| Affinity Tags | | attachment of peptide | binding reagent or |
| | | purification tag sequence | binding protein |
| | | (e.g., His-tag, B-tag, FLAG- | |
| | | tag, etc) | |
| Reactive | Oligonucleotide, | Enzymatic or chemical | Complementary |
| Groups | peptide, | modification of oligo or | reactive groups |
| | polysaccharide, | peptide (e.g., carboxylation, | |
| | synthetic polymer | amination, etc.); | |
| | | incorporation or attachment | |
| | | of modified nucleotides or | |
| | | non-natural amino acids | |
| Terminal | Oligonucleotide | Addition of poly-T | Poly-A |
| nucleotidyl | | nucleotide sequence on | oligonucleotide |
| transferase | | oligonucleotide terminus; | |
| | | extension length can indicate | |
| | | length of time affinity | |
| | | reagent attached to | |
| | | transferase remained | |
| | | associated to analyte | |
| Sortase | Peptide | Addition of a moiety to a | Affinity reagent |
| | | peptide containing a sortase | specific to sortase- |
| | | recognition amino acid | modified peptide |
| | | sequence; the moiety to be | |
| | | added may be present in | |
| | | solution with the sortase | |
| | | modifying agent | |

Further provided herein is a composition, comprising: (a) a plurality of binding reagents, wherein each binding reagent of the plurality of binding reagents is joined with a binding reagent identifier moiety, (b) a plurality of analytes, wherein each analyte of the plurality of analytes is joined with a unique analyte identifier moiety, wherein no two analytes are joined to unique analyte identifier moieties having a same residue sequence, and (c) a plurality of polymer strands (e.g., nucleic acid strands, peptide strands, combinations thereof), wherein each polymer strand of the plurality of polymer strands comprises a residue sequence (e.g., a nucleotide sequence, an amino acid sequence) from a unique analyte identifier moiety, wherein no two polymer strands comprise a same residue sequence from an analyte identifier moiety, and optionally wherein each polymer strand of the plurality of polymer strands comprises a residue sequence from the binding reagent identifier moiety. In some cases, the plurality of polymer strands may be attached to the plurality of binding reagents and/or the plurality of analytes. In particular cases, the plurality of polymer strands may be attached to the plurality of binding reagents and the plurality of analytes. In other cases, the plurality of polymer strands may be dissociated from the plurality of binding reagents and the plurality of analytes.

After dissociating an interaction identification moiety from a binding reagent, an analyte, or a complex thereof, a method may further comprise separating a fluid phase containing the interaction identification moiety from the binding reagent, analyte, or complex. In some cases, separating the fluid phase containing the interaction identification moiety from the binding reagent, analyte, or complex can comprise removing the fluid phase containing the interaction identification moiety from a vessel containing the binding reagent, analyte, or complex. A method may further comprise transferring the interaction identification moiety to a detection device that is configured to detect the analyte-specific code. Accordingly, a system can comprise a fluidic system that is in fluidic communication with a detection device and a vessel comprising a binding reagent, an analyte, or a complex thereof.

As an alternative to methods comprising a step of forming an interaction identification moiety, some methods may comprise providing a set of pre-formed interaction identification moieties to a plurality of complexes formed by binding reagents and analytes. Each interaction identification moiety of a set of interaction identification moieties may differ from the other interaction identification moieties with respect to an analyte-specific code to which it is configured to couple. Accordingly, only interaction identification moieties of the set of interaction identification moieties will bind to the binding reagent-analyte complexes, while the remaining interaction identification moieties corresponding non-complexed analytes will remain unbound. Identical sets of pre-formed interaction identification moieties may be used in multiple cycles of an assay containing serial binding of binding reagents to analytes. If two binding reagents are multiplexed (e.g., simultaneously contacted to a population of analytes), two sets of interaction identification moieties may be provided, each set configured to couple to a unique binding reagent identifier moiety of one of the two binding reagents.

Further provided herein is a method of observing binding interactions between a plurality of affinity reagents and a population of analytes, comprising: (a) binding affinity reagents of the plurality of affinity reagents to analytes of the population of analytes to form a plurality of affinity reagent-analyte complexes, wherein each affinity reagent is individually joined with an affinity reagent identifier moiety, and wherein each analyte is individually joined with an analyte identifier moiety, wherein each analyte identifier moiety comprises a unique analyte-specific code, and wherein each affinity reagent-analyte complex comprises a single analyte coupled to a single affinity reagent, (b) after forming the affinity reagent-analyte complexes, binding to the affinity reagent-analyte complexes a plurality of interaction identification moieties, wherein each interaction identification moiety of the plurality of interaction identification moieties comprises a moiety that couples to the affinity reagent identifier moiety and a moiety that couples to the unique analyte-specific code of the analyte identifier moiety, (c) removing unbound interaction identification moieties from the plurality of affinity reagent-analyte complexes, (d) after removing the unbound interaction identification moieties, dissociating interaction identification moieties from the plurality of affinity reagent-analyte complexes; and (d) after dissociating the interaction identification moieties, detecting for each dissociated interaction identification moiety the analyte-specific code of the interaction identification moiety, thereby detecting the binding interaction between the affinity reagents and the analytes.

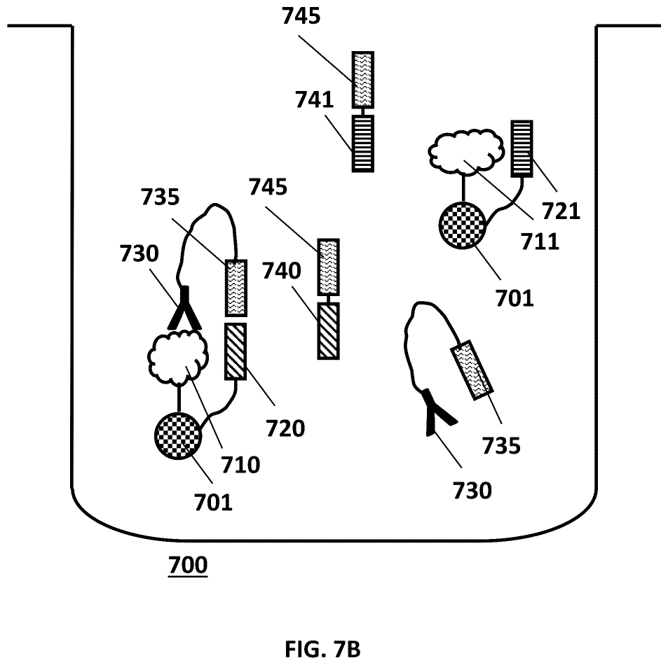

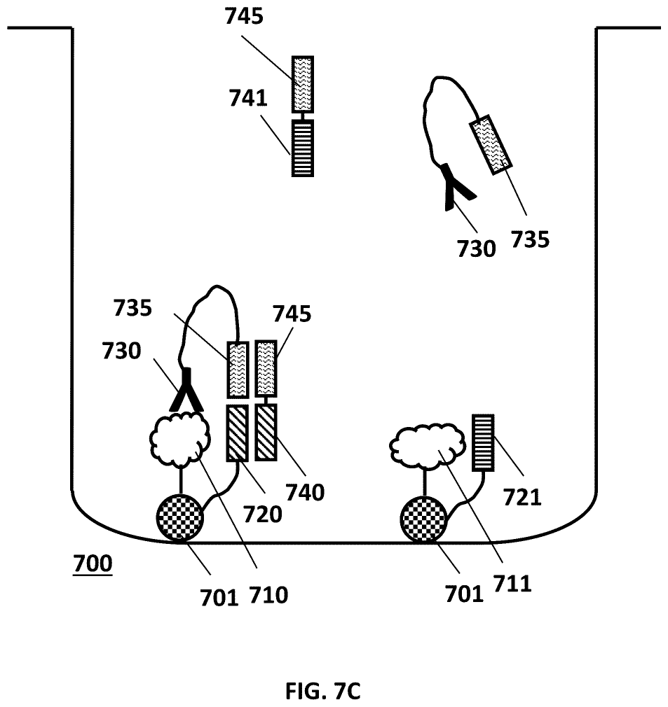

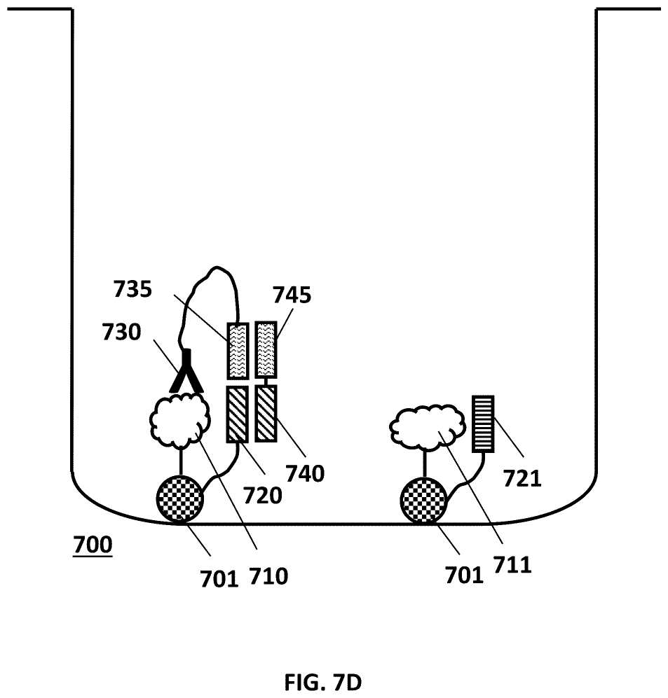

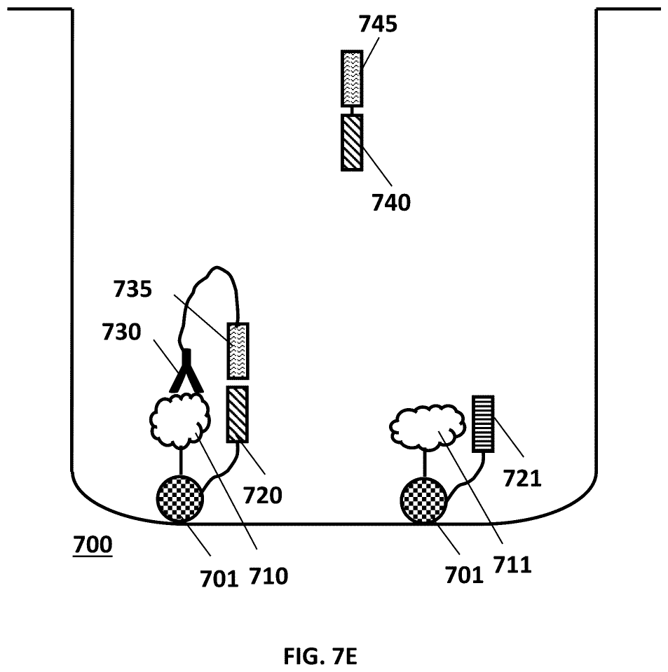

FIGS. 7A-7E illustrate a method of detecting a binding interaction utilizing pre-formed interaction identification moieties. FIG. 7A illustrates a vessel 700 comprising a plurality of analytes and a plurality of binding reagents in a fluid phase. Each of the analytes (710, 711) is attached to a solid support 701. Analyte 710 is joined to analyte identifier moiety 720, and analyte 711 is joined to analyte identifier 721. Each affinity reagent 730 is joined to an affinity reagent identifier moiety 735. FIG. 7B depicts a second configuration, in which an affinity reagent 730 has bound to analyte 710, thereby bringing its affinity reagent identifier moiety 735 in proximity to analyte identifier moiety 720. Analyte 721 is not bound by an affinity reagent 730. A plurality of interaction identification moieties has been delivered to the vessel 700. Each interaction identification moiety comprises a first moiety 745 that is configured to couple to the affinity reagent identifier moiety 735. A first interaction identification moiety further comprises a second moiety 740 that is configured to couple to the analyte identifier moiety 720 joined to analyte 710. A second interaction identification moiety further comprises a second moiety 741 that is configured to couple to the analyte identifier moiety 721 joined to analyte 711.

FIG. 7C depicts a configuration in which the solid support 701 has been transferred to a surface of the vessel 700. This transfers the affinity reagent-analyte complex containing analyte 710 and the affinity reagent 730 and the coupled first interaction identification moiety, as well as the unbound analyte 711 to the surface of the vessel. The second interaction identification moiety and an unbound affinity reagent 730 remain in the fluid phase. FIG. 7D depicts a configuration in which the fluid phase has been removed, thereby removing the second interaction identification moiety and the unbound affinity reagent 730. FIG. 7E depicts a final configuration in which the first interaction identification moiety has been dissociated from the complex containing the analyte 710 and the affinity reagent 730. Subsequently, a fluid phase containing the first interaction identification moiety may be transferred to a detection device, thereby detecting the analyte-specific code corresponding to the analyte identifier moiety 720 associated with analyte 710.

The skilled person will readily recognize variations of the method illustrated in FIGS. 7A-7E. For example, the set of interaction identification moieties may be delivered to the vessel 700 with the affinity reagents 730. Alternatively, the set of interaction identification moieties may be delivered to the vessel 700 after the affinity reagents 730 have been delivered to the vessel. If the set of interaction identification moieties is delivered after the affinity reagents 730, the method may further comprise a step of removing unbound affinity reagents 730 from the vessel 700. The depicted method of transferring a solid support 701 to a surface of the vessel 700 may facilitate removal of unbound affinity reagents (removal of unbound affinity reagents may also occur in methods involving forming interaction identification moieties). Further, it will be recognized that various method steps provided herein may be useful to the method of FIG. 7A-7E, such as methods of dissociating interaction identification moieties, and methods of transferring solid supports to vessel surfaces.

After forming an interaction identification moiety, the interaction identification moiety may be detected, thereby detecting an occurrence between a binding reagent and an analyte. Detecting an interaction identification moiety may comprise detecting an analyte-specific code, thereby determining which analyte of a population of analytes formed a binding interaction with a binding reagent. Detecting an interaction identification moiety may further comprise detecting a binding reagent-specific code, a vessel-specific code, or an assay sequence-specific code, thereby determining which binding reagent formed a binding interaction with the analyte associated with the analyte-specific code, or when during an assay the binding interaction occurred, respectively. Sequencing of polymer strands (e.g., nucleic acid strands, peptide strands, polysaccharide strands) may be a particularly useful method of detection due to the commercial availability of synthesized nucleic acids or peptides that can be utilized as components of identifier moieties, as well as the commercial availability of sequencing devices that facilitate detection of coded polymer residue sequences.

A method may comprise a step of detecting an analyte-specific code of an interaction identification moiety, in which detecting the analyte-specific code comprises sequencing a portion of the interaction identification moiety. Sequencing a portion of the interaction identification moiety can comprise sequencing a nucleotide sequence containing the analyte-specific code of the interaction identification moiety. Optionally, sequencing a portion of the interaction identification moiety can comprise sequencing a nucleotide sequence containing the binding reagent-specific code, vessel-specific code, and/or assay sequence-specific code of the interaction identification moiety. Sequencing a portion of the interaction identification moiety can comprise sequencing an amino acid sequence containing the analyte-specific code of the interaction identification moiety. Optionally, sequencing a portion of the interaction identification moiety can comprise sequencing an amino acid sequence containing the binding reagent-specific code, vessel-specific code, and/or assay sequence-specific code of the interaction identification moiety.

An interaction identification moiety can be detected with a detection device after a binding interaction. Interaction detection moieties comprising polymer strands may be particularly useful as one or more code sequences (e.g., an analyte-specific code, a binding reagent-specific code, a vessel-specific code, an assay sequence-specific code) of the polymer strand can be detected with a sequencing device. Accordingly, interaction identification moieties comprising nucleic acid code sequences may be detected on a sequencing device. Methods and systems for the sequencing of nucleic acids are well known in the art, including array- or bead-based sequencing methods (e.g., Illumina, Pacific Biosciences), and nanopore-based sequencing (e.g., Oxford Nanopore Technologies). Likewise, interaction identification moieties comprising peptide sequences may be detected on a peptide sequencing device. The section below titled “Single-Analyte Assays” describes several methods and systems for peptide sequencing, including fluorosequencing (e.g., systems developed by Erisyon) and binding reagent-based sequencing (e.g., systems developed by Encodia, Quantum-Si, etc.).

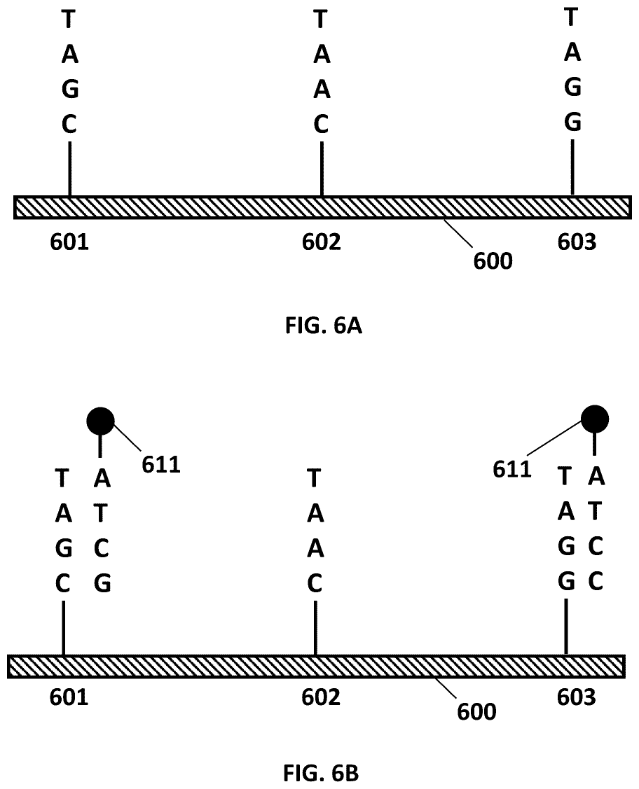

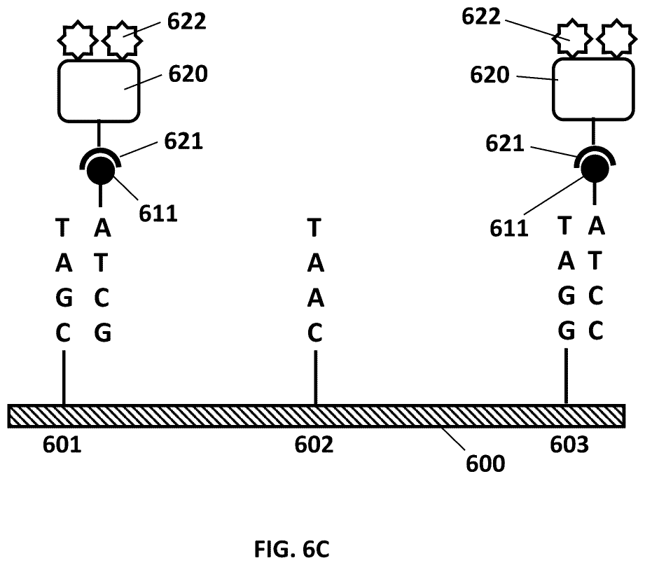

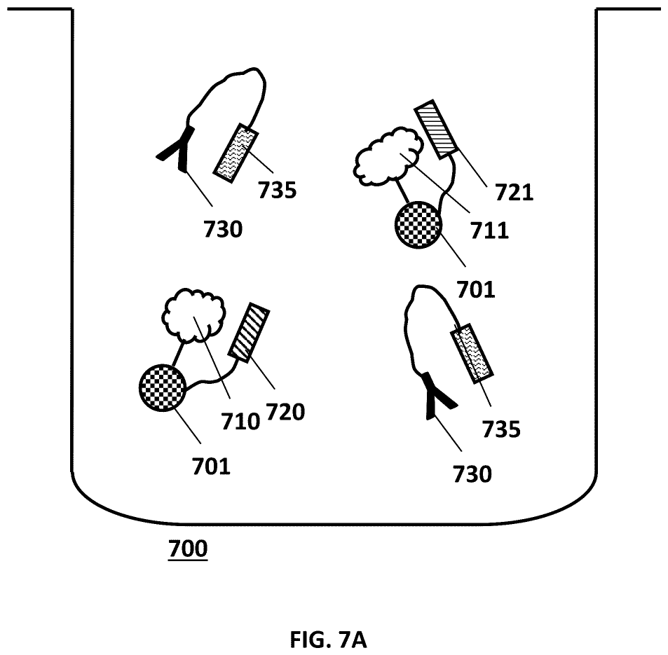

Alternatively, interaction identification moieties comprising nucleic acid code sequences may be detected on an array-based detection device. FIGS. 6A-6C depict a scheme for capturing and detecting interaction identification moieties on an array-based detection device. FIG. 6A illustrates a solid support 600 comprising three unique, optically resolvable sites (601, 602, 603). Each site contains a different attached capture oligonucleotide. The capture oligonucleotide contains a unique nucleotide sequence that is complementary to the nucleotide sequence of an analyte-specific code. In some configurations, each site may contain a plurality of duplicate copies of the capture oligonucleotide. FIG. 6B depicts a configuration of the array-based detection device after the device has been contacted with a plurality of interaction identification moieties. Each interaction identification moiety comprises an analyte-specific code and a detectable label 611 (e.g., a biotin group attached by biotin ligase labeling, a moiety attached by a sortase enzyme, etc.). Sites 601 and 603 have bound to interaction identification moieties due to base pair coupling of the analyte-specific codes to the unique, complementary capture oligonucleotides. FIG. 6C depicts a final configuration in which fluorescent detection reagents have been coupled to the detectable labels 611 of the interaction identification moieties. The fluorescent detection reagents each comprise a coupling moiety 621 that binds to the detectable label 611, and one or more fluorescent labels 621, optionally attached by a retaining component (e.g., a nanoparticle, a nucleic acid particle, etc.). Occupied array sites 601 and 603 can be detected, thereby indicating that interaction identification moieties for the corresponding analyte-specific codes were captured. Likewise, no signal will be detected at site 602, indicating that no interaction identification moiety for that analyte-specific code was captured. The result can be correlated to a binding interaction occurring for analytes joined with the analyte-specific codes captured by sites 601 and 603. Subsequently, the captured interaction identification moieties can be dissociated. The detection device can be reused for multiple rounds of binding to a population of analytes. Each detection event at a same site of the array would indicate a newly detected binding interaction for a single analyte. Likewise, the device can be reused for multiple populations of analytes (e.g., populations in separate vessels) provided a common set of analyte-specific codes is used for each individual population of analytes.

In addition to the foregoing reagents, also provided herein are kits useful in carrying out the analyses described herein, which kits may include the affinity reagents described above. The kits may optionally include one or more of enrichment reagents used to enrich for low abundance proteins and proteoforms, e.g., beads and antibodies used for the immune-isolation and/or immunoprecipitation of the proteins of interest, wash and other elution reagents, for such enrichment. Such kits may also include the vessels and/or particles used to immobilize proteins of interest in a single molecule, in a detectable format for subsequent analysis in appropriately configured detection systems described herein. Such kits can include instructions for carrying out the enrichment, flow-cell deposition, interrogation, and follow-on analysis of biological samples using such kits.

Exemplary kit components may include one or more of: (i) pluralities of particles containing analyte identification moieties, (ii) beads or other solid supports for immobilization of analytes, (iii) sets of affinity reagents for analyses set forth herein, (iv) vessels (e.g., multi-well plates) for conducting assays set forth herein, and (v) additional media for performing methods set forth herein.

One or more compositions set forth herein can be provided in kit form including, if desired, a suitable packaging material. Optionally, one or more compositions can be provided as a solid, such as crystals or a lyophilized pellet. Accordingly, any combination of reagents or components that is useful in a method set forth herein can be included in a kit.

The packaging material included in a kit can include one or more physical structures used to house the contents of the kit. The packaging material can be constructed by well-known methods, preferably to provide a sterile, contaminant-free environment. The packaging materials employed herein can include, for example, those customarily utilized in affinity reagent systems. Exemplary packaging materials include, without limitation, glass, plastic, paper, foil, and the like, capable of holding within fixed limits a component useful in the methods of the present disclosure.

Packaging material or other components of a kit can include a kit label which identifies or describes a particular method set forth herein. For example, a kit label can indicate that the kit is useful for detecting a particular protein or proteome. In another example, a kit label can indicate that the kit is useful for a therapeutic or diagnostic purpose, or alternatively that it is for research use only.

Instructions for use of the packaged reagents or components are also typically included in a kit. The instructions for use can include a tangible expression describing the reagent or component concentration or at least one assay method parameter, such as the relative amounts of kit components and sample to be admixed, maintenance time periods for reagent/sample admixtures, temperature, buffer conditions, and the like.

In some cases, a kit can be configured as a cartridge or component of a cartridge. The cartridge can in turn be configured to be engaged with a detection apparatus. For example, the cartridge can be engaged with a detection apparatus such that contents of the cartridge are in fluidic communication with the detection apparatus or with a vessel engaged with the detection apparatus. A cartridge can be engaged with a detection apparatus such that contents of the cartridge can be observed by the detection apparatus, for example, using an assay set forth herein.

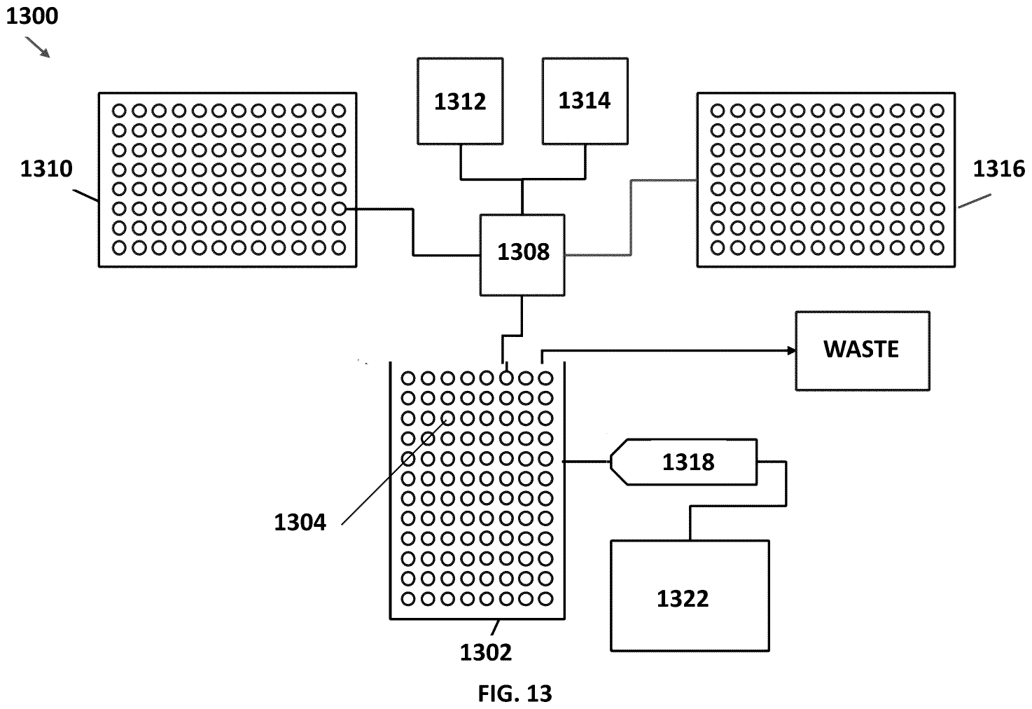

Additionally, provided herein are systems for performing the techniques, reagents, systems, and methods described herein. An example of a system is illustrated in FIG. 13. As shown, the system 1300 includes one or more vessels (e.g., a well plate) 1302 that includes a plurality of fluidically isolated vessels in which individual protein molecules from a sample may be deposited.

The system will also typically include a fluidic delivery system 1308 that is configured to deliver different fluids to one or more vessels 1302 through a series of fluidic lines and utilizing appropriate pumps, valves and other conventional fluid controls. The fluidics system 1308 may be fluidically coupled to various sources of fluids and reagents needed to carry out the analysis on the flow cell. For example, as shown, fluidic system 1308 is fluidly coupled to a source of a plurality of reagents 1310 (shown as a 96 well plate, although any number of different reagent storage systems of varying capacity may be employed) that includes a library of multiple affinity reagents that each have affinity for different characteristics of one or more proteins of interest. Additionally, fluidic system 1308 may also be coupled to sources of washing fluids or buffers 1312, and removal reagents 1314 (for removing bound affinity reagents following detection), as well as any other ancillary fluids and reagents needed for the analysis. Similarly, the fluidic system may be coupled to sources of different sample materials that are to be analyzed 1316 (again, shown as a 96 well plate, although again, any suitable sample storage system or capacity may be suitable).

The reagents sources are typically fluidly connected to the flow-cell using fluidics systems that can separately access different reagents, sample materials and other fluids, and control the timing and volume of different reagents delivered to the flow-cell at different times in order to carry out the deposition, interrogation, washing and removal steps of the analysis process. Such fluidic systems will typically include requisite valves and pumps for carrying out such fluid deliveries and include, for example, those as described in, for example, International Patent Application No. WO 2023/122589A2, the full disclosure of which is hereby incorporated herein by reference in its entirety for all purposes.

The systems described herein also typically include a detection system, such as a sequencing device 1318, for detecting and recording interaction identification moieties, as set forth herein. The sequencing device can be fluidically coupled to the fluidics system 1308, thereby facilitating transfer of interaction identification moieties from the one or more vessels 1302 to the sequencing device. Any suitable sequencing device can be utilized, including devices that utilize sequencing by synthesis, sequencing by ligation, or pore-based sequencing. A system can further comprise one or more separate vessels (not shown) that are configured to separate interaction identification moieties from other assay reagents before delivery of the interaction identification moieties to a detection device.

The overall systems also typically include one or more computers or processors 1322 for controlling the operation of the instrument system including the fluidic system 1308 (e.g., to sample different sample sources 1316, reagent sources 1310 and delivery timing and volume of each), and detection system 1318, among other functions, and for recording the detected signals received from the detection system 1318, e.g. fluorescent signals, and analyzing such signals to identify potential binding by each of the different affinity reagents. Processors 1322 also have access to memory storing instructions that are executed to perform any of the techniques described herein. Included in such memory may be bioinformatic software or firmware that evaluates the signals received and based upon appropriate modeling, identifies likely positive binding events, and then subsequently provides an overall assessment of characteristics of the proteins as described herein including identification information of proteins that are present at any given location on the array and/or the relative abundance of each different protein across the array and ultimately, within the sample being analyzed. Examples of bioinformatic software processes for analyzing such proteoform and proteome data have been describe in, for example, U.S. Pat. Nos. 11,545,234, 10,473,654B1, U.S. Provisional Patent Application No. 63/761,498, and Egertson, et al., “A theoretical framework for proteome-scale single-molecule protein identification using multi-affinity protein binding reagents,” bioRxiv, https://doi.org/10.1101/2021.10.11.463967, U.S. Patent Application No. 2022/0236282, International Patent Application Nos. PCT/US24/15132, and WO 2023/038859, each of which is herein incorporated by reference in its entirety. Alternatively, in some cases, recorded data from the binding events, stored as digital information, digital image files, or compressed versions of such image files, may be transmitted to separate servers or cloud-based systems, which house the informatics software that performs this latter analysis and reporting.

The computer system 1322 can be an electronic device of a detection system, the electronic device being integral to the detection system or remotely located with respect to the detection system. The computer system 1322 includes a computer processing unit (CPU, also “processor” and “computer processor” herein), which can be a single core or multi-core processor, or a plurality of processors for parallel processing. The computer system 1322 also includes memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus (solid lines), such as a motherboard. The storage unit can be a data storage unit (or data repository) for storing data. The computer system 1322 can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. For example, one or more computer servers may enable cloud computing over the network (“the cloud”) to perform various aspects of analysis, calculation, and generation of the present disclosure, such as, for example, receiving information of empirical measurements of analytes in a sample; processing information of empirical measurements against a database comprising a plurality of candidate analytes, for example, using a binding model or function set forth herein; generating probabilities of a candidate analytes generating empirical measurements, and/or generating probabilities that extant analytes are correctly identified in the sample, and/or determining abundances of analytes in the sample. Such cloud computing may be provided by cloud computing platforms such as, for example, Amazon Web Services (AWS), Microsoft Azure, Google Cloud Platform, and IBM cloud. The network, in some cases with the aid of the computer system 1322, can implement a peer-to-peer network, which may enable devices coupled to the computer system 1322 to behave as a client or a server.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system 1322 can be included in the circuit. In some cases, the circuit is an application specific integrated circuit (ASIC).

The storage unit can store files, such as drivers, libraries and saved programs. The storage unit can store user data, e.g., user preferences and user programs. The computer system 1322, in some cases, can include one or more additional data storage units that are external to the computer system 1322, such as located on a remote server that is in communication with the computer system 1322 through an intranet or the Internet.

The computer system 1322 can communicate with one or more remote computer systems through the network. For instance, the computer system 1322 can communicate with a remote computer system of a user. Examples of remote computer systems include personal computers (e.g., portable PC), slate or tablet PC's (e.g., Apple® iPad, Samsung® Galaxy Tab), telephones, smartphones (e.g., Apple® iphone, Android-enabled device, Blackberry®), or personal digital assistants. The user can access the computer system 1322 via the network.

Methods as described herein can be implemented by way of machine (e.g., computer processor) executable code stored on an electronic storage location of the computer system 1322, such as, for example, on the memory or electronic storage unit. The machine executable or machine readable code can be provided in the form of software. During use, the code can be executed by the processor. In some cases, the code can be retrieved from the storage unit and stored in the memory for ready access by the processor. In some situations, the electronic storage unit can be precluded, and machine-executable instructions are stored on memory.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 1322, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1322 can include or be in communication with an electronic display that comprises a user interface (UI) for providing, for example, user selection of algorithms, binding measurement data, candidate proteins, and databases. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit. The algorithm can, for example, receive information of empirical measurements of extant proteins in a sample, compare information of empirical measurements against a database comprising a plurality of protein sequences corresponding to candidate proteins, generate probabilities of a candidate protein generating the observed measurement outcome profile, and/or generate probabilities that candidate proteins are correctly identified in the sample, and/or generate abundances for the proteins in the sample.

The present disclosure provides a non-transitory information-recording medium that has, encoded thereon, instructions for the execution of one or more steps of the methods or techniques set forth herein, for example, when these instructions are executed by an electronic computer in a non-abstract manner. This disclosure further provides a computer processor (i.e. not a human mind) configured to implement, in a non-abstract manner, one or more of the methods set forth herein. All methods, compositions, devices and systems set forth herein will be understood to be implementable in physical, tangible and non-abstract form. The claims are intended to encompass physical, tangible and non-abstract subject matter. Explicit limitation of any claim to physical, tangible and non-abstract subject matter, will be understood to limit the claim to cover only non-abstract subject matter, when taken as a whole. Reference to “non-abstract” subject matter excludes and is distinct from “abstract” subject matter as interpreted by controlling precedent of the U.S. Supreme Court and the United States Court of Appeals for the Federal Circuit as of the priority date of this application.

Further provided herein is a system, comprising: (a) a population of polymer strands (e.g., nucleic acid strands, peptide strands, combinations thereof), wherein each polymer strand comprises a unique analyte identifier residue sequence, and optionally a binding reagent identifier residue sequence, (b) a detection device (e.g., a sequencing device), wherein the sequencing device is configured to detect for each polymer strand of the population of polymer strands the residue sequence of the polymer strand, and (c) a processor configured to: (i) receive sequencing data from the sequencing device, wherein the sequencing data comprises for each polymer strand of the population of polymer strands a residue sequence, (ii) based on the sequencing data, detect for each polymer strand of the population of polymer strands the unique analyte identifier residue sequence, and optionally the binding reagent identifier residue sequence, and (iii) record in a binding profile a presence of binding of a binding reagent to an analyte associated with each detected unique analyte identifier residue sequence, and optionally record in the binding profile binding reagent information from the binding reagent identifier residue sequence.

In some cases, a system, as set forth herein, may comprise a nucleic acid sequencing device. In other cases, a system, as set forth herein, may comprise a peptide sequencing device.

A system may further comprise: (d) a vessel comprising one or more analytes, wherein each analyte of the one or more analytes is joined with a unique analyte identifier moiety comprising a unique analyte identifier residue sequence, in which no two analytes are paired to the same analyte identifier moiety, and in which no two analyte identifier moieties comprises the same unique analyte identifier residue sequence, (e) a library of binding reagents, wherein the library of binding reagents comprises a plurality of pools of binding reagents, the binding specificity of each pool of the plurality of pools of binding reagents differing from the binding specificity of other pools of the plurality of pools of binding reagents, and (f) a fluid transfer device that is configured to: (i) transfer a pool of binding reagents from the library of binding reagents to the vessel, and (ii) transfer the population of polymer strands from the vessel to the detection device. In some cases, the fluid transfer device may comprise an automated pipetting device.

The skilled person will readily recognize that certain method steps disclosed herein may require providing or delivering to an analyte, a binding reagent, an interaction identification moiety, or a vessel comprising any of these entities one or more additional reagents. For example, certain methods have disclosed use of enzymes such as nucleic acid ligases, biotin ligases, sortases, and polymerases. Accordingly, a system may further comprise a library of reagents, wherein the library of reagents comprises one or more reagents selected from the group consisting of: (i) an enzyme, (ii) an enzyme substrate, (iii) a binding reagent dissociation medium, (iv) a rinsing medium, and (v) a non-enzymatic modifying agent. A system may comprise a fluid transfer device that is configured to transfer the one or more reagents from the library of reagents to a vessel containing an analyte, a binding reagent, and/or an interaction identification moiety.

In some configurations, a system may comprise a plurality of vessels, in which a population of analytes can be distributed amongst the vessels of the plurality of vessels. In some configurations, a system may comprise a plurality of vessels, in which a population of analytes can be placed in each vessel of the plurality of vessels. For example, each vessel of a plurality of vessels may be provided a population of analytes from a different sample source. A system may comprise a fluid transfer device that is configured to transfer an analyte or a population thereof to one or more vessels of a system, as set forth herein. A system may further comprise a fluid transfer device that is configured to: (i) transfer a pool of binding reagents from a library of binding reagents to each vessel of the plurality of vessels, and (ii) transfer a population of interaction identification moieties (e.g., polymer strands) from a vessel, or each vessel of the plurality of vessels, to a detection device (e.g., a sequencing device).

An advantage of distributing a population of analytes amongst a plurality of vessels is that it may reduce the quantity of unique code sequences that are necessary to perform a method set forth herein. For example, if a population of analytes containing about 10,000,000,000 analytes is distributed equally into vessels of a 384-well plate, each vessel will contain about 26,000,000 analytes. Accordingly, only about 26,000,000 unique analyte-specific codes would be necessary to distinguish each analyte in each well. Oligonucleotide barcodes having a length of 13 nucleotides contain sufficient information space to provide at least 26,000,000 unique analyte-specific codes. Different analytes joined to analyte identifier moieties containing identical analyte-specific codes could be distinguished by: i) staged detection of interaction identification moieties (e.g., individually detecting populations of interaction identification moieties from each vessel until all vessels have been detected); or ii) including a vessel-specific code in analyte identifier moieties, thereby facilitating pooled detection of interaction identification moieties from multiple vessels simultaneously.

Further reductions in the number of necessary unique analyte-specific codes may be achieved in systems that utilize droplet-based methods. Co-location of analytes, binding reagents, and other assay components in droplets can form isolated systems that don't favor exchange between droplets. Sequential processing of interaction identification moieties from each droplet can allow analyte-specific codes to be used commonly among different droplets. In the above-described example of a 384-well plate, if the analyte contents of each well are distributed among 1000 unique droplets, only 26,000 unique analyte-specific codes are necessary. Oligonucleotide barcodes having a length of 8 nucleotides contain sufficient information space to provide at least 26,000 unique analyte-specific codes. Droplet-based methods may be especially useful for methods utilizing multiplexed affinity reagents, such as “one-pot” methods described herein. Accordingly, in some configurations, method steps set forth herein may occur within one or more droplets within a vessel, as set forth herein. Methods of partitioning analytes and other reagents into droplets are described in U.S. Patent Publication Nos. 20140155295A1, 20150292988A1, and 20150376609A1, each of which is incorporated herein by reference in its entirety.

Certain disclosed methods may be facilitated by a system that includes a field-generating device. Exemplary field-generating devices can include magnetic field generators (e.g., rare-earth magnets, electromagnets), electric field generators, and thermal controllers (e.g., Pelletier devices). A system may include a light-field producing device, such as a laser, a lamp, a filament, or a light-emitting diode.

After a binding reagent has bound to an analyte, a method may further comprise dissociating the binding reagent from the analyte. Preferably, a complex comprising a binding reagent and an analyte is dissociated after sufficient time has elapsed for an interaction identification moiety to be formed, thereby recording the binding interaction. A binding reagent may be dissociated from an analyte before or after an interaction identification moiety has been separated from the binding reagent, analyte, or complex thereof. In some cases, dissociating the binding reagent from the analyte may comprise: (i) dissociating the binding reagent and the interaction identification moiety from the analyte; and (ii) after dissociating the binding reagent and the interaction identification moiety from the analyte, separating the binding reagent from the interaction identification. In other cases, dissociating the binding reagent from the analyte may comprise: (i) separating the interaction identification moiety from the binding reagent and the analyte; and (ii) after separating the interaction identification moiety from the binding reagent and the analyte, dissociating the binding reagent from the analyte. A binding reagent may be dissociated from an analyte by any suitable method including altering the temperature of a fluid phase containing the binding reagent and/or analyte, altering the pH of a fluid phase containing the binding reagent and/or analyte, or providing a fluid phase containing a dissociation agent (e.g., sodium dodecyl sulfate, guanidinium chloride, CHAPS, urea, etc.). Additional methods and compositions for dissociating a binding reagent from an analyte are provided in US 2024/0192202A1, which is herein incorporated by reference in its entirety.

In some cases, after dissociating an interaction identification moiety and/or separating the interaction identification moiety from an analyte, or a complex containing the analyte, the analyte may no longer be joined to an analyte identifier moiety. FIGS. 5A-5C depict various configurations of interaction identification moieties containing at least a portion of an analyte identifier moiety. Accordingly, the analyte identifier moiety is partially or wholly removed by dissociation of the interaction identification moiety. If an analyte is to be incubated with additional binding reagents after losing its analyte identifier moiety, a method may further comprise providing and/or joining a second analyte identifier moiety to the analyte. In some cases, an analyte may be joined to a first oligonucleotide, in which an analyte identifier moiety comprising a second oligonucleotide is joined to the analyte by hybridizing the first oligonucleotide to the second oligonucleotide. In some cases, dissociation of an interaction identification moiety may form a reactive or non-reactive moiety where a prior analyte identifier moiety had been attached. For example, cleavage of a photolabile compound may provide a reactive functional group. In another example, cleavage of a nucleic acid may provide a partially single-stranded or partially-double stranded nucleic acid. A method may comprise attaching a second analyte identifier moiety to a reactive or non-reactive moiety.

An assay may be performed by contacting an analyte with a sequence or series of binding reagents. Each instance during an assay of contacting an analyte with a binding reagent may be paired with a step of detecting a presence or absence of an interaction identification moiety formed due to a binding interaction between the analyte and the binding reagent. In some cases, an assay may comprise two or more steps of contacting an analyte with a same binding reagent (with respect to binding specificity). In other cases, an assay may comprise two or more steps of contacting an analyte with two or more binding reagents, each of the two or more binding reagents differing with respect to binding specificity. In some cases, an assay may comprise two or more steps of contacting an analyte with two or more binding reagents, each of the two or more binding reagents differing with respect to the binding reagent identifier moiety (e.g., each differing binding reagent having a different binding reagent-specific code). In some cases, a method may further comprise determining a binding profile for an analyte, in which the binding profile comprises data concerning the presence or absence of a detected interaction identification moiety containing the analyte-specific code associated with the analyte for each of two or more binding reagents.

A method may further comprise contacting a second binding reagent to the analyte. In some cases, a method may further comprise repeating one or more steps set forth herein with the second binding reagent, thereby detecting a second interaction identification moiety comprising the analyte-specific code. Accordingly, a binding profile may be formed for the analyte comprising the presence of binding for a first binding reagent and a second binding reagent. In other cases, a method may further comprise repeating one or more steps set forth herein with the second binding reagent, thereby not detecting a second interaction identification moiety comprising the analyte-specific code. Accordingly, a binding profile may be formed for the analyte comprising the presence of binding for a first binding reagent and the absence of binding for a second binding reagent. A method may further comprise, based upon a binding profile for an analyte, characterizing the analyte. In some cases, characterizing an analyte may comprise determining an identity of the analyte (e.g., a species of analyte). In some cases, characterizing an analyte may comprise determining a proteoform of the analyte.

The present disclosure provides compositions, apparatus and methods for detecting one or more proteins. A protein can be detected using one or more affinity agents having binding affinity for the protein. The affinity agent and the protein can bind each other to form a complex and, during or after formation, the complex can be detected. The complex can be detected directly, for example, due to a label that is present on the affinity agent or protein. In some configurations, the complex need not be directly detected. For example, complex formation can yield a chemical change, such as formation of a nucleic acid tag, that is detected after the complex has been formed and in some cases after the complex has been dissociated.

The present disclosure provides compositions, apparatus and methods that can be useful for characterizing analytes, such as proteins, by obtaining multiple separate and non-identical measurements of the analytes. In particular configurations, the individual measurements may not, by themselves, be sufficiently accurate or specific to make the characterization, but in combination, the multiple non-identical measurements can allow the characterization to be made with a high degree of accuracy, specificity and confidence. For example, the multiple separate measurements can include subjecting a sample to reagents that are promiscuous with regard to recognizing a variety of different analytes that are present in the sample. Accordingly, a first measurement carried out using a first promiscuous reagent may perceive a first subset of the analytes without distinguishing different analytes within the subset. A second measurement carried out using a second promiscuous reagent may perceive a second subset of analytes, again, without distinguishing one analyte in the second subset from other analytes in the second subset. However, a comparison of the first and second measurements can distinguish: (i) an analyte that is uniquely present in the first subset but not the second; (ii) an analyte that is uniquely present in the second subset but not the first; (iii) an analyte that is uniquely present in both the first and second subsets; or (iv) an analyte that is uniquely absent in the first and second subsets. The number of promiscuous reagents used, the number of separate measurements acquired, and the degree of reagent promiscuity (e.g. the diversity of components recognized by the reagent) can be adjusted to suit the diversity of analytes expected for a particular sample.

The present disclosure provides assays that are useful for detecting one or more analytes. Exemplary assays are set forth herein in the context of detecting proteins. Those skilled in the art will recognize that methods, compositions and apparatus set forth herein can be adapted for use with other analytes such as cells, organelles, nucleic acids, polysaccharides, metabolites, vitamins, hormones, enzyme co-factors, therapeutic agents, candidate therapeutic agents and others set forth herein or known in the art. Particular configurations of the methods, apparatus and compositions set forth herein can be made and used, for example, as set forth in U.S. Pat. Nos. 10,473,654 or 11,282,585; US Pat. App. Pub. Nos. 2020/0082914A1 or 2023/0114905A1; or Egertson et al., BioRxiv (2021), DOI: 10.1101/2021.10.11.463967, each of which is incorporated herein by reference. Exemplary methods, systems and compositions are set forth in further detail below.

A composition, apparatus or method set forth herein can be used to characterize an analyte, or moiety thereof, with respect to any of a variety of characteristics or features including, for example, presence, absence, quantity (e.g. amount or concentration), chemical reactivity, molecular structure, structural integrity (e.g. full length or fragmented), maturation state (e.g. presence or absence of pre- or pro-sequence in a protein), location (e.g. in an analytical system, subcellular compartment, cell or natural environment), association with another analyte or moiety, binding affinity for another analyte or moiety, biological activity, chemical activity or the like. An analyte can be characterized with regard to a relatively generic characteristic such as the presence or absence of a common structural feature (e.g. amino acid sequence length, overall charge or overall pKa for a protein) or common moiety (e.g. a short primary sequence motif or post-translational modification for a protein). An analyte can be characterized with regard to a relatively specific characteristic such as a unique amino acid sequence (e.g. for the full length of the protein or a motif), an RNA or DNA sequence that encodes a protein (e.g. for the full length of the protein or a motif), or an enzymatic or other activity that identifies a protein. A characterization can be sufficiently specific to identify an analyte, for example, at a level that is considered adequate or unambiguous by those skilled in the art.

In particular configurations, a method set forth herein can be used to identify a number of different extant proteins that exceeds the number of affinity reagents used. For example, the number of different protein species identified can be at least 5×, 10×, 25×, 50×, 100× or more than the number of affinity reagents used. This can be achieved, for example, by (1) using promiscuous affinity reagents that bind to multiple different candidate proteins suspected of being present in a given sample, and (2) subjecting the extant proteins to a set of promiscuous affinity reagents that, taken as a whole, are expected to bind each candidate protein in a different combination, such that each candidate protein is expected to generate a unique profile of binding and non-binding events when subjected to the set. Promiscuity of an affinity reagent can arise due to the affinity reagent recognizing an epitope that is known to be present in a plurality of different candidate proteins. For example, epitopes having relatively short amino acid lengths, such as dimers, trimers, tetramers or pentamers, are expected to occur in a substantial number of different proteins in a typical proteome. Alternatively or additionally, a given promiscuous affinity reagent may recognize multiple different epitopes (e.g. epitopes differing from each other with regard to amino acid composition or sequence). For example, a promiscuous affinity reagent that is designed or selected for its affinity toward a first trimer epitope may also have affinity for a second epitope that has a different sequence of amino acids compared to the first epitope. Methods for screening promiscuous affinity reagents and characterizing the binding of affinity reagents to analytes is described in U.S. Pat. No. 11,970,693, which is herein incorporated by reference in its entirety.

Although performing a single binding reaction between a promiscuous affinity reagent and a complex protein sample may yield ambiguous results regarding the identity of the different extant proteins to which it binds, the ambiguity can be resolved by decoding the binding profiles for each extant protein using machine learning or artificial intelligence algorithms that are based on probabilities for the affinity reagents binding to candidate proteins. For example, a plurality of different promiscuous affinity reagents can be contacted with a complex population of extant proteins, wherein the plurality is configured to produce a different binding profile for each candidate protein suspected of being present in the population. The plurality of promiscuous affinity reagents can produce a binding profile for each extant protein that can be decoded to identify a unique combination of positive outcomes (i.e. observed binding events) and/or negative binding outcomes (i.e. observed non-binding events), and this can in turn be used to identify the extant protein as a particular candidate protein having a high likelihood of exhibiting a similar binding profile.

Binding profiles can be obtained for extant proteins and the binding profiles can be decoded or disambiguated to identify extant proteins corresponding to the binding profiles. In many cases one or more binding events produces inconclusive or even aberrant results and this, in turn, can yield ambiguous binding profiles. For example, observation of binding outcomes at single-molecule resolution can be particularly prone to ambiguities due to stochasticity in the behavior of single molecules when observed using certain detection hardware. As set forth above, ambiguity can also arise from affinity reagent promiscuity. Decoding can utilize a binding model that evaluates the likelihood or probability that one or more candidate proteins that are suspected of being present in an assay will have produced an empirically observed binding profile. The binding model can include information regarding expected binding outcomes (e.g. positive binding outcomes and/or negative binding outcomes) for one or more affinity reagents with respect to one or more candidate proteins. A binding model can include a measure of the probability or likelihood of a given candidate protein generating a false positive or false negative binding result in the presence of a particular affinity reagent, and such information can optionally be included for a plurality of affinity reagents.

Decoding can be configured to evaluate the degree of compatibility of one or more empirical binding profiles with results computed for various candidate proteins using a binding model. For example, to identify an extant protein in a sample, an empirical binding profile for the extant protein can be compared to results computed by the binding model for many or all candidate proteins suspected to be in the sample. A machine learning or artificial intelligence algorithm can be used. An algorithm used for decoding can utilize Bayesian inference. In some configurations, identity for an extant protein is determined based on a likelihood of the extant protein being a particular candidate protein, given the empirical binding pattern or based on the probability of a particular candidate protein generating the empirical binding pattern. Particularly useful decoding methods are set forth, for example, in U.S. Pat. Nos. 10,473,654 or 11,282,585; US Pat. App. Pub. Nos. 2020/0082914A1 or 2023/0114905A1; or Egertson et al., BioRxiv (2021), DOI: 10.1101/2021.10.11.463967, each of which is incorporated herein by reference. It will be recognized that the methods set forth herein that are utilized to decode extant proteins may be useful for other analyte identification assays, provided said analyte identification assays provide a binding profile that can be decoded.

In some configurations of the compositions, apparatus and methods set forth herein, one or more proteins can be present on a solid support, where the proteins can optionally be detected. For example, a protein can be attached to a solid support, the solid support can be contacted with a detection agent (e.g. affinity agent) in solution, the affinity agent can interact with the protein, thereby producing a detectable signal, and then the signal can be detected to determine the presence, absence, quantity, a characteristic or identity of the protein. In multiplexed versions of this approach, different proteins can be attached to different addresses on a solid support, and the detection steps can occur in parallel, such that proteins at each address are detected, quantified, characterized or identified. In another example, detection agents can be attached to a solid support, the support can be contacted with proteins in solution, the proteins can interact with the detection agents, thereby producing a detectable signal, and then the signal can be detected to determine the presence of the proteins.

In multiplexed configurations, different proteins can be attached to different unique identifiers (e.g. addresses in an array, analyte identifier moieties), and the proteins can be manipulated and detected in parallel. For example, a fluid containing one or more different affinity agents can be delivered to a vessel such that the proteins of the vessel are in simultaneous contact with the affinity agent(s). The total number of proteins of a sample that is detected, characterized or identified can differ from the number of different primary sequences in the sample, for example, due to the presence of multiple copies of at least some protein species. Moreover, the total number of proteins of a sample that is detected, characterized or identified can differ from the number of candidate proteins suspected of being in the sample, for example, due to the presence of multiple copies of at least some protein species, absence of some proteins in a source for the sample, or loss of some proteins prior to analysis.

A protein can be attached to a unique identifier using any of a variety of means. The attachment can be covalent or non-covalent. Exemplary covalent attachments include chemical linkers such as those achieved using click chemistry or other linkages known in the art or described in U.S. patent application Ser. No. 17/062,405, which is incorporated herein by reference. Non-covalent attachment can be mediated by receptor-ligand interactions (e.g. (strept) avidin-biotin, antibody-antigen, or complementary nucleic acid strands), for example, wherein the receptor is attached to the unique identifier and the ligand is attached to the protein or vice versa. In particular configurations, a protein is attached to a solid support (e.g. an address in an array, a particle) via a structured nucleic acid particle (SNAP). A protein can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful. The use of SNAPs and other moieties to attach proteins to unique identifiers, such as tags or addresses in an array are set forth in U.S. patents application Ser. Nos. 17/062,405 and 63/159,500, each of which is incorporated herein by reference.

A method set forth herein can be carried out in a fluid phase or on a solid phase. For fluid phase configurations, a fluid containing one or more proteins can be mixed with another fluid containing one or more affinity agents. For solid phase configurations one or more proteins or affinity agents can be attached to a solid support. One or more components that will participate in a binding event can be contained in a fluid and the fluid can be delivered to a solid support, the solid support being attached to one or more other component that will participate in the binding event. A solid support can be composed of a substrate that is insoluble in aqueous liquid. The substrate can have any of a variety of other characteristics such as being rigid, non-porous or porous. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers. In some cases, a solid support may comprise silicon, fused silica, quartz, mica, or borosilicate glass. In a particular configuration, components of a binding reaction are disposed in a vessel or a plurality of vessels, such as a 96-well or 384-well plate.

A method of the present disclosure can be carried out at single analyte resolution. As such, a single analyte (i.e. one and only one analyte), such as a single protein, can be individually manipulated or distinguished using a method set forth herein. A single analyte can be a single molecule (e.g. single protein), a single complex of two or more molecules (e.g. a single protein attached to a structured nucleic acid particle or a single protein attached to an affinity agent), a single particle, or the like. A single analyte may be resolved from other analytes based on, for example, spatial or temporal separation from the other analytes. Reference herein to a ‘single analyte’ in the context of a composition, apparatus or method does not necessarily exclude application of the composition, apparatus or method to multiple single analytes that are manipulated or distinguished individually, unless indicated to the contrary.

As an alternative to single-analyte resolution, a method can be carried out at ensemble-resolution or bulk-resolution. Bulk-resolution configurations acquire a composite signal from a plurality of different analytes or affinity agents in a vessel or on a surface. For example, a composite signal can be acquired from a population of different protein-affinity agent complexes in a well or cuvette, or on a solid support surface, such that individual complexes are not resolved from each other. Ensemble-resolution configurations acquire a composite signal from a first collection of proteins or affinity agents in a sample, such that the composite signal is distinguishable from signals generated by a second collection of proteins or affinity agents in the sample.

A composition, apparatus or method set forth herein can be configured to contact one or more analytes (e.g. an array of different proteins) with a plurality of different affinity agents. For example, a plurality of affinity agents (whether configured separately or as a pool) may include at least 2, 5, 10, 25, 50, 100, 250, 500 or more types of affinity agents, each type of affinity agent differing from the other types with respect to the epitope(s) recognized. Alternatively or additionally, a plurality of affinity agents may include at most 500, 250, 100, 50, 25, 10, 5, or 2 types of affinity agents, each type of affinity agent differing from the other types with respect to the epitope(s) recognized. Different types of affinity agents in a pool can be uniquely labeled such that the different types can be distinguished from each other. In some configurations, at least two, and up to all, of the different types of affinity agents in a pool may be indistinguishably labeled with respect to each other. Alternatively or additionally to the use of unique labels, different types of affinity agents can be delivered and detected serially when evaluating one or more proteins (e.g. in an array).

A method of the present disclosure can be performed in a multiplex format. In multiplexed configurations, different analytes can be attached to different unique identifiers (e.g. proteins can be attached to unique analyte identification moieties). Multiplexed analytes can be manipulated and detected in parallel. For example, a fluid containing one or more different affinity agents can be delivered to a vessel containing proteins such that the proteins are in simultaneous contact with the affinity agent(s).

A protein or other analyte can be attached to a unique identifier (e.g. an analyte identification moiety) using any of a variety of means. The attachment can be covalent or non-covalent. Exemplary covalent attachments include chemical linkers such as those achieved using click chemistry or other linkages known in the art or described in U.S. Pat. Nos. 11,203,612 or 11,505,796 or US Pat. App. Pub. No 2023/0167488 A1, each of which is incorporated herein by reference. Non-covalent attachment can be mediated by receptor-ligand interactions (e.g. (strept) avidin-biotin, antibody-antigen, or complementary nucleic acid strands), for example, in which the receptor is attached to the unique identifier and the ligand is attached to the protein or vice versa.

One or more compositions set forth herein can be present in an apparatus or vessel. For example, a composition of the present disclosure can be present in a vessel, such as a flow cell. As a further option, the vessel can be engaged with a detection apparatus. The vessel can be permanently or temporarily engaged with the detection apparatus. A detection apparatus can be configured to detect contents of a vessel, for example, by acquiring signals arising from the vessel. For example, a detection apparatus can be configured to acquire optical signals through an optically transparent window of the vessel. Optionally, the detection apparatus can be configured for luminescence detection, for example, having an optical train that delivers radiation from an excitation source (e.g. a laser or lamp) through a window of the vessel. The detection apparatus can further include a camera or other detector that acquires signals transmitted through the window of the vessel and through an optical train. Optionally, excitation and emission can be transmitted through the same optical train; however, separate optical trains can also be useful.

A method of the present disclosure can include the step of coupling one or more analytes to a solid support or a surface thereof, for example, prior to performing a detection step set forth herein. The coupling of one or more analytes to a solid support surface may include covalent or non-covalent coupling of the one or more analytes to the solid support. Covalent coupling of an analyte to a solid support can include direct covalent coupling of an analyte to a solid support (e.g., formation of coordination bonds) or indirect covalent coupling between a reactive functional group of the analyte and a reactive functional group that is coupled to the solid support (e.g., a CLICK-type reaction). Non-covalent coupling can include the formation of any non-covalent interaction between an analyte and a solid support, including electrostatic or magnetic interactions, or non-covalent bonding interactions (e.g., ionic bonds, van der Waals interactions, hydrogen bonding, etc.). The skilled person will readily recognize that the particular analyte and the choice of solid support can affect the selection of a coupling chemistry for the compositions and methods set forth herein.

An analyte or affinity reagent can be attached to a retaining component such as a particle, array address, solid support or other substance. A particularly useful retaining component is a structured nucleic acid particle (SNAP). SNAPs can optionally include nucleic acid origami. A nucleic acid origami can include one or more nucleic acids folded into a variety of overall shapes such as a disk, tile, cylinder, cone, sphere, cuboid, tubule, pyramid, polyhedron, or combination thereof. Examples of structures formed with DNA origami are set forth in Zhao et al. Nano Lett. 11, 2997-3002 (2011); Rothemund Nature 440:297-302 (2006); Sigle et al, Nature Materials 20:1281-1289 (2021); or U.S. Pat. Nos. 8,501,923 or 9,340,416, each of which is incorporated herein by reference. In some configurations, a structured nucleic acid particle can include a nucleic acid nanoball and the nucleic acid nanoball can include a concatemeric repeat of amplified nucleotide sequences. The concatemeric amplicons can include complements of a circular template amplified by rolling circle amplification. Exemplary nucleic acid nanoballs and methods for their manufacture are described, for example, in U.S. Pat. No. 8,445,194, which is incorporated herein by reference. Further examples of structured nucleic acid particles are set forth in U.S. Pat. Nos. 11,203,612 or 11,505,796; or US Pat. App. Pub. No. 2022/0162684 A1 or 2023/0167488 A1, each of which is incorporated herein by reference. Other useful retaining components can include globular or branched polymers, such as dendrimers or dendrons.

The present disclosure provides compositions and methods for improving the binding of analytes to affinity reagents by increasing the avidity of the binding interaction. In particular embodiments, avidity between an analyte and an affinity reagent can be increased by association of a docker with the analyte and association of a tether with the affinity reagent. The docker and tether recognize each other and can thus bind to each other. Avidity of the interaction between the affinity reagent and analyte is a function not only of recognition between the paratope and epitope, but also recognition between the docker and tether. Additional aspects of docker/tether systems and other methods for forming binding interactions between analytes and binding reagents are described in U.S. Pat. No. 11,692,217 and U.S. Patent Publication Nos. 20240426839 and 20250066841, each of which is herein incorporated by reference in its entirety.

Compositions set forth herein can interact with each other via covalent bonds. Molecules, moieties thereof or atoms thereof can form covalent bonds with other molecules, moieties or atoms. Covalent interactions can be reversible or irreversible in the context of a method set forth herein. A covalent bond can arise due to a chemical reaction between a first reactive moiety and a second reactive moiety, optionally in the presence of a third intermediary or catalytic moiety. Covalent bonds can be formed via various chemical mechanisms, including addition, substitution, elimination, oxidation, and reduction. In some cases, a covalent binding interaction may be formed by a Click-type reaction, as set forth herein (e.g., methyltetrazine (mTz)-tetracyclooctylene (TCO), azide-dibenzocyclooctene (DBCO), thiol-epoxy). In some cases, a ligand-receptor-type binding interaction can form a covalent binding interaction. For example, SpyCatcher-SpyTag, SnoopCatcher-SnoopTag, and SdyCatcher-SdyTag are receptor-ligand binding pairs that can form covalent binding interactions due to isopeptide bond formation. Additional useful covalent binding interactions can include coordination bond formation, such as between a metal-containing substrate and a ligand. Exemplary coordination bonds can include silicon-silane, metal oxide-phosphate, and metal oxide-phosphonate. Useful reagents and mechanisms for forming covalent binding interactions, including bioorthogonal binding interactions, as set forth herein, are provided in U.S. Pat. Nos. 11,203,612 or 11,505,796, each of which is herein incorporated by reference in its entirety

Compositions set forth herein can interact with each other via non-covalent bonds. A non-covalent bond can include an electrostatic or magnetic interaction between a first moiety and a second moiety. A non-covalent bond can include electrostatic interactions such as ionic bonding, hydrogen bonding, halogen bonding, Van der Waals interactions, Pi-Pi stacking, Pi-ion interactions, Pi-polar interactions, or magnetic interactions. In some cases, a non-covalent bond may be formed by hybridization of a first oligonucleotide to a complementary second oligonucleotide. Such bonding is also known as Watson-Crick base-pairing. In some cases, a non-covalent interaction may be formed by a receptor-ligand binding pair, such as streptavidin-biotin. Other useful non-covalent interactions can include affinity reagent-target interactions, such as antibody-epitope or aptamer-epitope interactions.

Entities, such as affinity reagents and their binding targets, can be associated with each other and dissociated form each other in a method set forth herein. Association of a first entity to a second entity can involve a contacting step, in which the first entity is brought into proximity of the second entity, and an association step in which a first coupling moiety of the first entity forms a binding interaction with a second coupling moiety of the second entity. Dissociation of a first entity and a second entity need not be construed as a reversal of an association process between the first entity and the second entity. For example, a first entity comprising a first oligonucleotide coupled to a second entity comprising a second oligonucleotide by hybridization of the first oligonucleotide to the second oligonucleotide could be dissociated by dehybridization of the nucleic acids (thereby returning the first entity and the second entity as originally provided before association), or dissociated by enzymatic cleavage of the hybridized nucleic acids (thereby providing the first and the second entities with each individually further comprising an at least partially double-stranded cleavage product).

Systems or methods set forth herein may utilize one or more fluidic media to implement a process or step thereof. For array-based processes and systems, fluidic media may be provided for various process steps, including preparing arrays, attaching analytes to arrays, associating affinity agents to analytes, dissociating affinity agents from analytes, rinsing unbound moieties from array surfaces, performing detection processes on arrays, displacing a fluidic medium from contact with an array or other system components, and various other chemical and/or physical alterations of analytes or array components. A fluidic medium may be formulated to deliver a plurality of macromolecules (e.g., analytes, affinity agents) to an array as set forth herein. A fluidic medium may be formulated to mediate an interaction between macromolecules (e.g., an interaction between an analyte and an affinity agent).

A fluidic medium may be a single-phase or multi-phase fluidic medium. A multi-phase fluidic medium can include a gas phase and a liquid phase or at least two immiscible liquids. A multi-phase fluidic medium may comprise an interface between a first phase and a second phase. An interface between two fluidic phases may be laminar (e.g., an oil phase floating on an aqueous phase) or dispersed (e.g., bubbles, vesicles or droplets). A dispersed interface may be formed by a process such as emulsification. A divided interface may be stable (e.g., an emulsion) or unstable (e.g., a flocculating suspension). A multi-phase fluidic medium may comprise a colloidal agent that mediates an interface between a first phase and a second phase.

A fluidic medium can further contain solids, including particles (e.g., microparticles, nanoparticles). A fluidic medium comprising solids may be provided as a mixture, a suspension, or a slurry. It may be advantageous to provide a fluidic medium comprising a mixture or suspension of macromolecules. In some cases, solubility or suspendability of solids, such as particles or macromolecules, within a fluidic medium can be modulated by the composition of the fluidic medium. For example, alteration of fluidic properties such as solvent composition, ionic strength, and/or pH can induce precipitation, sedimentation, or flocculation of solvated or suspended solids.

The methods, compositions and apparatus of the present disclosure are particularly well suited for use with proteins. Although proteins are exemplified throughout the present disclosure, it will be understood that other analytes can be similarly used. Exemplary analytes include, but are not limited to, biomolecules, polysaccharides, nucleic acids, lipids, metabolites, hormones, vitamins, enzyme cofactors, therapeutic agents, candidate therapeutic agents or combinations thereof. An analyte can be a non-biological atom or molecule, such as a synthetic polymer, metal, metal oxide, ceramic, semiconductor, mineral, or a combination thereof.

One or more proteins that are used in a method, composition or apparatus herein, can be derived from a natural or synthetic source. Exemplary sources include, but are not limited to biological tissues, fluids, cells or subcellular compartments (e.g. organelles). For example, a sample can be derived from a tissue biopsy, biological fluid (e.g. blood, sweat, tears, plasma, extracellular fluid, urine, mucus, saliva, semen, vaginal fluid, synovial fluid, lymph, cerebrospinal fluid, peritoneal fluid, pleural fluid, amniotic fluid, intracellular fluid, extracellular fluid, etc.), fecal sample, hair sample, cultured cell, culture media, fixed tissue sample (e.g. fresh frozen or formalin-fixed paraffin-embedded) or product of a protein synthesis reaction. A protein source may include any sample where a protein is a native or expected constituent. For example, a primary source for a cancer biomarker protein may be a tumor biopsy sample or bodily fluid. Other sources include environmental samples or forensic samples.

Exemplary organisms from which proteins or other analytes can be derived include, for example, a mammal such as a rodent, mouse, rat, rabbit, guinea pig, ungulate, horse, sheep, pig, goat, cow, cat, dog, primate, non-human primate or human; a plant such as Arabidopsis thaliana, tobacco, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a dictyostelium discoideum; a fungi such as Pneumocystis carinii, Takifugu rubripes, yeast, Saccharamoyces cerevisiae or Schizosaccharomyces pombe; or a Plasmodium falciparum. Proteins can also be derived from a prokaryote such as a bacterium, Escherichia coli, staphylococci or Mycoplasma pneumoniae; an archae; a virus such as Hepatitis C virus, influenza virus, coronavirus, or human immunodeficiency virus; or a viroid. Proteins can be derived from a homogeneous culture or population of the above organisms or alternatively from a collection of several different organisms, for example, in a community or ecosystem.

In some cases, a protein or other biomolecule can be derived from an organism that is collected from a host organism. For example, a protein may be derived from a parasitic, pathogenic, symbiotic, or latent organism collected from a host organism. A protein can be derived from an organism, tissue, cell or biological fluid that is known or suspected of being linked with a disease state or disorder (e.g., cancer). Alternatively, a protein can be derived from an organism, tissue, cell or biological fluid that is known or suspected of not being linked to a particular disease state or disorder. For example, the proteins isolated from such a source can be used as a control for comparison to results acquired from a source that is known or suspected of being linked to the particular disease state or disorder. A sample may include a microbiome or substantial portion of a microbiome. In some cases, one or more proteins used in a method, composition or apparatus set forth herein may be obtained from a single source and no more than the single source. The single source can be, for example, a single organism (e.g. an individual human), single tissue, single cell, single organelle (e.g. endoplasmic reticulum, Golgi apparatus or nucleus), or single protein-containing particle (e.g., a viral particle or vesicle).

A method, composition or apparatus of the present disclosure can use or include a plurality of proteins having any of a variety of compositions such as a plurality of proteins composed of a proteome or fraction thereof. For example, a plurality of proteins can include solution-phase proteins, such as proteins in a biological sample or fraction thereof, or a plurality of proteins can include proteins that are immobilized, such as proteins attached to a particle or solid support. By way of further example, a plurality of proteins can include proteins that are detected, analyzed or identified in connection with a method, composition or apparatus of the present disclosure. The content of a plurality of proteins can be understood according to any of a variety of characteristics such as those set forth below or elsewhere herein.

A plurality of proteins can be characterized in terms of total protein mass. The total mass of protein in a liter of plasma has been estimated to be 70 g and the total mass of protein in a human cell has been estimated to be between 100 μg and 500 pg depending upon cells type. Sec Wisniewski et al. Molecular & Cellular Proteomics 13:10.1074/mcp.M113.037309, 3497-3506 (2014), which is incorporated herein by reference. A plurality of proteins used or included in a method, composition or apparatus set forth herein can include at least 1 μg, 10 pg, 100 μg, 1 ng, 10 ng, 100 ng, 1 mg, 10 mg, 100 mg, 1 mg, 10 mg, 100 mg or more protein by mass. Alternatively or additionally, a plurality of proteins may contain at most 100 mg, 10 mg, 1 mg, 100 mg, 10 mg, 1 mg, 100 ng, 10 ng, 1 ng, 100 μg, 10 pg, 1 μg or less protein by mass.

A plurality of proteins can be characterized in terms of percent mass relative to a given source such as a biological source (e.g. cell, tissue, or biological fluid such as blood). For example, a plurality of proteins may contain at least 60%, 75%, 90%, 95%, 99%, 99.9% or more of the total protein mass present in the source from which the plurality of proteins was derived. Alternatively or additionally, a plurality of proteins may contain at most 99.9%, 99%, 95%, 90%, 75%, 60% or less of the total protein mass present in the source from which the plurality of proteins was derived.

A plurality of proteins can be characterized in terms of total number of protein molecules. The total number of protein molecules in a Saccharomyces cerevisiae cell has been estimated to be about 42 million protein molecules. See Ho et al., Cell Systems (2018), DOI: 10.1016/j.cels.2017.12.004, which is incorporated herein by reference. A plurality of proteins used or included in a method, composition or apparatus set forth herein can include at least 1 protein molecule, 10 protein molecules, 100 protein molecules, 1×10⁴ protein molecules, 1×10⁶ protein molecules, 1×10⁸ protein molecules, 1×10¹⁰ protein molecules, 1 mole (6.02214076×10²³ molecules) of protein, 10 moles of protein molecules, 100 moles of protein molecules or more. Alternatively or additionally, a plurality of proteins may contain at most 100 moles of protein molecules, 10 moles of protein molecules, 1 mole of protein molecules, 1×10¹⁰ protein molecules, 1×10⁸ protein molecules, 1×10⁶ protein molecules, 1×10⁴ protein molecules, 100 protein molecules, 10 protein molecules, 1 protein molecule or less.

A plurality of proteins can be characterized in terms of the variety of full-length primary protein structures in the plurality. For example, the variety of full-length primary protein structures in a plurality of proteins can be equated with the number of different protein-encoding genes in the source for the plurality of proteins. Whether or not the proteins are derived from a known genome or from any genome at all, the variety of full-length primary protein structures can be counted independent of presence or absence of post translational modifications in the proteins. A human proteome is estimated to have about 20,000 different protein-encoding genes such that a plurality of proteins derived from a human can include up to about 20,000 different primary protein structures. See Aebersold et al., Nat. Chem. Biol. 14:206-214 (2018), which is incorporated herein by reference. Other genomes and proteomes in nature are known to be larger or smaller.

In relative terms, a plurality of proteins used or included in a method, composition or apparatus set forth herein may contain at least one representative for at least 60%, 75%, 90%, 95%, 99%, 99.9% or more of the proteins encoded by the genome of a source from which the sample was derived. Alternatively or additionally, a plurality of proteins may contain a representative for at most 99.9%, 99%, 95%, 90%, 75%, 60% or less of the proteins encoded by the genome of a source from which the sample was derived.

A plurality of proteins can be characterized in terms of the variety of primary protein structures in the plurality including transcribed splice variants. The human proteome has been estimated to include about 70,000 different primary protein structures when splice variants ac included. See Aebersold et al., Nat. Chem. Biol. 14:206-214 (2018), which is incorporated herein by reference. Moreover, the number of the partial-length primary protein structures can increase due to fragmentation that occurs in a sample. A plurality of proteins used or included in a method, composition or apparatus set forth herein can have a complexity of at least 2, 5, 10, 100, 1×10³, 1×10⁴, 7×10⁴, 1×10⁵, 1×10⁶ or more different primary protein structures. Alternatively or additionally, a plurality of proteins can have a complexity that is at most 1×10⁶, 1×10⁵, 7×10⁴, 1×10⁴, 1×10³, 100, 10, 5, 2 or fewer different primary protein structures.

A plurality of proteins can be characterized in terms of the variety of protein structures in the plurality including different primary structures and different proteoforms among the primary structures. Different molecular forms of proteins expressed from a given gene are considered to be different proteoforms. Proteoforms can differ, for example, due to differences in primary structure (e.g. shorter or longer amino acid sequences), different arrangement of domains (e.g. transcriptional splice variants), or different post translational modifications (e.g. presence or absence of phosphoryl, glycosyl, acetyl, or ubiquitin moieties). The human proteome is estimated to include hundreds of thousands of proteins when counting the different primary structures and proteoforms. See Aebersold et al., Nat. Chem. Biol. 14:206-214 (2018), which is incorporated herein by reference. A plurality of proteins used or included in a method, composition or apparatus set forth herein can have a complexity of at least 2, 5, 10, 100, 1×10³, 1×10⁴, 1×10⁵, 1×10⁶, 5×10⁶, 1×10⁷ or more different protein structures. Alternatively or additionally, a plurality of proteins can have a complexity that is at most 1×10⁷, 5×10⁶, 1×10⁶, 1×10⁵, 1×10⁴, 1×10³, 100, 10, 5, 2 or fewer different protein structures.

A plurality of proteins can be characterized in terms of the dynamic range for the different protein structures in the sample. The dynamic range can be a measure of the range of abundance for all different protein structures in a plurality of proteins, the range of abundance for all different primary protein structures in a plurality of proteins, the range of abundance for all different full-length primary protein structures in a plurality of proteins, the range of abundance for all different full-length gene products in a plurality of proteins, the range of abundance for all different proteoforms expressed from a given gene, or the range of abundance for any other set of different proteins set forth herein. The dynamic range for all proteins in human plasma is estimated to span more than 10 orders of magnitude from albumin, the most abundant protein, to the rarest proteins that have been measured clinically. See Anderson and Anderson Mol Cell Proteomics 1:845-67 (2002), which is incorporated herein by reference. The dynamic range for plurality of proteins set forth herein can be a factor of at least 10, 100, 1×10³, 1×10⁴, 1×10⁶, 1×10⁸, 1×10¹⁰, or more. Alternatively or additionally, the dynamic range for plurality of proteins set forth herein can be a factor of at most 1×10¹⁰, 1×10⁸, 1×10⁶, 1×10⁴, 1×10³, 100, 10 or less.

The present disclosure provides compositions, apparatus and methods that are useful for detecting, characterizing and identifying proteoforms. For example, the presence or absence of a particular post-translational modification or a particular post-translationally modified amino acid can be determined. In some embodiments, a proteoform can be characterized with respect to the location(s) of one or more post-translational modifications in the amino acid sequence of the proteoform. Locations can be identified, for example, at a specific position of the amino acid sequence for the proteoform. However, in some cases, the location of a post-translational modification in a proteoform can be determined relative to a particular structural motif of the proteoform. For example, a post-translational moiety of a proteoform can be located relative to a short sequence of amino acids in the proteoform or relative to another post-translational moiety in the proteoform.

Methods of the present disclosure are particularly well suited for manipulating and detecting proteoforms. The presence or absence of post-translational modifications (PTM) can be detected using a composition, apparatus or method set forth herein. A PTM can be detected using an affinity agent that recognizes the PTM or based on a chemical property of the PTM. In some configurations, methods set forth herein can be used to differentially manipulate proteoforms based on unique molecular properties or to distinguish one proteoform from another. Additional methods and aspects of characterizing proteoforms are provided in U.S. Pat. No. 12,092,642, and U.S. patent application Ser. Nos. 19/279,820 and 19/279,954, each of which is incorporated herein by reference in its entirety.

A post-translational modification may be one or more of myristoylation, palmitoylation, isoprenylation, prenylation, farnesylation, geranylgeranylation, lipoylation, flavin moiety attachment, Heme C attachment, phosphopantetheinylation, retinylidene Schiff base formation, dipthamide formation, ethanolamine phosphoglycerol attachment, hypusine, beta-Lysine addition, acylation, acetylation, deacetylation, formylation, alkylation, methylation, C-terminal amidation, arginylation, polyglutamylation, polyglyclyation, butyrylation, gamma-carboxylation, glycosylation, glycation, polysialylation, malonylation, hydroxylation, iodination, nucleotide addition, phosphoate ester formation, phosphoramidate formation, phosphorylation, adenylylation, uridylylation, propionylation, pyrolglutamate formation, S-glutathionylation, S-nitrosylation, S-sulfenylation, S-sulfinylation, S-sulfonylation, succinylation, sulfation, glycation, carbamylation, carbonylation, isopeptide bond formation, biotinylation, carbamylation, oxidation, reduction, pegylation, ISGylation, SUMOylation, ubiquitination, neddylation, pupylation, citrullination, deamidation, elminylation, disulfide bridge formation, isoaspartate formation, and racemization. Proteoforms can differ with regard to presence or absence of a post-translational modification, type of post-translational modification present, location of a post-translational modification, number of post-translational modifications present or combination thereof.

A post-translational modification may occur at a particular type of amino acid residue in a protein. For example, the phosphate moiety of a particular proteoform can be present on a serine, threonine, tyrosine, histidine, cysteine, lysine, aspartate or glutamate residue. In another example, an acetyl moiety of a particular proteoform can be present on the N-terminus or on a lysine of a protein. In another example, a serine or threonine residue of a proteoform can have an O-linked glycosyl moiety, or an asparagine residue of a proteoform can have an N-linked glycosyl moiety. In another example, a proline, lysine, asparagine, aspartate or histidine amino acid of a proteoform can be hydroxylated. In another example, a proteoform can be methylated at an arginine or lysine amino acid. In another example, a proteoform can be ubiquitinated at the N-terminal methionine or at a lysine amino acid.

A post-translationally modified version of a given amino acid can include a post-translational moiety at a side chain position that is unmodified in a standard version of the amino acid. Post-translationally modified lysines can include epsilon amines attached to post-translational moieties, whereas standard lysines have epsilon amines lacking the post-translational moieties. Post-translationally modified histidines can include side-chain tertiary amines attached to post-translational moieties, whereas in standard histidines the side-chain amines are secondary amines lacking the post-translational moieties. Post-translationally modified versions of aspartates or glutamates can include side-chain carbonyls, esters or amides attached to post-translational moieties, whereas in standard versions of aspartates or glutamates the side-chains have carboxyls lacking the post-translational moieties. Post-translationally modified versions of arginines can include side-chain amines attached to post-translational moieties, whereas in standard versions of arginines the side-chain amines lack the post-translational moieties. Post-translationally modified versions of cysteines can include thioethers attached to post-translational moieties, whereas standard versions of cysteines have sulfurs lacking the post-translational moieties. Post-translationally modified versions of serines, threonines or tyrosines can include ethers or esters attached to post-translational moieties, whereas standard versions of serines, threonines or tyrosines have hydroxyls lacking the post-translational moieties.

A method of the present disclosure can include a step of removing post-translational moieties from post-translationally modified amino acids, thereby forming standard amino acids. In some cases, an enzyme can be used to remove a post-translational moiety from an amino acid. An enzyme that removes a post-translational moiety independently of amino acid sequence context surrounding the post-translationally modified amino acid can be used. In other cases, a sequence-specific enzyme can be used to remove a post-translational moiety. A method of the present disclosure can include a step of adding post-translational moieties to unmodified amino acids, thereby forming proteoforms of proteins. Post-translational modifications of proteins may be performed in an in vitro or in vivo system.

A phosphatase enzyme can be used to remove a phosphate moiety from an amino acid. A broadscale (e.g. sequence agnostic)phosphatase such as alkaline phosphatase can be useful. Protein phosphatases are available for removing phosphate moieties from various types of amino acids. Exemplary protein phosphatases include, but are not limited to, tyrosine-specific kinases such as PTP1B; serine/threonine-specific phosphatases such as PP2C and PPP2CA; dual specificity phosphatases such as lambda protein phosphatase or VHR, both of which can remove phosphate moieties from serine, threonine or tyrosine residues; or histidine phosphatase such as PHP. Phosphatases or kinases that are specific to particular signal transduction pathways can be used to remove phosphates in a sequence specific manner if desired.

Several enzymes are available for removing post-translational moieties from lysines. Examples are set forth in Wang and Cole, Cell Chemical Biology 27:953-969 (2020) (which is incorporated herein by reference) and below. Lysine deacetylases can be used to remove acetyl moieties from lysines. For example, at least eighteen different protein lysine deacetylases (e.g. histone deacetylases) are known to remove acetyl moieties from lysines in human proteins. Lysine demethylases can be used to remove methyl moieties from lysines. Deubiquitinases (DUBs) are isopeptidases that sever the amide bond between a lysine side chain of a protein and the ubiquitin (Ub) C terminus. Many DUBs can cleave Ub-Ub amide linkages whereas others show selectivity for particular ubiquitinated proteins.

Optionally, glycan moieties can be released from proteins in a method of the present disclosure. For example, N-glycans or O-glycans can be released from glycoproteins using glycosidases. Any of a variety of enzymes can be used to remove glycans from proteins. For example, α-2-3,6,8,9-Neuraminidase can be used to cleave non-reducing terminal branched and unbranched sialic acids; β-1,4-galactosidase can be used to remove β-1,4-linked nonreducing terminal galactose from proteins; β-N-acetylgucosaminidase can be used to cleave non-reducing terminal β-linked N-acetylgucosamine from proteins; endo-a-N-acetylgalactosaminidase can be used to remove O-glycosylation, for example, removing serine- or threonine-linked unsubstituted Galb1,3GalNac; and PNGase F can be used to cleave oligosaccharides from asparagines. Exemplary reagents and methods for releasing glycans from proteins are set forth in Zhang et al. Frontiers in Chemistry, vol 8, Article 508 (2020) doi: 10.3389/fchem.2020.00508, which is incorporated herein by reference.

A plurality of extant proteins may contain two or more proteoforms of a single species of protein (e.g., at least 2, 3, 4, 5, 10, 20, 50, 100, or more than 100 proteoforms). Alternatively, a plurality of extant proteins may contain only a single proteoform of a single species. A plurality of extant proteins may contain at least one species of protein having two or more proteoforms (e.g., at least 2, 10, 50, 100, 500, 1000, 5000, 10000, or more than 10000 species of protein having two or more proteoforms). Alternatively, a plurality of extant proteins may contain at least one species of protein having only one proteoform (e.g., at least 2, 10, 50, 100, 500, 1000, 5000, 10000, or more than 10000 species of protein having only one proteoform).

A method of identifying extant proteins may further include identifying proteoforms of extant proteins. Accordingly, a method of identifying a proteoform of an individual protein can include the steps of: i) identifying a primary amino acid sequence of the protein based upon a binding profile of the protein, thereby identifying the protein, and ii) identifying a proteoform of the protein. Proteoform-specific affinity agents may be useful for identifying the proteoform of an extant protein. A proteoform-specific affinity agent can be a promiscuous affinity agent, for example binding to post-translational modifications (e.g., methylations, phosphorylations, glycosylations, etc.) of a plurality of protein species and/or proteoforms. A proteoform-specific affinity agent can be highly specific to a single proteoform of one or more protein species (e.g., only binding to a single post-translationally modified amino acid of a single protein species). A proteoform may be identified in part by detecting presence of binding of one or more affinity agents to an extant protein. Alternatively, a proteoform may be identified in part by an absence of detectable binding of one or more affinity agents to an extant protein (e.g., due to absence of a post-translational modification at an amino acid residue of the extant protein, due to absence of a bindable epitope due to splice variation of the extant protein, etc.).

In some cases, it may be preferable to contact extant proteins with a proteoform-specific affinity agent before contacting the extant proteins with other promiscuous or non-proteoform affinity agents. Presence of certain post-translational modification may inhibit binding of affinity agents to epitopes where said post-translational modification are present. Accordingly, a method may further comprise a step of removing post-translation modification (e.g., chemically or enzymatically) from extant proteins. After detecting binding of proteoform-specific affinity agents to extant proteins, and optionally removing one or more post-translational modification from the extant proteins, the extant proteins may be subsequently contacted with a series of promiscuous affinity agents, thereby providing binding profiles for each individual extant protein.

### EXAMPLES

### Example 1. Proteoform Characterization of Tau Protein

Proteins are individually obtained from a plurality of samples of human brain tissue. For each sample, the obtained protein sample is enriched to provide a sample that is predominantly Tau protein. Methods for enriching protein samples for a specific protein are described in U.S. patent application Ser. No. 19/279,954, which is herein incorporated by reference in its entirety. After enrichment, proteins of the enriched protein sample are attached to nucleic acid particles to form a composition containing single nucleic acid particles attached to single proteins. Methods for attaching proteins to single nucleic acid particles are described in U.S. Pat. No. 11,505,796 and U.S. Patent Publication No. 20240417720A1, each of which are herein incorporated by reference in their entireties. Each nucleic acid particle is attached to a unique analyte identification oligonucleotide barcode. Protein-nucleic acid particle conjugates are then bound to magnetic beads containing oligonucleotides that facilitate attachment of the nucleic acid particles to the bead surfaces. For each protein sample, beads containing attached Tau proteins are fluidically delivered into a well of a multi-well plate such that each filled contains Tau proteins from a different sample.

A magnetic field is applied to each well to pull magnetic beads to well surfaces. After magnetic separation, fluid is removed and discarded from each well. A fluid containing a plurality of affinity reagents is delivered to each well. Each affinity reagent is attached to an affinity reagent identification oligonucleotide containing a barcode that identifies the binding specificity of the attached affinity reagent. Seven different affinity reagents are utilized during the assay, so wells are divided into seven sets, each well of a set receiving the same type of affinity reagent as each other well in the set. Each affinity reagent recognizes and binds to a particular phosphorylated epitope of Tau protein. The seven affinity reagents are listed in Table II. The fluid containing each plurality of affinity reagents further comprises polymerase molecules and nucleotides.

TABLE II

| Affinity Reagent | Tau Binding Target |
|---|---|
| Anti-pT181 | Phosphorylated threonine residue 181 |
| Anti-pS202 | Phosphorylated serine residue 202 |
| Anti-pS205 | Phosphorylated serine residue 205 |
| Anti-pS214 | Phosphorylated serine residue 214 |
| Anti-pT217 | Phosphorylated threonine residue 217 |
| Anti-pT231 | Phosphorylated threonine residue 231 |
| Anti-pS396 | Phosphorylated serine residue 396 |

Affinity reagents are incubated in each well for 30 minutes. When bound to an analyte, a portion of the affinity reagent identification oligonucleotide of an affinity reagent is configured to form a hybridization interaction with a portion of the associated analyte identification moiety. Polymerase molecules extend the affinity reagent identification oligonucleotide in the presence of the nucleotides to include a reverse complementary copy of the barcode sequence of the analyte identification oligonucleotide. After the 30 minute incubation, each well is heated to dissociate affinity reagents from analytes, then a magnetic field is applied to the wells, thereby separating the magnetic beads from the fluid phase. The fluid volumes from each well are transferred to a well of a different set of wells such that each well of a set contains a same affinity reagent that was previously used for a different set of wells. After transferring pluralities of affinity reagents to differing wells, additional nucleotides are transferred into each well. The process is repeated until each set of analytes has been incubated four different times with each of the seven different affinity reagents.

After all incubation cycles are complete, the magnetic beads are pulled out of the fluid phase by a magnetic field. Each well is contacted with ultraviolet light to disrupt a photolabile bond that attaches the formed interaction identification moieties to each affinity reagent. For each well, separated interaction identification moieties are isolated then delivered to a sequencing device. The sequencing device determines for each sequenced interaction identification moiety an affinity reagent identification moiety and each analyte identification moiety that has been recorded with the affinity reagent identification moiety. The sequencing data are provided to a processor containing an algorithm that determines for each analyte which of the seven affinity reagents formed binding interactions with the analyte. The algorithm further categorizes each analyte according to the specific pattern of phosphorylation detected by binding interactions, then quantifies the number of detected proteins for each categorization.

### Example 2. One-Pot Proteoform Characterization of Tau Protein

Proteins are obtained and distributed into wells of a multi-well plate as per the method of Example 1. A magnetic field is applied to each well to pull magnetic beads to well surfaces. After magnetic separation, fluid is removed and discarded from each well. A fluid containing a plurality of affinity reagents is delivered to each well. The fluid delivered to each well contains each of the seven affinity reagents listed in Table II. The fluid further comprises polymerase molecules and nucleotides.

Each well is thermally cycled between binding states and polymerase extension states. Each well is incubated for 30 minutes at 16 degrees Celsius (C) to facilitate binding interactions between affinity reagents and analytes. After incubation, each well is heated to 30° C. to facilitate extension of information from analyte identification oligonucleotides onto affinity reagent identification oligonucleotides. Wells are held at 30° C. for 5 minutes. Thermal cycling between 16° C. and 30° C. is repeated until a total of 30 cycles has occurred.

After all incubation cycles are complete, the magnetic beads are pulled out of the fluid phase by a magnetic field. Each well is contacted with ultraviolet light to disrupt a photolabile bond that attaches the formed interaction identification moieties to each affinity reagent. For each well, separated interaction identification moieties are isolated then delivered to a sequencing device. The sequencing device determines for each sequenced interaction identification moiety an affinity reagent identification moiety and each analyte identification moiety that has been recorded with the affinity reagent identification moiety. The sequencing data are provided to a processor containing an algorithm that determines for each analyte which of the seven affinity reagents formed binding interactions with the analyte. The algorithm further categorizes each analyte according to the specific pattern of phosphorylation detected by binding interactions, then quantifies the number of detected proteins for each categorization.

### Example 3. Characterization of Unknown Proteins

The protein fraction of a human blood sample is obtained. Affinity chromatography is utilized to deplete albumins and immunoglobulins from the protein fraction. The depleted protein fraction is then attached to barcode-labeled nucleic acid particles as per the method of Example 1. The protein-nucleic acid particle conjugates are attached to magnetic beads. The protein fraction contains about 10¹⁰ total individual proteins. The magnetic beads are distributed equally amongst the wells of a 384 well plate such that about 2.6×10⁷ proteins are distributed into each well.

A magnetic field is applied to each well to pull magnetic beads to well surfaces. After magnetic separation, fluid is removed and discarded from each well. A fluid containing a plurality of affinity reagents is delivered to each well. Each plurality of affinity reagents contains 4 unique affinity reagents (i.e., four different binding specificities). Each well receives a substantially identical fluid (i.e., the fluid delivered to each well is substantially identical for each well). The affinity reagents in the fluid delivered to each well have a binding specificity for four different amino acid sequences of 3 to 5 amino acids in length. The fluid further comprises polymerase molecules and nucleotides.

Affinity reagents are incubated in each well for 30 minutes. When bound to an analyte, a portion of the affinity reagent identification oligonucleotide of an affinity reagent is configured to form a hybridization interaction with a portion of the associated analyte identification moiety. Polymerase molecules extend the affinity reagent identification oligonucleotide in the presence of the nucleotides to include a reverse complementary copy of the barcode sequence of the analyte identification oligonucleotide. After the 30 minute incubation, each well is heated to dissociate affinity reagents from analytes, then a magnetic field is applied to the wells, thereby separating the magnetic beads from the fluid phase.

Sequentially, the fluid containing affinity reagents is withdrawn from each well. After the fluid is withdrawn, the fluid is passed through a field of ultraviolet light that cleaves the interaction identification moiety from each affinity reagent. For each well, the interaction identification moieties are collected and delivered to a sequencing device. The sequencing device determines for each sequenced interaction identification moiety an affinity reagent identification moiety and any analyte identification moieties that have been recorded with the affinity reagent identification moiety. The sequencing device also includes information on the well from which the interaction identification moieties were delivered. The sequencing data are provided to a processor containing an algorithm that determines for each analyte which analytes were bound by the affinity reagents. A binding profile is formed for each analyte, in which the binding profile includes for each analyte a presence or absence of a detected analyte identification oligonucleotide sequence for each affinity reagent tested.

Additional cycles of analyte characterization are performed. A fluid containing affinity reagents is delivered to each well during each cycle. In total, 300 unique affinity reagents are tested in sets of four (e.g., at least 75 cycles of incubation and sequencing). The binding profile containing the presence or absence of binding of each of the 300 affinity reagents to each analyte is provided to a processor-based algorithm that is configured to provide a protein characterization (e.g., protein identity, proteoform, protein type, etc.) for each analyte based upon the analyte's binding profile.

### Example 4. Bulk Characterization of Tau Proteoforms

Unknown proteins obtained from a sample of human cerebrospinal fluid are characterized according to the method of Example 3. After contacting the proteins with the 300 unique affinity reagents, the proteins are further characterized by contact with the seven phospho-Tau specific affinity reagents listed in Table II. Each affinity reagent is attached to an oligonucleotide containing a sequence that is complementary to a portion of each analyte identification moiety and further containing a priming sequence. Affinity reagents are delivered in a fluid containing polymerase molecules and nucleotides. After incubation between analytes and affinity reagents, beads attached to analytes are magnetically separated from the fluid phase. A second fluid containing fluorescently-labeled nucleotides and priming oligonucleotides is delivered to each well, in which the priming oligonucleotides are configured to form a polymerase chain reaction (PCR) between the priming sequence of the affinity reagent oligonucleotides and the complementary sequence of the analyte identification moieties. Each well is thermocycled to perform a PCR reaction. A quantity of protein molecules forming binding interactions with affinity reagents in each well is measured by quantitative PCR, as measured by the release of fluorescent dye molecules from each incorporated fluorescently-labeled nucleotide. The method is repeated until each of the seven affinity reagents have been tested. The qPCR results provide a measure of the amount of phosphorylated Tau protein molecules in the cerebrospinal fluid sample.

1. A method of forming a binding profile, comprising:
(a) binding affinity reagents to analytes of a plurality of analytes, wherein each of the affinity reagents is individually joined with an affinity reagent identifier moiety, and wherein each of the analytes is individually joined with a unique analyte identifier moiety;
(b) for each analyte bound to an affinity reagent, coupling together the affinity reagent identifier moiety and the unique analyte identifier moiety, thereby forming an interaction identification moiety comprising an analyte-specific code from the unique analyte identifier moiety;
(c) repeating steps (a) and (b) at least once with differing affinity reagents;
(d) detecting for each interaction identification moiety from steps (b) and (c) the analyte-specific code; and
(e) for each analyte of the plurality of analytes, forming a binding profile for the analyte, wherein the binding profile comprises a presence or absence of a detected analyte-specific code for the analyte for each of the at least two differing affinity reagents.

2.-45. (canceled)

46. The method of claim 1, wherein the plurality of analytes comprises at least 106 analytes.

47. The method of claim 1, wherein the plurality of analytes comprises two or more species of analytes.

48. The method of claim 47, further comprising, based upon binding profiles of the analytes of the plurality of analytes, identifying the presence of the first species of analyte and the second species of analyte in the plurality of analytes.

49. The method of claim 1, wherein the plurality of analytes comprises a plurality of proteins.

50. The method of claim 49, wherein the plurality of proteins comprises two or more species of proteins, as determined by full-length primary amino acid structure.

51. The method of claim 50, wherein the plurality of proteins comprises two or more species of proteins, as determined by proteoform or isoform.

52. The method of claim 1, wherein forming the interaction identification moiety comprises ligating a first oligonucleotide comprising the analyte-specific code to a second oligonucleotide comprising the affinity reagent-specific code.

53. The method of claim 1, wherein forming the interaction identification moiety comprises hybridizing a first oligonucleotide comprising the analyte-specific code to a second oligonucleotide comprising the affinity reagent-specific code.

54. The method of claim 53, further comprising extending the first oligonucleotide or the second oligonucleotide enzymatically.

55. The method of claim 1, wherein the analyte identifier moiety comprises a polymer strand and wherein the affinity reagent identifier comprises an enzyme.

56. The method of claim 55, wherein forming the interaction identification moiety comprises altering the polymer strand with the enzyme.

57. The method of claim 1, wherein step (c) comprises: (i) separating the plurality of analytes from a first fluid phase; (ii) removing the first fluid phase, wherein the first fluid phase comprises the affinity reagents; and (iii) contacting a second fluid phase to the plurality of analytes, wherein the second fluid phase comprises a second set of affinity reagents.

58. The method of claim 57, wherein a binding specificity of the affinity reagents differs from a binding specificity of affinity reagents of the second set of affinity reagents.

59. The method of claim 1, further comprising characterizing each analyte of the plurality of analytes based upon the binding profile of the analyte.

60. The method of claim 59, wherein characterizing each analyte of the plurality of analytes comprises identifying an analyte of the plurality of analytes.

61. The method of claim 59, wherein characterizing each analyte of the plurality of analytes comprises identifying a proteoform of an analyte of the plurality of analytes.

62. The method of claim 61, wherein identifying the proteoform of the analyte of the plurality of analytes comprises identifying two or more proteoforms of a species of analyte of the plurality of analytes.

63. A composition, comprising:
(a) a solid support, wherein the solid support is attached to a plurality of particles, wherein, for each particle of the plurality of particles, the particle is attached to a unique analyte identifier moiety and a single unknown analyte;
(b) a plurality of affinity reagents; and
(c) a plurality of interaction identification moieties, wherein each interaction identification moiety comprises an analyte-specific code of an analyte identifier moiety.

64. A system, comprising:
(a) a plurality of vessels, wherein each vessel comprises a plurality of particles, wherein the plurality of particles is attached to a plurality of analytes, wherein each analyte of the plurality of analytes is co-localized with an analyte identifier moiety on a particle of the plurality of particles, wherein each analyte identifier moiety comprises a unique analyte-specific code;
(b) a library of affinity reagents, wherein the library of affinity reagents comprises two or more pluralities of affinity reagents, wherein each plurality of affinity reagents of the two or more pluralities of affinity reagents differs with respect to binding specificity from any other plurality of affinity reagents of the two or more pluralities of affinity reagents, and wherein each affinity reagent of the library of affinity reagents is attached to an affinity reagent identifier moiety;
(c) a detection device, wherein the detection device is configured to receive a plurality of interaction identification moieties, and wherein the detection device is further configured to detect for each interaction identification moiety of the plurality of interaction identification moieties an analyte-specific code; and
(d) a fluid transfer device, wherein the fluid transfer device is configured to deliver a plurality of affinity reagents from the library of affinity reagents to a vessel of the plurality of vessels, and wherein the fluid transfer device is further configured to deliver the plurality of interaction identification moieties from a vessel of the plurality of vessels to the detection device.
